SEQUENCE LISTING (164 sequences)

SEQ ID NO: 1 - DNA, 780 bp, Klebsiella pneumoniae
atgaatcattctgctgaatgcacctgcgaagagagtctatgcgaaaccctgcgggcgttttccgcgcagcatcccgagagcgtgctctatcagacatcgctcatgagcgccctgctgagcggggtttacgaaggcagcaccaccatcgcggacctgctgaaacacggcgatttcggcctcggcacctttaatgagctggacggggagctgatcgccttcagcagtcaggtctatcagctgcgcgccgacggcagcgcgcgcaaagcccagccggagcagaaaacgccgttcgcggtgatgacctggttccagccgcagtaccggaaaacctttgaccatccggtgagccgccagcagctgcacgaggtgatcgaccagcaaatcccctctgacaacctgttctgcgccctgcgcatcgacggccatttccgccatgcccatacccgcaccgtgccgcgccagacgccgccgtaccgggcgatgaccgacgtcctcgacgatcagccggtgttccgctttaaccagcgcgaaggggtgctggtcggcttccggaccccgcagcatatgcaggggatcaacgtcgccgggtatcacgagcactttattaccgatgaccgcaaaggcggcggtcacctgctggattaccagctcgaccatggggtgctgaccttcggcgaaattcacaagctgatgatcgacctgcccgccgacagcgcgttcctgcaggctaatctgcatcccgataatctcgatgccgccatccgttccgtagaaagttaa

SEQ ID NO: 2 - protein, 259 aa, Klebsiella pneumoniae
MNHSAECTCEESLCETLRAFSAQHPESVLYQTSLMSALLSGVYEGSTTIADLLKHGDFGLGTFNELDGELIAFSSQVYQLRADGSARKAQPEQKTPFAVMTWFQPQYRKTFDHPVSRQQLHEVIDQQIPSDNLFCALRIDGHFRHAHTRTVPRQTPPYRAMTDVLDDQPVFRFNQREGVLVGFRTPQHMQGINVAGYHEHFITDDRKGGGHLLDYQLDHGVLTFGEIHKLMIDLPADSAFLQANLHPDNLDAAIRSVES

SEQ ID NO: 3 - DNA, 1680 bp, Klebsiella pneumoniae
atggacaaacagtatccggtacgccagtgggcgcacggcgccgatctcgtcgtcagtcagctggaagctcagggagtacgccaggtgttcggcatccccggcgccaaaattgacaaggtcttcgactcactgctggattcctcgattcgcattattccggtacgccacgaagccaacgccgcgtttatggccgccgccgtcggacgcattaccggcaaagcgggcgtggcgctggtcacctccggtccgggctgttccaacctgatcaccggcatggccaccgcgaacagcgaaggcgacccggtggtggccctgggcggcgcggtaaaacgcgccgataaagcgaagcaggtccaccagagtatggatacggtggcgatgttcagcccggtcaccaaatacgccgtcgaggtgacggcgccggatgcgctggcggaagtggtctccaacgccttccgcgccgccgagcagggccggccgggcagcgcgttcgttagcctgccgcaggatgtggtcgatggcccggtcagcggcaaagtgctgccggccagcggggccccgcagatgggcgccgcgccggatgatgccatcgaccaggtggcgaagcttatcgcccaggcgaagaacccgatcttcctgctcggcctgatggccagccagccggaaaacagcaaggcgctgcgccgtttgctggagaccagccatattccagtcaccagcacctatcaggccgccggagcggtgaatcaggataacttctctcgcttcgccggccgggttgggctgtttaacaaccaggccggggaccgtctgctgcagctcgccgacctggtgatctgcatcggctacagcccggtggaatacgaaccggcgatgtggaacagcggcaacgcgacgctggtgcacatcgacgtgctgcccgcctatgaagagcgcaactacaccccggatgtcgagctggtgggcgatatcgccggcactctcaacaagctggcgcaaaatatcgatcatcggctggtgctctccccgcaggcggcggagatcctccgcgaccgccagcaccagcgcgagctgctggaccgccgcggcgcgcagctgaaccagtttgccctgcatccgctgcgcatcgttcgcgccatgcaggacatcgtcaacagcgacgtcacgttgaccgtggacatgggcagcttccatatctggattgcccgctacctgtacagcttccgcgcccgtcaggtgatgatctccaacggccagcagaccatgggcgtcgccctgccctgggctatcggcgcctggctggtcaatcctgagcgaaaagtggtctccgtctccggcgacggcggcttcctgcagtcgagcatggagctggagaccgccgtccgcctgaaagccaacgtactgcacctgatctgggtcgataacggctacaacatggtggccattcaggaagagaaaaaataccagcgcctgtccggcgtcgagttcgggccgatggattttaaagcctatgccgaatccttcggcgcgaaagggtttgccgtggaaagcgccgaggcgctggagccgaccctgcacgcggcgatggacgtcgacggcccggcggtggtggccattccggtggattatcgcgataacccgctgctgatgggccagctgcatctgagtcagattctgtaa

SEQ ID NO: 4 - protein, 559 aa, Klebsiella pneumoniae
MDKQYPVRQWAHGADLVVSQLEAQGVRQVFGIPGAKIDKVFDSLLDSSIRIIPVRHEANAAFMAAAVGRITGKAGVALVTSGPGCSNLITGMATANSEGDPVVALGGAVKRADKAKQVHQSMDTVAMFSPVTKYAVEVTAPDALAEVVSNAFRAAEQGRPGSAFVSLPQDVVDGPVSGKVLPASGAPQMGAAPDDAIDQVAKLIAQAKNPIFLLGLMASQPENSKALRRLLETSHIPVTSTYQAAGAVNQDNFSRFAGRVGLFNNQAGDRLLQLADLVICIGYSPVEYEPAMWNSGNATLVHIDVLPAYEERNYTPDVELVGDIAGTLNKLAQNIDHRLVLSPQAAEILRDRQHQRELLDRRGAQLNQFALHPLRIVRAMQDIVNSDVTLTVDMGSFHIWIARYLYSFRARQVMISNGQQTMGVALPWAIGAWLVNPERKVVSVSGDGGFLQSSMELETAVRLKANVLHLIWVDNGYNMVAIQEEKKYQRLSGVEFGPMDFKAYAESFGAKGFAVESAEALEPTLHAAMDVDGPAVVAIPVDYRDNPLLMGQLHLSQIL

SEQ ID NO: 5 - DNA, 771 bp, Klebsiella pneumoniae
atgaaaaaagtcgcacttgttaccggcgccggccaggggattggtaaagctatcgcccttcgtctggtgaaggatggatttgccgtggccattgccgattataacgacgccaccgccaaagcggtcgcctcggaaatcaaccaggccggcggacacgccgtggcggtgaaagtggatgtctccgaccgcgatcaggtatttgccgccgttgaacaggcgcgcaaaacgctgggcggcttcgacgtcatcgtcaataacgccggtgtggcaccgtctacgccgatcgagtccattaccccggagattgtcgacaaagtctacaacatcaacgtcaaaggggtgatctggggtattcaggcggcggtcgaggcctttaagaaagaggggcacggcgggaaaatcatcaacgcctgttcccaggccggccacgtcggcaacccggagctggcggtgtatagctccagtaaattcgcggtacgcggcttaacccagaccgccgctcgcgacctcgcgccgctgggcatcacggtcaacggctactgcccggggattgtcaaaacgccaatgtgggccgaaattgaccgccaggtgtccgaagccgccggtaaaccgctgggctacggtaccgccgagttcgccaaacgcatcactctcggtcgtctgtccgagccggaagatgtcgccgcctgcgtctcctatcttgccagcccggattctgattacatgaccggtcagtcgttgctgatcgacggcgggatggtatttaactaa

SEQ ID NO: 6 - protein, 256 aa, Klebsiella pneumoniae
MKKVALVTGAGQGIGKAIALRLVKDGFAVAIADYNDATAKAVASEINQAGGHAVAVKVDVSDRDQVFAAVEQARKTLGGFDVIVNNAGVAPSTPIESITPEIVDKVYNINVKGVIWGIQAAVEAFKKEGHGGKIINACSQAGHVGNPELAVYSSSKFAVRGLTQTAARDLAPLGITVNGYCPGIVKTPMWAEIDRQVSEAAGKPLGYGTAEFAKRITLGRLSEPEDVAACVSYLASPDSDYMTGQSLLIDGGMVFN

SEQ ID NO: 7 - DNA, 1665 bp, Klebsiella oxytoca
atgagatcgaaaagatttgaagcactggcgaaacgccctgtgaatcaggacggcttcgttaaggagtggatcgaagaaggctttatcgcgatggaaagcccgaacgacccaaaaccgtcgattaaaatcgttaacggcgcggtgaccgagctggacgggaaaccggtaagcgattttgacctgatcgaccactttatcgcccgctacggtatcaacctgaaccgcgccgaagaagtgatggcgatggattcggtcaagctggccaacatgctgtgcgatccgaacgttaaacgcagcgaaatcgtcccgctgaccaccgcgatgacgccggcgaaaattgtcgaagtggtttcgcatatgaacgtcgtcgagatgatgatggcgatgcagaaaatgcgcgcccgccgcaccccgtcccagcaggcgcacgtcaccaacgtcaaagataacccggtacagattgccgccgacgccgccgaaggggcatggcgcggatttgacgaacaggaaaccaccgttgcggtagcgcgctatgcgccgttcaacgccatcgcgctgctggtgggctcgcaggtaggccgtccgggcgtgctgacgcagtgctcgctggaagaagccaccgagctgaagctcggcatgctgggccacacctgctacgccgaaaccatctccgtctacggcaccgagccggtctttaccgacggcgacgacacgccgtggtcgaagggcttcctcgcctcgtcctacgcctctcgcgggctgaaaatgcgctttacctccggctccggctcggaagtgcagatgggctacgccgaaggcaaatccatgctttatctggaagcgcgctgcatctacatcaccaaagccgcgggcgtacagggtctgcaaaacggttccgtaagctgcatcggcgtgccgtctgcggtgccttccggcattcgcgcggtgctggcggaaaacctgatctgttcgtcgctggatctggagtgcgcctccagcaacgaccagaccttcacccactccgatatgcgtcgtaccgcgcgcctgctgatgcagttcctgccgggcaccgactttatctcctccggttattccgcggtgccgaactacgacaacatgttcgccggctccaacgaagatgccgaagactttgacgactacaacgtcatccagcgcgacctgaaggtggacggcggtttgcgtccggttcgcgaagaggacgtcatcgccatccgtaacaaagccgcccgcgcgctgcaggccgtgtttgccggaatggggctgccgccgattaccgatgaagaagttgaagccgcgacctacgcccacggttcgaaagatatgccggagcgcaacatcgtcgaagacatcaagttcgcccaggaaatcatcaataaaaaccgcaacggtctggaagtggtgaaagcgctggcgcagggcggattcaccgacgtggcccaggacatgctcaacatccagaaagctaagctgaccggggactacctgcatacctccgcgattatcgtcggcgacgggcaggtgctgtcagccgtcaacgacgtcaacgactatgccggtccggcaacgggctatcgcctgcagggcgaacgctgggaagagattaaaaacatccctggcgctcttgatcccaacgagattgattaa

SEQ ID NO: 8 - protein, 554 aa, Klebsiella oxytoca
MRSKRFEALAKRPVNQDGFVKEWIEEGFIAMESPNDPKPSIKIVNGAVTELDGKPVSDFDLIDHFIARYGINLNRAEEVMAMDSVKLANMLCDPNVKRSEIVPLTTAMTPAKIVEVVSHMNVVEMMMAMQKMRARRTPSQQAHVTNVKDNPVQIAADAAEGAWRGFDEQETTVAVARYAPFNAIALLVGSQVGRPGVLTQCSLEEATELKLGMLGHTCYAETISVYGTEPVFTDGDDTPWSKGFLASSYASRGLKMRFTSGSGSEVQMGYAEGKSMLYLEARCIYITKAAGVQGLQNGSVSCIGVPSAVPSGIRAVLAENLICSSLDLECASSNDQTFTHSDMRRTARLLMQFLPGTDFISSGYSAVPNYDNMFAGSNEDAEDFDDYNVIQRDLKVDGGLRPVREEDVIAIRNKAARALQAVFAGMGLPPITDEEVEAATYAHGSKDMPERNIVEDIKFAQEIINKNRNGLEVVKALAQGGFTDVAQDMLNIQKAKLTGDYLHTSAIIVGDGQVLSAVNDVNDYAGPATGYRLQGERWEEIKNIPGALDPNEID

SEQ ID NO: 9 - DNA, 675 bp, Klebsiella oxytoca
atggaaattaatgaaaaattgctgcgccagataattgaagacgtgctcagcgagatgaagggcagcgataaaccggtctcgtttaatgcgccggcggcctccgcggcgccccaggccacgccgcccgccggcgacggcttcctgacggaagtgggcgaagcgcgtcagggaacccagcaggacgaagtgattatcgccgtcggcccggctttcggcctggcgcagaccgtcaatatcgtcggcatcccgcataagagcattttgcgcgaagtcattgccggtattgaagaagaaggcattaaggcgcgcgtgattcgctgctttaaatcctccgacgtggccttcgtcgccgttgaaggtaatcgcctgagcggctccggcatctctatcggcatccagtcgaaaggcaccacggtgatccaccagcaggggctgccgccgctctctaacctggagctgttcccgcaggcgccgctgctgaccctggaaacctatcgccagatcggcaaaaacgccgcccgctatgcgaaacgcgaatcgccgcagccggtcccgacgctgaatgaccagatggcgcggccgaagtaccaggcgaaatcggccattttgcacattaaagagaccaagtacgtggtgacgggcaaaaacccgcaggaactgcgcgtggcgctttga

SEQ ID NO: 10 - protein, 224 aa, Klebsiella oxytoca
MEINEKLLRQIIEDVLSEMKGSDKPVSFNAPAASAAPQATPPAGDGFLTEVGEARQGTQQDEVIIAVGPAFGLAQTVNIVGIPHKSILREVIAGIEEEGIKARVIRCFKSSDVAFVAVEGNRLSGSGISIGIQSKGTTVIHQQGLPPLSNLELFPQAPLLTLETYRQIGKNAARYAKRESPQPVPTLNDQMARPKYQAKSAILHIKETKYVVTGKNPQELRVAL

SEQ ID NO: 11 - DNA, 522 bp, Klebsiella oxytoca
atgaataccgacgcaattgaatcgatggtacgcgacgtattgagccgcatgaacagcctgcagggcgaggcgcctgcggcggctccggcggctggcggcgcgtcccgtagcgccagggtcagcgactacccgctggcgaacaagcacccggaatgggtgaaaaccgccaccaataaaacgctggacgactttacgctggaaaacgtgctgagcaataaagtcaccgcccaggatatgcgtattaccccggaaaccctgcgcttacaggcttctattgccaaagacgcgggccgcgaccggctggcgatgaacttcgagcgcgccgccgagctgaccgcggtaccggacgatcgcattcttgaaatctacaacgccctccgcccctatcgctcgacgaaagaggagctgctggcgatcgccgacgatctcgaaagccgctatcaggcgaagatttgcgccgctttcgttcgcgaagcggccacgctgtacgtcgagcgtaaaaaactcaaaggcgacgattaa

SEQ ID NO: 12 - protein, 173 aa, Klebsiella oxytoca
MNTDAIESMVRDVLSRMNSLQGEAPAAAPAAGGASRSARVSDYPLANKHPEWVKTATNKTLDDFTLENVLSNKVTAQDMRITPETLRLQASIAKDAGRDRLAMNFERAAELTAVPDDRILEIYNALRPYRSTKEELLAIADDLESRYQAKICAAFVREAATLYVERKKLKGDD

SEQ ID NO: 13 - DNA, 1041 bp, Rhodococcus ruber
atgaaagccctccagtacaccgagatcggctccgagccggtcgtcgtcgacgtccccaccccggcgcccgggccgggtgagatcctgctgaaggtcaccgcggccggcttgtgccactcggacatcttcgtgatggacatgccggcagagcagtacatctacggtcttcccctcaccctcggccacgagggcgtcggcaccgtcgccgaactcggcgccggcgtcaccggattcgagacgggggacgccgtcgccgtgtacgggccgtgggggtgcggtgcgtgccacgcgtgcgcgcgcggccgggagaactactgcacccgcgccgccgagctgggcatcaccccgcccggtctcggctcgcccgggtcgatggccgagtacatgatcgtcgactcggcgcgccacctcgtcccgatcggggacctcgaccccgtcgcggcggttccgctcaccgacgcgggcctgacgccgtaccacgcgatctcgcgggtcctgcccctgctgggacccggctcgaccgcggtcgtcatcggggtcggcggactcgggcacgtcggcatccagatcctgcgcgccgtcagcgcggcccgcgtgatcgccgtcgatctcgacgacgaccgactcgcgctcgcccgcgaggtcggcgccgacgcggcggtgaagtcgggcgccggggcggcggacgcgatccgggagctgaccggcggtgagggcgcgacggcggtgttcgacttcgtcggcgcccagtcgacgatcgacacggcgcagcaggtggtcgcgatcgacgggcacatctcggtggtcggcatccatgccggcgcccacgccaaggtcggcttcttcatgatcccgttcggcgcgtccgtcgtgacgccgtactggggcacgcggtccgagctgatggacgtcgtggacctggcccgtgccggccggctcgacatccacaccgagacgttcaccctcgacgagggacccacggcctaccggcggctacgcgagggcagcatccgcggccgcggggtggtcgtcccgggctga

SEQ ID NO: 14 - protein, 346 aa, Klebsiella oxytoca
MKALQYTEIGSEPVVVDVPTPAPGPGEILLKVTAAGLCHSDIFVMDMPAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDLDDDRLALAREVGADAAVKSGAGAADAIRELTGGEGATAVFDFVGAQSTIDTAQQVVAIDGHISVVGIHAGAHAKVGFFMIPFGASVVTPYWGTRSELMDVVDLARAGRLDIHTETFTLDEGPTAYRRLREGSIRGRGVVVPG

SEQ ID NO: 15 - DNA, 29 bp, Artificial Sequence
  Primer
caccatggacaaacagtatccggtacgcc

SEQ ID NO: 16 - DNA, 25 bp, Artificial Sequence
  Primer
cgaagggcgatagctttaccaatcc

SEQ ID NO: 17 - DNA, 32 bp, Artificial Sequence
  Primer
caccatgaatcattctgctgaatgcacctgcg

SEQ ID NO: 18 - DNA, 22 bp, Artificial Sequence
  Primer
gatactgtttgtccatgtgacc

SEQ ID NO: 19 - DNA, 28 bp, Artificial Sequence
  Primer
caccatgaaaaaagtcgcacttgttacc

SEQ ID NO: 20 - DNA, 15 bp, Artificial Sequence
  Primer
ttagttaaataccat

SEQ ID NO: 21 - DNA, 23 bp, Artificial Sequence
  Primer
caccatgagatcgaaaagatttg

SEQ ID NO: 22 - DNA, 22 bp, Artificial Sequence
  Primer
cttagagaagttaatcgtcgcc

SEQ ID NO: 23 - DNA, 25 bp, Artificial Sequence
  Primer
caccatgaaagccctccagtacacc

SEQ ID NO: 24 - DNA, 18 bp, Artificial Sequence
  Primer
cgtcgtgtcatgcccggg

SEQ ID NO: 25 - DNA, 36 bp, Artificial Sequence
  Primer
gatcgaattcgtttaaacttagttttctaccgcacg

SEQ ID NO: 26 - DNA, 34 bp, Artificial Sequence
  Primer
gatcgcatgcaagctttcatatagtcggaattcc

SEQ ID NO: 27 - DNA, 43 bp, Artificial Sequence
  Primer
gatcgaattcgtttaaacaaaggaggtctgattcatgagatcg

SEQ ID NO: 28 - DNA, 22 bp, Artificial Sequence
  Primer
gatcggattcttaatcgtcgcc

SEQ ID NO: 29 - DNA, 36 bp, Artificial Sequence
  Primer
gatcggatccaaaggaggtcgggcgcatgaaagccc

SEQ ID NO: 30 - DNA, 32 bp, Artificial Sequence
  Primer
gatctctagaaagctttcagcccgggacgacc

SEQ ID NO: 31 - DNA, 21 bp, Artificial Sequence
  Primer
actttctttcgcctgtttcac

SEQ ID NO: 32 - DNA, 79 bp, Artificial Sequence
  Primer
catgaagcttgtttaaactcggtgaccttgaaaataatgaaaacttatattgttttgaaaataatgaaaacttatattg

SEQ ID NO: 33 - DNA, 39 bp, Artificial Sequence
  Primer BABC F
gagctcgaattcaaaggaggaagtgtatatgaatcattc

SEQ ID NO: 34 - DNA, 35 bp, Artificial Sequence
  Primer BAB R
ggatcctctagaattagttaaataccatcccgccg

SEQ ID NO: 35 - DNA, 17 bp, Artificial Sequence
  Primer M13 Forward
gtaaaacgacggccagt

SEQ ID NO: 36 - DNA, 16 bp, Artificial Sequence
  Primer M13 Reverse
aacagctatgaccatg

SEQ ID NO: 37 - DNA, 20 bp, Artificial Sequence
  Primer N83 SeqF2
gctggattaccagctcgacc

SEQ ID NO: 38 - DNA, 20 bp, Artificial Sequence
  Primer N83SeqF3
cggacgcattaccggcaaag

SEQ ID NO: 39 - DNA, 20 bp, Artificial Sequence
  Primer N84 SeqR4
cgaagcgagagaagttatcc

SEQ ID NO: 40 - DNA, 38 bp, Artificial Sequence
  Primer BC Spe F
actagtaaaggaggaaagagtatgaagaaggtcgcact

SEQ ID NO: 41 - DNA, 26 bp, Artificial Sequence
  Primer BC Xba R
tctagaaagcaggggcaagccatgtc

SEQ ID NO: 42 - DNA, 20 bp, Artificial Sequence
  Primer Trc F
ttgacaattaatcatccggc

SEQ ID NO: 43 - DNA, 20 bp, Artificial Sequence
  Primer Trc R
cttctctcatccgccaaaac

SEQ ID NO: 44 - DNA, 38 bp, Artificial Sequence
  Primer DDo For
aagcttaaaggaggctgattcatgagatcgaaaagatt

SEQ ID NO: 45 - DNA, 27 bp, Artificial Sequence
  Primer DDo Rev
tctagattattcatcctgctgttctcc

SEQ ID NO: 46 - DNA, 22 bp, Artificial Sequence
  Primer DDko seq F2
gcatggcgcggatttgacgaac

SEQ ID NO: 47 - DNA, 22 bp, Artificial Sequence
  Primer DDko seq F5
cattaaagagaccaagtacgtg

SEQ ID NO: 48 - DNA, 24 bp, Artificial Sequence
  Primer DDko seq F7
atatcctggtggtgtcgtcggcgt

SEQ ID NO: 49 - DNA, 22 bp, Artificial Sequence
  Primer DDko seq F9
tctttgtcaccaacgccctgcg

SEQ ID NO: 50 - DNA, 22 bp, Artificial Sequence
  Primer DDko seq R1
gcccaccgcgctcgccgccgcg

SEQ ID NO: 51 - DNA, 22 bp, Artificial Sequence
  Primer DDko seq R3
cccccaggatggcggcttcggc

SEQ ID NO: 52 - DNA, 22 bp, Artificial Sequence
  Primer DDko seq R7
gggccgacggcgataatcactt

SEQ ID NO: 53 - DNA, 22 bp, Artificial Sequence
  Primer DDko seq R10
ttcttcgatccactccttaacg

SEQ ID NO: 54 - DNA, 56 bp, Artificial Sequence
  Primer ChnA F
catcaattgactacgtagtcgtacgtgtaaggaggtttgaaatggaaaaaattatg

SEQ ID NO: 55 - DNA, 40 bp, Artificial Sequence
  Primer ChnA R
catgctagccccgggtatcttctactcattttttatttcg

SEQ ID NO: 56 - DNA, 22 bp, Artificial Squence
  Primer chnSeq F1
ctcaacagggtgtaagtgtagt

SEQ ID NO: 57 - DNA, 22 bp, Artificial Sequence
  Primer chnSeq R1
cgttttgatatagccaggatgt

SEQ ID NO: 58 - DNA, 35 bp, Artificial Sequence
  Primer Top ter F1
ctagaagtcaaaagcctccgaccggaggcttttga

SEQ ID NO: 59 - DNA, 60 bp, Artificial Sequence
  Primer Top ter F2
ctgctcgagttgctagcaagtttaaacaaaaaaaagcccgctcattaggcgggctgagct

SEQ ID NO: 60 - DNA, 37 bp, Artificial Sequence
  Primer Bot ter R1
cagcccgcctaatgagcgggcttttttttgtttaaac

SEQ ID NO: 61 - DNA, 50 bp, Artificial Sequence
  Primer Bot ter R2
ttgctagcaactcgagcagtcaaaagcctccggtcggaggcttttgactt

SEQ ID NO: 62 - DNA, 22 bp, Artificial Sequence
  Primer pCL1925 vec F
cggtatcatcaacaggcttacc

SEQ ID NO: 63 - DNA, 22 bp, Artificial Sequence
  Primer pCL1925 vec R1
agggttttcccagtcacgacgt

SEQ ID NO: 64 - DNA, 22 bp, Artificial Sequence
  Primer pCL1925 vec R2
cgcaatagttggcgaagtaatc

SEQ ID NO: 65 - DNA, 20 bp, Artificial Sequence
  Primer N84 Seq R2
gcatcgagattatcgggatg

SEQ ID NO: 66 - DNA, 208 bp, Escherichia coli
atcgcccgcattcttgccgcatcttcccccggcgtcacaccgaagtaacgtttaaactcacggctgtgtaggctggagctgcttcgaagttcctatactttctagagaataggaacttcggaataggaactaaggaggatattcatatgattacgttggatgtcagccgccgtatatacgaagccgcccgctaagctttttacgcctc

SEQ ID NO: 67 - DNA, 42 bp, Artificial Squence
  Promoter 1.6GI Variant
gcccttgacaatgccacatcctgagcaaataattcaaccact

SEQ ID NO: 68 - DNA, 42 bp, Artificial Sequence
  Promoter 1.5 GI
gcccttgactatgccacatcctgagcaaataattcaaccact

SEQ ID NO: 69 - DNA, 3240 bp, Klebsiella oxytoca
ggcgcggtccgccaggcggtcacctccgcgcgcgaaatcggcaaaaccgtccttgcgaccctcggtgctgaaccgaaaaacgatcgcccgtcctacatctgatacccacgaggctgattcatgagatcgaaaagatttgaagcactggcgaaacgccctgtgaatcaggacggcttcgttaaggagtggatcgaagaaggctttatcgcgatggaaagcccgaacgacccaaaaccgtcgattaaaatcgttaacggcgcggtgaccgagctggacgggaaaccggtaagcgattttgacctgatcgaccactttatcgcccgctacggtatcaacctgaaccgcgccgaagaagtgatggcgatggattcggtcaagctggccaacatgctgtgcgatccgaacgttaaacgcagcgaaatcgtcccgctgaccaccgcgatgacgccggcgaaaattgtcgaagtggtttcgcatatgaacgtcgtcgagatgatgatggcgatgcagaaaatgcgcgcccgccgcaccccgtcccagcaggcgcacgtcaccaacgtcaaagataacccggtacagattgccgccgacgccgccgaaggggcatggcgcggatttgacgaacaggaaaccaccgttgcggtagcgcgctatgcgccgttcaacgccatcgcgctgctggtgggctcgcaggtaggccgtccgggcgtgctgacgcagtgctcgctggaagaagccaccgagctgaagctcggcatgctgggccacacctgctacgccgaaaccatctccgtctacggcaccgagccggtctttaccgacggcgacgacacgccgtggtcgaagggcttcctcgcctcgtcctacgcctctcgcgggctgaaaatgcgctttacctccggctccggctcggaagtgcagatgggctacgccgaaggcaaatccatgctttatctggaagcgcgctgcatctacatcaccaaagccgcgggcgtacagggtctgcaaaacggttccgtaagctgcatcggcgtgccgtctgcggtgccttccggcattcgcgcggtgctggcggaaaacctgatctgttcgtcgctggatctggagtgcgcctccagcaacgaccagaccttcacccactccgatatgcgtcgtaccgcgcgcctgctgatgcagttcctgccgggcaccgactttatctcctccggttattccgcggtgccgaactacgacaacatgttcgccggctccaacgaagatgccgaagactttgacgactacaacgtcatccagcgcgacctgaaggtggacggcggtttgcgtccggttcgcgaagaggacgtcatcgccatccgtaacaaagccgcccgcgcgctgcaggccgtgtttgccggaatggggctgccgccgattaccgatgaagaagttgaagccgcgacctacgcccacggttcgaaagatatgccggagcgcaacatcgtcgaagacatcaagttcgcccaggaaatcatcaataaaaaccgcaacggtctggaagtggtgaaagcgctggcgcagggcggattcaccgacgtggcccaggacatgctcaacatccagaaagctaagctgaccggggactacctgcatacctccgcgattatcgtcggcgacgggcaggtgctgtcagccgtcaacgacgtcaacgactatgccggtccggcaacgggctatcgcctgcagggcgaacgctgggaagagattaaaaacatccctggcgctcttgatcccaacgagattgattaaggggtgagaaatggaaattaatgaaaaattgctgcgccagataattgaagacgtgctcagcgagatgaagggcagcgataaaccggtctcgtttaatgcgccggcggcctccgcggcgccccaggccacgccgcccgccggcgacggcttcctgacggaagtgggcgaagcgcgtcagggaacccagcaggacgaagtgattatcgccgtcggcccggctttcggcctggcgcagaccgtcaatatcgtcggcatcccgcataagagcattttgcgcgaagtcattgccggtattgaagaagaaggcattaaggcgcgcgtgattcgctgctttaaatcctccgacgtggccttcgtcgccgttgaaggtaatcgcctgagcggctccggcatctctatcggcatccagtcgaaaggcaccacggtgatccaccagcaggggctgccgccgctctctaacctggagctgttcccgcaggcgccgctgctgaccctggaaacctatcgccagatcggcaaaaacgccgcccgctatgcgaaacgcgaatcgccgcagccggtcccgacgctgaatgaccagatggcgcggccgaagtaccaggcgaaatcggccattttgcacattaaagagaccaagtacgtggtgacgggcaaaaacccgcaggaactgcgcgtggcgctttgataaaggataactccatgaataccgacgcaattgaatcgatggtacgcgacgtattgagccgcatgaacagcctgcagggcgaggcgcctgcggcggctccggcggctggcggcgcgtcccgtagcgccagggtcagcgactacccgctggcgaacaagcacccggaatgggtgaaaaccgccaccaataaaacgctggacgactttacgctggaaaacgtgctgagcaataaagtcaccgcccaggatatgcgtattaccccggaaaccctgcgcttacaggcttctattgccaaagacgcgggccgcgaccggctggcgatgaacttcgagcgcgccgccgagctgaccgcggtaccggacgatcgcattcttgaaatctacaacgccctccgcccctatcgctcgacgaaagaggagctgctggcgatcgccgacgatctcgaaagccgctatcaggcgaagatttgcgccgctttcgttcgcgaagcggccacgctgtacgtcgagcgtaaaaaactcaaaggcgacgattaacttctctaagtaattcgagatgcattgaggcggcaagtgagtgacaaattcgtctggaacgaatttgaacagccataggctggctttagtgagggacagggatgtccctcataaccccgatgagcttactgtagtaagtgattcgggtgaaagaacgcagccaacaaaaaggcagtttgaagtacgacgagaaaaggggcatgtgatgcgatatatagctggcattgatatcgg

SEQ ID NO: 70 - DNA, 2640 bp, Klebsiella oxytoca
acgtcgagcgtaaaaaactcaaaggcgacgattaacttctctaagtaattcgagatgcattgaggcggcaagtgagtgacaaattcgtctggaacgaatttgaacagccataggctggctttagtgagggacagggatgtccctcataaccccgatgagcttactgtagtaagtgattcgggtgaaagaacgcagccaacaaaaaggcagtttgaagtacgacgagaaaaggggcatgtgatgcgatatatagctggcattgatatcggcaactcatcgacggaagtcgccctggcgaccctggatgaggctggcgcgctgacgatcacccacagcgcgctggcggaaaccaccggaatcaaaggcacgttgcgtaacgtgttcgggattcaggaggcgctcgccctcgtcgccagaggcgccgggatcgccgtcagcgatatttcgctcatccgcatcaacgaagcgacgccggtgattggcgatgtggcgatggaaaccattaccgaaaccatcatcaccgaatcgaccatgatcggccataacccgaaaacgcccggcggcgcggggcttggcacaggcatcaccattacgccgcaggagctgctaacccgcccggcggacgcgccctatatcctggtggtgtcgtcggcgttcgattttgccgatatcgccagcgtgattaacgcttccctgcgcgccgggtatcagattaccggcgtcattttacagcgcgacgatggcgtgctggtcagcaaccggctggaaaaaccgctgccgatcgttgacgaagtgctgtacatcgaccgcattccgctggggatgctggcggcgattgaggtcgccgttccggggaaggtcatcgaaaccctctctaacccttacggcatcgccaccgtctttaacctcagccccgaggagacgaagaacatcgtcccgatggcccgggcgctgattggcaaccgttccgccgtggtggtcaaaacgccatccggcgacgtcaaagcgcgcgcgatacccgccggtaatcttgagctgctggcccagggccgtagcgtgcgcgtggatgtggccgccggcgccgaagccatcatgaaagcggtcgacggctgcggcaggctcgataacgtcaccggcgaatccggcaccaatatcggcggcatgctggaacacgtgcgccagaccatggccgagctgaccaacaagccgagcagcgaaatatttattcaggacctgctggccgttgatacctcggtaccggtgagcgttaccggcggtctggccggggagttctcgctggagcaggccgtgggcatcgcctcgatggtgaaatcggatcgcctgcagatggcaatgatcgcccgcgaaatcgagcagaagctcaatatcgacgtgcagatcggcggcgcagaggccgaagccgccatcctgggggcgctgaccacgccgggcaccacccgaccgctggcgatcctcgacctcggcgcgggctccaccgatgcctccatcatcaaccccaaaggcgacatcatcgccacccatctcgccggcgcaggcgacatggtgacgatgattattgcccgcgagctggggctggaagaccgctatctggcggaagagatcaagaagtacccgctggctaaggtggaaagcctgttccatttacgccacgaggacggcagcgtgcagttcttctccacgccgctgccgcccgccgtgttcgcccgcgtctgcgtggtgaaagcggacgaactggtgccgctgcccggcgatttagcgctggaaaaagtgcgcgccattcgccgcagcgccaaagagcgggtctttgtcaccaacgccctgcgcgcgctgcgtcaggtcagccccaccggcaacattcgcgatattccgttcgtggtgctggtcggcggttcgtcgctggatttcgaagtcccgcagctggtcaccgatgcgctggcgcactaccgcctggttgccggacggggaaatattcgcggcagcgagggcccccgaaacgcggtggccaccggcctgattctctcctggcataaggagtttgcgcatgaacggtaatcacagcgccccggccatcgcgatcgccgtcatcgacggctgcgacggcctgtggcgcgaagtgctgctgggtatcgaagaggaaggtatccctttccggctccagcatcacccggccggagaggtcgtggacagcgcctggcaggcggcgcgcagctcgccgctgctggtgggcatcgcctgcgaccgccatatgctggtcgtgcactacaagaatttacccgcatcggcgccgctttttacgctgatgcatcatcaggacagtcaggcccatcgcaacaccggtaataacgcggcacggctggtcaaggggatccctttccgggatctgaatagcgaagcaacaggagaacagcaggatgaataacgcactgggactggttgaaacaaaagggttagtgggcgccattgaggccgccgatgcgatggtgaaatccgccaacgtgcagctggtcggctacgaaaaaattggctcgggcctcgtcaccgtgatggtgcgcggcgacgtcggcgcggtcaaagcggcggtagacgcgggcagcgcggcggcgagcgcggtgggcgaag

SEQ ID NO: 71 - DNA, 756 bp, Acinetobacter sp.
atggaaaaaattatgtcaaataaattcaacaataaagtcgctttaattactggcgctggttcaggtattggtaaaagcaccgcactgcttttggctcaacagggtgtaagtgtagtggtttcagatattaacctggaagcagcacagaaagttgtggacgaaattgtcgctttaggcgggaaagcggctgcgaataaggccaatactgctgagcctgaagacatgaaagctgcagtcgagtttgcggtcagcacttttggtgcactgcatttggccttcaataatgcgggaattctgggtgaagttaactccaccgaagaattgagcattgaaggatggcgtcgtgtgattgatgtgaacttgaatgcggttttctacagcatgcattatgaagttcctgcaatcttggccgcagggggcggagcgattgtcaataccgcttctattgcaggcttgatcgggattcaaaatatttcaggctatgtcgctgcaaaacatggcgtaacgggtctaacgaaagcggcggcattggaatatgcagataaagggattcgcattaattcagtacatcctggctatatcaaaacgcctttgattgcagaatttgaagaagcagaaatggtaaaactacatccgattggtcgtttgggacagccggaagaagttgctcaggttgttgccttcctactttctgatgatgcttcatttgtgaccggtagtcagtatgtggtcgatggtgcatatacctcgaaataa

SEQ ID NO: 72 - protein, 251 aa, Acinetobacter sp.
MEKIMSNKFNNKVALITGAGSGIGKSTALLLAQQGVSVVVSDINLEAAQKVVDEIVALGGKAAANKANTAEPEDMKAAVEFAVSTFGALHLAFNNAGILGEVNSTEELSIEGWRRVIDVNLNAVFYSMHYEVPAILAAGGGAIVNTASIAGLIGIQNISGYVAAKHGVTGLTKAAALEYADKGIRINSVHPGYIKTPLIAEFEEAEMVKLHPIGRLGQPEEVAQVVAFLLSDDASFVTGSQYVVDGAYTSK

SEQ ID NO: 73 - DNA, 17417 bp, Acinetobacter sp.
ctagcatttacgcgtgaggtaggtgggtaggtctgtaatgtgaagatctacgaggaaatcggcgtcatgacgtgaggtccagcgaaccgtcttgcgtaatccgtcattcatggtgagtaacattgcccgtatttcgcgttcagtatatagcagaccagcatgattaacgagatcctgggtattttagtccggacacccaaagtcccatgcggtcgccagatccagtaagtcgactacgacttgctcatctgtagccaaccccgcaatcacttccacaattttcatcagtggaaccggattgaagaaatggaaacctgcgatacggccctgatgctgacacgcagatgcaattgaggtcacagatagtgaggatgtatttgaaaccagaatagtttcttcagccacaatcctttcaagctgtttaaacaaagtttgcttgatttccagattttcaataattgcttctacgaccagatcaacgccagcaacctcttcaatgctttccaagataatcaatcgggctaaggtatccacaagctgctgttcggttaactttcctttagcagctagtttgtgcaaggttacttttaatttttccaagccttgctcagcagcgccgggtttagcatcaaataaacggacctcaacacccgcctgtgctgcaatttgcgcaatacccattcccattacgcctgtgccaatcaaggccattttttgaatcgtcatgacttattttccttgatattgagggcttcgcttttcgaaaaaggcattgacgccttctttttgatcttgtgtatcaaataaaatttggaaggctttacgctctaatgccaaagcaccatcgagtggcatattggcacctagtgttgtgacttctttgatctgttcaacggcaatcggtgagagttgggcaatctgtgtcgcaatttcaaccgctttagcaagggtttgatcatcctcaaccacttcggaaaccaaccccattttgtcagcttcttctgcagaaaagatctttcctgttaacactatttgcatggctttaaacttccctaccgcacgcagtaagcgttgggtaccaccagcacctggcatcagccccaatttgacttcaggctgaccaaactgggctgattttccggcaataatgatgtctgcatgcattgcaagttcacacccaccacccaatgcatatccattcacagcagccacaatcggtttagggcaatcaataatggcccgccagtactgttccgtatggcgtaaatacatgtctacggtttttgcagtggtgaagtcccggatatccgcacctgctgcaaatactttttcaccaccagtaatgacaattgcgcggactgtatcagatgcagcgagctgctcaaacattgctgcgagctgttggcgcagttccagattcaatgcatttctagtatctggacgatgtagttcaacaatggccacaccattactttgaatatctaaattcaatatttcattttccataacaacctacatgtttcgcatagcggtttatttaaaccaaatatacctgtttttttgcaacaataaagcccacaggaacatagttttaaattaaaaattggctaaaaatatttaaaaaacacaaataaaataccgcacagcggtatttgatatcaatattattgcatttatttttccattctgtcatattattttcattccaaagcattagatcacccctgcatgaagcagagatggctaaatttacctatctaatacaagggcttaaaaatgattcgcgatcaagacacattaaatcagctggttgacatgatccgtcagtttgtcgatggcgttcttattcccaatgaagaaattgttgcggaaaccgatgaaattccagctgaaatcgtgcagcaaatgaaagaactgggtctttttggtctcaccattcctgaggaatatgagggtcttggcctgaccatggaggaagaggtttacattgcatttgaactgggacgtacctctcctgctttccgttcactgatcggcactaacaatgggatcggttcatcaggcttaattattgatggctccgaagagcagaaacagtattttttgccacgtctggcaagtggtgaaattattggttcattctgtttaactgaacctgattccggttcagatgctgcctctttaaaaaccacagcggtgaaagatggtgatcattacattttaaatggcactaagcgttacatcaccaatgcaccgcatgcgggtgtctttactgtcatggcacgtaccagtaccgaaattaaaggtacaggtggaatttcagcctttatcgtggacagtaaaactcctggtatttccttgggtaaacgtgataagaagatgggccaaaaaggtgcacatacctgtgatgtgatttttgaaaactgtcgtattcctgcatctgcactcattggtggtgttgaaggtgtaggttttaaaactgcaatgaaggtacttgataaaggccgtattcatattgctgcattaagtgtaggtgctgctacgcgtatgctggaagattccctacaatatgccgttgagcgcaaacagtttggtcaagcgattgcgaacttccagttgattcaaggtatgttagccgattctaaagctgaaatttacgcagcaaaatgtatggtattagatgctgcccgacttcgtgatgctggacagaatgtcagcacggaagcatcttgtgccaagatgtttgccactgaaatgtgtggccgtgtcgcagatcgtggcgtacagatccatggtggtgcgggttatatcagtgaatatgctattgagcgtttttaccgtgatgtacgtttattccgtttgtatgaaggtacaacgcaaatccaacaggtcattattgcccgcaatatgatccgtgaagcgactcaataattgtataacaggtattgagtgtatctaaaaggacgggattagtgatttaagctataacttgaatactaatcctgactttttgatggcaaggctataaaacctcctagctcattttatctctaagctaatcacagctgaaagatattttcagtcttcatccttaccagacagttcacaatacaaaattggattttatgaatatgcaagaacaagaaatcgaacgcgaatcaatggagtttgacgtcgtgattgtcggcgcaggaccggccggtctttctgcagcgatcaagatccgtcaacttgcaattgaaaacaacctgaacgatctgtcggtttgtgtggtggaaaaaggctctgaagtcggtgcgcacatcttgtccggtgcggtactggaaccacgtgccatgaatgagctgttcccgaactggaaggaagaaggtgcacctttaaatgttccagtgaccgaagacaagacctatttcctgctctcggatgaaaaatcacaagaagcgccacactggatggtgcctaaaaccatgcataacgatggcaactatgttatctcgctcggcaacgtagtgcgctggttgggtcaaaaagcggaagagctggaagtatctattttcccgggctttgccgctgctgaaattctgtaccatgcagatggttcggtgaaaggcattcaaaccggtgacatgggcattggcaaggatggcgaaccgacccataactttactccgggctatgaactgcatgccaaatacaccctgtttgctgaaggctgccgtggccacctcggcaagcgtttaattgccaaatacaacctcgataaagattcagatccacaacattacggtatcggtatcaaagagctgtgggaaatcgacccggcgaaacacaagccaggtctggtgatgcacggtgccggctggccattgtctgaaaccggttcttcaggcggctggtggttgtatcatgcggaaaacaatcaggtgactttgggcatgatcgtcgatctgtcttacaccaacccgcatatgtatccgtttatggaaatgcagcgctggaaaacccatccgctgatcaagcagtatctggaaggtggcaaacgtatttcttatggcgcgcgtgcggtaaccaaaggcggctttaactcgctaccgaaatttaccttcccgggcggatcgctgattggtgacgatgccggcttcctgaactttgccaaaatcaagggctcacataccgcgatgaaatccggcatgctctgcggtgaagcagtgtttgaagccattgctgccggtgtggaaaaaggtggtgaccttgcggttgcgcgtgtgacggaaggcgaagacttgtttgccaaaaaactgacttcttacaccgacaagttcaataatagctggctgaaagaagagctgtacaactcgcgtaactttggcccggccatgcacaagtttggtcagtggctcggtggtgcgtttaactttatcgaccagaacgtgtttaaggtgccgtttaccctgcatgacctggtgacggatttcggtgcgctgaaaaccgtcgatgcggtgaacttcaagccgaattatccaaaaccggatggcaaactgacctttgaccgtctgtcttcggtgtttgtatccaacacggtgcatgaagaaaaccagccagcgcatttaaaactgactgacacttcgattccggtgaatgtcaacctgccaaaatgggatgaaccggcgcagcgctactgccccgcgggtgtatacgaaatcatggaaaatgatgacggttcgaaacgcttccagatcaatgcagccaactgtgtgcactgcaagacctgtgacatcaaggatccttcacagaacatcacctgggtaacaccggaaggtggtggtggtccaaactatccgaatatgtaagtctaatcacttcaaggaagaggtttcccatttcccttctttctagcagatgaagaagcttgcaactaaaagagattgtttggatcagttacccaaaatcgttgaaaagattttaactcttcgatttttattttttaggtaatcctagccctctcgggggctaggattaaaaattttaagttattccaacacgaatgacaaattgttcaatgcaaaataaaaacatacaatatataaatatattttttaattaaaacataagattacaataaaataagaatttttatttggagtttgttttttttctacaatgatcattatgtacaatttttaggttcaccccatccaagccttgtgattgcattcctgcgattctttattcaatgaataagcaatgctattaatcagcaatgaataaccagcactgcagattttgaataaattcacatgtcgtaatggagattatcatgtcacaaaaaatggattttgatgctatcgtgattggtggtggttttggcggactttatgcagtcaaaaaattaagagacgagctcgaacttaaggttcaggcttttgataaagccacggatgtcgcaggtacttggtactggaaccgttacccaggtgcattgtcggatacagaaacccacctctactgctattcttgggataaagaattactacaatcgctagaaatcaagaaaaaatatgtgcaaggccctgatgtacgcaagtatttacagcaagtggctgaaaagcatgatttaaagaagagctatcaattcaataccgcggttcaatcggctcattacaacgaagcagatgccttgtgggaagtcaccactgaatatggtgataagtacacggcgcgtttcctcatcactgctttaggcttattgtctgcgcctaacttgccaaacatcaaaggcattaatcagtttaaaggtgagctgcatcataccagccgctggccagatgacgtaagttttgaaggtaaacgtgtcggcgtgattggtacgggttccaccggtgttcaggttattacggctgtggcacctctggctaaacacctcactgtcttccagcgttctgcacaatacagcgttccaattggcaatgatccactgtctgaagaagatgttaaaaagatcaaagacaattatgacaaaatttgggatggtgtatggaattcagcccttgcctttggcctgaatgaaagcacagtgccagcaatgagcgtatcagctgaagaacgcaaggcagtttttgaaaaggcatggcaaacaggtggcggtttccgtttcatgtttgaaactttcggtgatattgccaccaatatggaagccaatatcgaagcgcaaaatttcattaagggtaaaattgctgaaatcgtcaaagatccagccattgcacagaagcttatgccacaggatttgtatgcaaaacgtccgttgtgtgacagtggttactacaacacctttaaccgtgacaatgtccgtttagaagatgtgaaagccaatccgattgttgaaattaccgaaaacggtgtgaaactcgaaaatggcgatttcgttgaattagacatgctgatatgtgccacaggttttgatgccgtcgatggcaactatgtgcgcatggacattcaaggtaaaaacggcttggccatgaaagactactggaaagaaggtccgtcgagctatatgggtgtcaccgtaaataactatccaaacatgttcatggtgcttggaccgaatggcccgtttaccaacctgccgccatcaattgaatcacaggtggaatggatcagtgataccattcaatacacggttgaaaacaatgttgaatccattgaagcgacaaaagaagcggaagaacaatggactcaaacttgcgccaatattgcggaaatgaccttattccctaaagcgcaatcctggatttttggtgcgaatatcccgggcaagaaaaacacggtttacttctatctcggtggtttaaaagaatatcgcagtgcgctagccaactgcaaaaaccatgcctatgaaggttttgatattcaattacaacgttcagatatcaagcaacctgccaatgcctaaatatatggggggcatcccccatattccattttgtttaacatcagtcatatgccagggatgtcttatcatgaactatccaaatatacctttatatatcaacggtgagtttctagatcataccaatagagacgtcaaagaagtttttaatccagtgaaccatgaatgtattggactcatggcctgtgcatcacaagcagacctggactacgcacttgaaagttcacaacaggcttttctaaggtggaaaaaaacttctcctatcacccgtagtgaaatcctcagaacctttgcgaaactagcgcgtgaaaaagcagcagaaatcgggcgcaatattacccttgatcaaggtaagcccctgaaagaagccattgcagaagtcactgtctgtgcagaacatgcagaatggcatgcagaagaatgccgacgcatttatggccgtgttattccaccgcgtaacccaaatgtacagcaactagtagtcagagaaccgctgggcgtatgtctggcattttcaccgtggaatttcccgtttaatcaggcaattcgtaaaatttctgctgcaattgctgccggctgcaccatcattgtgaaaggttctggcgacacaccaagcgcggtatatgcgattgcccagctatttcatgaggcgggtttgccgaatggtgtgctgaatgtgatttggggtgactcaaacttcatttctgattacatgatcaaatcgccgatcatccaaaagatttcattcacaggctcaaccccggtgggtaaaaaattagcctcgcaagcgagtctgtatatgaagccttgcaccatggaattgggtggtcatgcaccggtcatcgtctgtgatgatgctgatattgatgccgctgttgaacatctggtcggttataaattccgtaatgcaggacaggtctgtgtatcaccaacccgtttttatgtgcaggaaggtatttataaggaattttctgagaaagtggtgttaagagccaaacagatcaaagtgggttgtggcttagacgcatcctcagatatgggaccattggctcaagctcgccgcatgcatgcaatgcaacaaattgttgaagatgcggttcataaaggctcaaaattactgcttggcggaaataaaatttctgacaaaggcaatttttttgaaccaacggtactcggtgacttgtgcaatgacacccagtttatgaatgacgagccatttggtccgatcattggtttgataccttttgacacaatagaccatgtcctggaagaagcaaatcgattaccatttggattagcctcttacgcttttaccacatccagcaaaaatgcgcatcaaatctcatacggactggaggctggcatggtttcgattaaccacatgggattggcgctcgctgaaacaccttttggtggtattaaggatagcggttttggtagtgaagggggtatcgaaacctttgacggttacctcagaaccaaatttattacgcaactcaattagaaatggatcttggtgtgcgtaggcacaccaattctcttttgactttaaggatgaaagttaaatgagcacagacaaagcaaatacgctgatcaaacccgaagatgtcgtgttatggattccgggtaatgtcacaattgacagcatgaatgccggttgggaaaacattgcaatcagagggtacgaatataccaacctcgatgtgcatattcctgccatgcgtgactacatgatcgtcaactataaaaaaagtgcggcggaaatgcgtagaaaaggcgatgcctcttgggatacccaagtggttaagccgggttatgtctccttgttgacctgtggtgaagattcccgctgggcgtggaatgaccatattgccgtcacccatgtctacatttcgcatgactccatcacctcaatggcgaataaggtgtttgattatgatatcgcttcgatccgaatcagagacgaagtcggtgtggaagatcatgttttacctgctctgacttcacttttagaactagaattaaagcaaggtggtttaggtggaaacctgtatttagagagcattaaaaaccagatcgccctgcatttactccgtcagtatgccaaattagattttaaggaaggacagtgccgttctggttttactcccctacaacgcagactgttattagaatttatcaatgaaaacatgagcattaaaattaccctcgaagatttagcgggattagtcaagatgagcgtgcctcatttaatgagaaaatttaaagtcgattttggtaattcccctgctgcctacatcatgaatctcagggtgcaatttgctaaacgtttgctcacttcaaaaaaagaaattccactgaaagtgattgccagtgaagccggtttttgcgatcagagccatatgacccgagtatttcaaaaattttttgggaaaacacccatcgaaatcagacaggaacacaccaatctcgtgtctgaaaattcagtctcctctattgttttttgagtactaagagccacgcaagaacctgattttcaataaagcatccactgaaaaccagtgtggacttacatgcattatttatgcaaaataacaaatgtcatgtgagtatcaagatatactttctatcgctatcaagaacttgccagtacaggcaatatggatgcactcatcaaccagagtcgcagaactccaaatttaaaaaaccgagtggatgagcaaactgaataagctgttgttgattttgcaatccaatatccagcttatggtcagcatcggaccagtaatgagctacgtcagattggcatcttcgtatctggcagcggtgtgcgctctatctggcttagacacaatcttgagaatttcaaaaagcgattaaaggcacttgaaattaaagttgctcaagaaggcattcagttgaatgatcagcagattgccgcattagaacgtaaacatgaagatgatgttgcttgtggtgaaattgaaacacatcatccaggttaccttggagcacaagatactttttatgtcggaaatctaaaaggtgttgggcatatttatcagcaaacttttattgatacttatagcaaagtggttcactgcaagctgtacacaaccaagacaccaatcacagccgcagatttattgaatgaccgcgtgttaccattctatgagtcacaaggattgccaatgcttcgcattttgaccgacagaggcaccgaatattgcggtaaagttgaacatcacgattatgagctttatttggctctgaatgatattgatcacactaaaactaaagcagcatcaccacaaacaaatgggatctgtgagcgcttccataagacgatcttgcaggagttttatcagattacttttcgaaagaaactctatagctcattagaagagttacagcttgatctagacggttggctgaaattctataatactgaacgaacccatcagggtaaggtgtgtaatggcagatgagcagcattgctgcgcaagattgcaacattacttgatggaaaacgtatttgggctgaaaagaatttagttcaaatttaacctgacagtcttaagcaaatatcggtaactatcagatcaggtttgagataccgtctgaaacgtcaagtaaatgattgagaattcatgctcaataatctgcttgataaggctgttggtgtttgagcacaccataacaaagatgaatcaacttcctcatcgcggctccaatcgctatcatcttggttttaccattcgccaataaacgttcattcattgccctgatgtgagggttatgccgagttgcgacaatggctgccatatataaaccagcacgtattttggaagagcccgctttggataaacggcttctgccatgaatggaactacccgattgcttttgaatggggaccaaaccgacaaaggcagccgcttgactagccctttcaaaagtatggctgcgcaagaaactgagcattaataaactggttcgatctgcaatggctggaatactgctgagcagttctttatcattttttaaatcaggattctgattaatgtgatcatcaatttgctggtcgataccctgaatgtgtttgtttaactgttcaatactcttgtggatagactgaagtacaggttccatcgtgaaggtcgactctgctttttccaaacgattcttttcacgttgtaaatcttcacaaagaatagctcttctatccagcaaagcattcagcaattgaatatgtttaggtaaaggttgccaaaaatgtagatcggcagtcatcgcaaatcgagctaggacctcactatccaccttgtctgttttattcagcttagacatactctgagcaaaatatcgagctctggcaggattggttacacagacttgatagcccgcatcaaataaatatttaaccaagagttcatgataaatagatgttgcttccattaaaataatggtctgcgtagaagttgcagcatgctgctttagccaggtttgaagttgctcaaaaccttttggtgtatttgaaaaagttttggttttctttttatttgcagaattttctaaaattaaacagcaatcaattttagctttagcaacatcaataccaagataaaacataatctttacctgctttatttatccaattattgttttagcataaccaccgtcttttcttgtgaatgcagcatcaaagtgcttgttaccgtccagagttgtgcaagtggttagggcaaattacaggttttatctcaaactctaactttatgttttgctagtacacgaaactctgcaatttgcaatatagtgatagctaatcactatgaatggtaagatacaagctagtacacataagaagatattacttcttctcaggcagattcgcagcaaagaaaaattttcccttacaacaatagataaaagaaaagagggtatcacccctctttcctctttatatgggggtatcttctactcattttttatttcgaggtatatgcaccatcgaccacatactgactaccggtcacaaatgaagcatcatcagaaagtaggaaggcaacaacctgagcaacttcttccggctgtcccaaacgaccaatcggatgtagttttaccatttctgcttcttcaaattctgcaatcaaaggcgttttgatatagccaggatgtactgaattaatgcgaatccctttatctgcatattccaatgccgccgctttcgttagacccgttacgccatgttttgcagcgacatagcctgaaatattttgaatcccgatcaagcctgcaatagaagcggtattgacaatcgctccgccccctgcggccaagattgcaggaacttcataatgcatgctgtagaaaaccgcattcaagttcacatcaatcacacgacgccatccttcaatgctcaattcttcggtggagttaacttcacccagaattcccgcattattgaaggccaaatgcagtgcaccaaaagtgctgaccgcaaactcgactgcagctttcatgtcttcaggctcagcagtattggccttattcgcagccgctttcccgcctaaagcgacaatttcgtccacaactttctgtgctgcttccaggttaatatctgaaaccactacacttacaccctgttgagccaaaagcagtgcggtgcttttaccaatacctgaaccagcgccagtaattaaagcgactttattgttgaatttatttgacataattttttccatttcaaattttaagcatcaaagcttgtttcatattttaagattcaagaaaccagatccggtagatgactcgtctgccaagcgacaacccgtctgatatcaggcttgcgattcaccctgtagacggttttcattcctaaattctgtatttccaagttatataaacaaaagtgctaatctatggggaattcccaggatccaaacaaatagaatgccatgaaagcatcttttgccaagcgctgtgctgtatgtttcctagacaaaccaccaacgataactgcaactttttgaactccttacaatttccttattttctttccccttcatcgcataaaaatagtttttgcattcacaacaaaatcagcatgaatagtttttaaactcactgtacatattttctatattgatgaccaagctggatattgaattgcaaaattctatacagcctgttcaacatgatcgatttagaaggcatacagtaaacgtgactgaagtccagaaatttccaagccattttcaacattcacatcttgtcgccattgtaataatagctgcagattcggcttgatattggtagaagcagaaacgacaaaggtatcttttctatcactgccacgttcagtgacaccattcaccttttctttaccgccatcggtatgtctccaggtgacagccaaattggatttatcggtcactttatagagtgcggagaaatctgtctggaaaaaaacctctttctcaatgttggtatatttttgctcgctataaagttcaaactgccccaccccctcaagcgcaaatttatcagttaaagcatggtaataaccggcctgaacattatattgatagcgatcattactgatggcaaaacccttcgtttcattactgccggtaggtacggtcaaaaaaccaccgaaaccaaaatagcgccctttttcagcatcatgcaatggccaggcgataccacccacaattaaatcaccgacacccgagatatcatcagcgccattcatcttttgcttggcaaaaggcaagaggaattgaggatctacaatccaatcccctacttcaataaaacgaacgtaacgcaatattcccaaatcaatgcttaaatcgagatcatcagcgactttatcaccatttgcatacgccttatccgcttccgtatgctggtaataggcaaccgctaagttggttccccctggaagtgcttgataatccccggcatcagaactcacccctgcggcttgcaggtccaaagcggcagttaaagcaaagaccaaagcagctattttttgatttgaacgatgatagaaatagtttttcatttgtttcatttttaactctccgttgttttgactcatttttttaaaatgagtcttcctagcacaaagaccactcaggtctttgcgcaatttcttgattttgatttgggtattaaatatggaaaaacgttgggtgatcagttttcgtgcataagcacaatacgcccgatgacgttgccatctttcaagtctccaaatgcggaattgatctgcgaaattggcagttttttcacgggaatggctgacatgtgggtttctttcaccagctccaccagctctcttaattcctctaccgtccctacataactgccctggattgtgagtggtctcattggaatcaccggaatggaaagcttaatttctccccccatcaatccgcagatcacaatatgcccaccacgtgcagcactcgccaaggcaaggctcaatgttggattactgccaaccagatcaaggatcagacgtgcaccaccgtcagttgcctgaatcagctgttgagcagcatcctcacttcggctattgatgaccgataatgcaccggcagcacgtgctgcttccagtttgctgtcatcaatatcaactacgattgcgcctttggcttgcatagctttgagcaactcgagtgccatcagccctaaaccaccggcaccaatgatcaccaccggctcgctttgaatcaaatcaccgaattttttcagtgcactgtatgttgtcacgcctgcacatgccaaaggtgcagcttcagccagatccagacctgcaatatccaccagatatcgtggatgcggcacgatgatatattcggcaaaaccacccggcttggcgatgcctaactgttgcggtttggcacacaggttttcttcgccacgtttacagtagttgcattcaccgcaaccaatccatggatgaaccaagctgaccatgccgaccttgactgattccgcatctggaccgacagcaaccacctgacctgtaatttcatgacttaaggttaagggtggcttcagcccacgatctgcaagggataaacgcttgcccccacctagatcataataaccttcccataagtgtaaatccgtatggcatagacctgcggcttttacatggagtaaaacttcagtacctttcggttgcggaatttctttctcaacgtcttcgagtggttgtccatgatgcgtcacgcagtaacagtgcatgaatctctcctttgaaacaataaaatagacggccttgtagtgaacaaagtcttttattcactaagttttatacgccgtgtgggcactgatttatgctttaaaccactgcgcaattttcgctaattcttgatcagcttcacttgcacgcccagctaggaaaggaaaaacgtgctgcatgttgtccaccacagataaagtcacatcaacaccctctttttttgcaatatcagcaagacgtgttgcattgtctacaagtgattcaactgatccggcattgatatacaaacgtgggaaaacctgataattggcttttaacggattcgccaatggatttgccggatcaccatgttcacccaagaacatttgtgacatgcctttaagcagatccactgtaatcaaggcatcagtggcatcgttgctgatcagggtttcacctttgtgctccatatccagccaaggagagaatgcaatcactgctcctggcaactcaatcccttcatttcgtagattgagtacggttgatatcgccagattcccccccgcagaatcccctgcggtcagcatattttttgcagtaaagccacgctggagtagttctttatatactgctgtcacgtcctgaatttgtgccgggaagacatgttctggtgaacgtcggtaatcaaccacaaatgcggatacccctaaatacttggccaaatgccccaccagcttacggtgactggccgaagaaccgaccgcaaatccaccgccatgggtataaatgatgactttggataagtcagcatctttcggataaatccaaagaccttctacacctgccacaacatcgaatttataagacacttcttccggttccaatgtaggttgatgccattcatcaaacatactgcgaaagtcttcaatggtcatattcggattttcctgcatccgtcttgaccagttcgcatataaatcgaaaagaaattgagtattgctttgtgtgctattcattttaaaatccttgatttgatatttaaggaataaatcctagttttattccatgaagatataaaaacttgagtgccatcactcatggctagacactcagaagatccaaatctaaagagtggctttgcatcactggtttgatacaattttttgcatgactaagtaatctacggataatctaaccgtttcaaattagtattttaaaatgtaaaaaatacataccagcgaatgttttctgcaaaatcgcatcctgttcaatatagcttttgatcctacttattctcttttctattccagtccgttataaaaaagctttcattcattttcatgcaatcatgagctatgaatgttcttaaacattaaacgattgtgtgtatggctgacttgtacattcttgtacttatttttgtataaaatgatcaggctcatcaatttatgggaaaaattacaattcgggtacaatatctttcctgtttcatgaatctattcaactcattaaacttacgaccctcaactgcccaaaatcataggatctgccgatccacttgcagaattagcaatgctaaaacatgaactccaaagagttactaaaaaaagagcatattaaaaaaaagccgtggcatatttcgcaagccagttcaagtcaggtatgtctttattcagtacctcagttaaactttagattttcataacgatggttattctgcatggctaaatacgctaatcagcaaaaaactctccaaaagataggcacagaaacacatatcaaccataaaaaccatctcagacagtatatttacaagcctctaattcaccgcactcacacttctctgcaagcctttttaaataccctgtacaaagttctcagcctgatgaagcttcaccttggacttagctttcagttcagcctgtacttggtcagtttctgaattttcatttgcataaaactcctccaccacatccataccctcctcaatgtcagtttcaaaatgtgcattgtcatagccttgccgtgccatttgaatggcttattgaagattaatggcatcacgtaaagttaaatccacgtaatacacaggtgttcgatagctttgcgtcgtagactttctcgaagagtcaattgcagcggtaggcatgacagcaagccattcaatgccgcatggtaataactcagccgtgcggccaacgttcgtatgctgttaaaacccggttattctaa

SEQ ID NO: 74 - DNA, 1164 bp, Escherichia coli
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaa

SEQ ID NO: 75 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 76 - DNA, 1623 bp, Bacillus subtilis
atgtatttggcattccaggtgcaaaaattgatgcggtatttgacgctttacaagataaaggacctgaaattatcgttgcccggcacgaacaaaacgcagcaattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcatatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcactccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggtgcatttccagcaattgaaaaaatataaccgtacatctgcggtcgatttcggaaatatcgatatcgtgaaatatgcggaaagcttcggagcaactgcgttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctag

SEQ ID NO: 77 - protein, 540 aa, Bacillus subtilis
MYLAFQVQKLMRYLTLYKIKDLKLSLPGTNKTQQFMAQAVGRLTGKPGVVLVTSGPGASNLATGLLTANTEGDPVVALAGNVIRAYRLKRTHQSLDNAALFQPITKYSVEVQDVKNIPEAVTNAFRIASAGQAGAAFVSFPQDVVNEVTNTKNVRAVAAPKLGPAADDAISAAIAKIQTAKLPVVLVGMKGGRPEAIKAVRKLLKKVQLPFVETYQAAGTLSRDLEDQYFGRIGLFRNQPGDLLLEQADVVLTIGYDPIEYDPKFWNINGDRTIIHLDEIIADIDHAYQPDLELIGDIPSTINHIEHDAVKVEFAEREQKILSDLKQYMHEGEQVPADWKSDRAHPLEIVKELRNAVDDHVTVTCDIGSHSIWMSRYFRSYEPLTLMISNGMQTLGVALPWAIGASLVKPGEKVVSVSGDGGFLFSAMELETAVRLKAPIVHIVWNDSTYDMVHFQQLKKYNRTSAVDFGNIDIVKYAESFGATALRVESPDQLADVLRQGMNAEGPVIIDVPVDYSDNINLASDKLPKEFGELMKTKAL

SEQ ID NO: 78 - DNA, 1680 bp, Klebsiella terrigena
atggacaaaccgcgtcacgaacgtcaatgggcccacggtgccgacttaatcgtcagccagcttgaggcccagggcgtacgccaggtcttcggcatccccggtgccaaaatcgacaaggtgtttgattccctcctcgactcctcaatccgcattattccggtgcgccacgaggctaacgccgcctttatggccgcggcggtcgggcggattaccggtaaagcgggcgtcgcgctggtgacctccggtcccggctgctcaaacctgattaccggcatggccaccgccaatagcgaaggcgacccggtggtggcgctgggcggcgcggtgaagcgcgcggataaggccaagctggttcaccaaagcatggacaccgtggcgatgttcagcccggtcaccaaatacgccgtcgaggtgaccgcctccgacgcgctggccgaggtggtctccaacgcctttcgcgccgccgaacaggggcgtccggggagcgcgtttgtcagcctgccgcaggatatcgttgacggccccgccagcggcagcacgctgcccgccagcagagcgccgcagatgggcgccgcgccggatggcgccgttgacagcgtggcgcaggcgatcgccgcggcgaagaaccctatcttcctgctcgggctgatggccagccagccggaaaacagccgcgccctgcaccgccatgctggaaaaaagccatattccggtcaccagcacctatcaggcgccggggcggtaaatcaggataacttcgcccgcttcgccggccgggtaggcctgtttaataaccaggcgggcgatcgcctgctgcgtcaggcggacctgatcatctgcatcggctatagcccggttgagtacgaaccggcgatgtggaacagcggcacggcaaccctggtgcatatcgacgtgctgccggcctatgaagagcggaactacgtcccggatatcgagctggtgggcgacatcgccgccaccctcgagaagctggcccagcgcattgaacatcggctggtgttaactccgcaggcggcggacatcctcgccgaccgccagcgccagcgggagctgcttgaccgccgcggggcgcagctgaatcagtttgcgctccacccgctgcgcatcgtgcgggcgatgcaggatatcgtcaatagcgacgtcaccttgaccgtcgatatgggcagtttccatatctggattgcccgctacctctacagcttccgcgcccgccaggtgatgatctccaacggtcagcaaacgatgggcgtcgcgctgccgtgggcaatcggcgcgtggctggtcaatccgcagcgcaaggtggtctcggtatccggcgatggcggcttcctgcagtcgagcatggagctggagaccgccgtgcgcctgcacgccaatattctgcacatcatctgggtcgataacggctacaacatggtggcgattcaggaacagaagaaatatcagcgcctctccggcgtggagttcggcccggtcgatttcaaagtctacgccgaagcgttcggggcctgcgggtttgcggtagagagcgccgaggccctggagccgaccctgcgcgcggcgatggatgtcgacggcccggcggtggtcgccattccggtcgattaccgcgataaccctctgctgatgggccagctccatctcagccaaatactgtga

SEQ ID NO: 79 - protein, 559 aa, Klebsiella terrigena
MDKPRHERQWAHGADLIVSQLEAQGVRQVFGIPGAKIDKVFDSLLDSSIRIIPVRHEANAAFMAAAVGRITGKAGVALVTSGPGCSNLITGMATANSEGDPVVALGGAVKRADKAKLVHQSMDTVAMFSPVTKYAVEVTASDALAEVVSNAFRAAEQGRPGSAFVSLPQDIVDGPASGSTLPASRAPQMGAAPDGAVDSVAQAIAAAKNPIFLLGLMASQPENSRALHRHAGKKPYSGHQHLSGAGAVNQDNFARFAGRVGLFNNQAGDRLLRQADLIICIGYSPVEYEPAMWNSGTATLVHIDVLPAYEERNYVPDIELVGDIAATLEKLAQRIEHRLVLTPQAADILADRQRQRELLDRRGAQLNQFALHPLRIVRAMQDIVNSDVTLTVDMGSFHIWIARYLYSFRARQVMISNGQQTMGVALPWAIGAWLVNPQRKVVSVSGDGGFLQSSMELETAVRLHANILHIIWVDNGYNMVAIQEQKKYQRLSGVEFGPVDFKVYAEAFGACGFAVESAEALEPTLRAAMDVDGPAVVAIPVDYRDNPLLMGQLHLSQIL

SEQ ID NO: 80 - DNA, 768 bp, Bacillus subtilis
atgaaacgagaaagcaacattcaagtgctcagccgtggtcaaaaagatcagcctgtgagccagatttatcaagtatcaacaatgacttctctattagacggagtatatgacggagattttgaactgtcagagattccgaaatatggagacttcggtatcggaacctttaacaagcttgacggagagctgattgggtttgacggcgaattttaccgtcttcgctcagacggaaccgcgacaccggtccaaaatggagaccgttcaccgttctgttcatttacgttctttacaccggacatgacgcacaaaattgatgcgaaaatgacacgcgaagactttgaaaaagagatcaacagcatgctgccaagcagaaacttattttatgcaattcgcattgacggattgtttaaaaaggtgcagacaagaacagtagaacttcaagaaaaaccttacgtgccaatggttgaagcggtcaaaacacagccgattttcaacttcgacaacgtgagaggaacgattgtaggtttcttgacaccagcttatgcaaacggaatcgccgtttctggctatcacctgcacttcattgacgaaggacgcaattcaggcggacacgtttttgactatgtgcttgaggattgcacggttacgatttctcaaaaaatgaacatgaatctcagacttccgaacacagcggatttctttaatgcgaatctggataaccctgattttgcgaaagatatcgaaacaactgaaggaagccctgaataa

SEQ ID NO: 81 - protein, 255 aa, Bacillus subtilis
MKRESNIQVLSRGQKDQPVSQIYQVSTMTSLLDGVYDGDFELSEIPKYGDFGIGTFNKLDGELIGFDGEFYRLRSDGTATPVQNGDRSPFCSFTFFTPDMTHKIDAKMTREDFEKEINSMLPSRNLFYAIRIDGLFKKVQTRTVELQEKPYVPMVEAVKTQPIFNFDNVRGTIVGFLTPAYANGIAVSGYHLHFIDEGRNSGGHVFDYVLEDCTVTISQKMNMNLRLPNTADFFNANLDNPDFAKDIETTEGSPE

SEQ ID NO: 82 - DNA, 780 bp, Klebsiella terrigena
gtgaatcattatcctgaatgcacctgccaggagagcctgtgcgaaaccgtacgcggcttctccgcccaccaccctgatagcgttatctatcagacctctctgatgagcgcgctgctgagcggggtctatgagggtagcaccaccatcgccgacctgctgacccacggcgacttcggtctcggcacctttaacgaactcgatggcgaactgattgcctttagcagcgaggtctaccagctgcgcgctgacggcagcgcgcgtaaagcccgggcggatcaaaaaacgcccttcgcggtgatgacctggttcagaccgcagtaccgtaaaacctttgaccacccggtcagccgccagcagctgcacgacgttatcgaccagcaaatcccctccgataacctgttctgcgccctgcatattgatggtcactttcgccacgcccacacccgcaccgtgccgcggcagacgccgccctatcgggcgatgaccgacgtgctcgatgaccagccggttttccgcttcaaccagcgcaaggggacgctggtcggctttcgcaccccgcagcatatgcagggccttaacgttgccggctaccacgagcactttattaccgacgatcgccagggcggcggccatctgctggactaccagctcgatagcggcgtgctgaccttcggcgagatccacaagctgatgattgacctcccggccgacagcgctttcctgcaggccgacctgcatcctgacaatctcgatgccgctattcgtgcggtagaaaactaa

SEQ ID NO: 83 - protein, 259 aa, Klebsiella terrigena
MNHYPECTCQESLCETVRGFSAHHPDSVIYQTSLMSALLSGVYEGSTTIADLLTHGDFGLGTFNELDGELIAFSSEVYQLRADGSARKARADQKTPFAVMTWFRPQYRKTFDHPVSRQQLHDVIDQQIPSDNLFCALHIDGHFRHAHTRTVPRQTPPYRAMTDVLDDQPVFRFNQRKGTLVGFRTPQHMQGLNVAGYHEHFITDDRQGGGHLLDYQLDSGVLTFGEIHKLMIDLPADSAFLQADLHPDNLDAAIRAVEN

SEQ ID NO: 84 - DNA, 1053 bp, Bacillus cereus
atgaaagcactactttggcataatcaacgtgatgtacgagtagaagaagtaccagaaccaacagtaaaaccaggaacagtgaaaatcaaagttaaatggtgtggtatttgtgggacagacttgcatgaatatttagcagggcctatttttattccaacagaagaacatccattaacacatgtgaaagcacctgttattttaggtcatgagtttagtggtgaggtaatagagattggtgaaggagttacatctcataaagtgggagaccgcgttgttgtagagccaatttattcttgtggtaaatgtgaagcttgtaaacatggacattacaatgtttgtgaacaacttgttttccacggtcttggcggagaaggcggcggtttctctgaatatacagtagtaccagaagatatggttcatcacattccagatgaaatgacgtatgaacaaggtgcgcttgtagaaccagcagcagtagcagttcatgcagtacgtcaaagtaaattaaaagaaggggaagctgtagcggtatttggttgcggtccaattggacttcttgttatccaagcagctaaagcagcaggagcaactcctgttattgcagttgaactttctaaagaacgtcaagagttagcgaaattagcaggtgcggattatgtattaaatccagcaactcaagatgtgttagctgaaattcgtaacttaacaaatggtttaggtgtaaatgttagctttgaagtaacaggtgttgaagttgtactacgccaagcgattgaaagtacaagcttcgaaggacaaactgtaattgttagtgtatgggaaaaagacgcaacaattactccaaataacttagtattaaaagaaaaagaagttattggtattttaggataccgtcacatcttcccagctgttattaaattgattagctccggtcaaattcaagcagagaaattaattacgaaaaaaattacagtggatcaagttgttgaagaaggatttgaagcacttgtaaaagataaaacacaagtgaaaattcttgtttcacctaaataa

SEQ ID NO: 85 - protein, 350 aa, Bacillus cereus
MKALLWHNQRDVRVEEVPEPTVKPGTVKIKVKWCGICGTDLHEYLAGPIFIPTEEHPLTHVKAPVILGHEFSGEVIEIGEGVTSHKVGDRVVVEPIYSCGKCEACKHGHYNVCEQLVFHGLGGEGGGFSEYTVVPEDMVHHIPDEMTYEQGALVEPAAVAVHAVRQSKLKEGEAVAVFGCGPIGLLVIQAAKAAGATPVIAVELSKERQELAKLAGADYVLNPATQDVLAEIRNLTNGLGVNVSFEVTGVEVVLRQAIESTSFEGQTVIVSVWEKDATITPNNLVLKEKEVIGILGYRHIFPAVIKLISSGQIQAEKLITKKITVDQVVEEGFEALVKDKTQVKILVSPK

SEQ ID NO: 86 - DNA, 1053 bp, Bacillus cereus; identical to SEQ ID NO: 84

SEQ ID NO: 87 - protein, 350 aa, Bacillus cereus; identical to SEQ ID NO: 85

SEQ ID NO: 88 - DNA, 1113 bp, Lactococcus lactis
ttgcctgaaacgacaaccatcctatatagaggaggcgtttttatgcgcgcagcacgtttttacgaccgcggggatatccgcattgatgaaattaatgaaccaatagtaaaagctggccaagttggcattgatgtggcttggtgtggaatttgtggaacagatctccatgaatttttagatggcccaattttttgtccgtcagcagaacatcctaatccaattactggagaagtaccaccagtcactcttggacatgaaatgtctggggttgtaaattttataggtgaaggagtaagcggacttaaagtaggtgaccatgtcgttgtcgaaccttatatcgttcccgaagggactgatacaagtgaaactggacattataacctctcagaaggctcaaactttattggtttgggcggaaatggtggaggtttggctgaaaaaatttctgttgatgaacgttgggttcacaaaattcctgataacttaccattggatgaagctgctctaattgagccactatcagtcggctatcacgctgttgaacgagcaaatttaagtgaaaagagtacggtattagttgttggtgctggaccaattggactattaactgctgccgttgcaaaagcgcaaggacatactgttatcatcagtgaacctagtggacttcgtcgtaaaaaagcacaagaagcacaagttgctgattatttcttcaatccaattgaagatgacattcaagctaaagttcatgaaattaatgaaaaaggagtggacgcagcctttgaatgtacctctgtccaaccgggatttgacgcttgtctagatgcgattcgtatgggtggaacagttgtcattgtcgcaatttggggcaagcctgctagtgttgatatggcaaaattagtaatcaaagaagctaaccttttaggaacgattgcttataataacactcatccaaaaacaattgatttagtatcaacaggtaaaataaaattggaccaattcatcacagctaaaatcggtttggatgatttgattgataaaggattcgatacgctgattcatcataatgaaacagctgttaaaattttagtttcaccaactggtaaaggtctataa

SEQ ID NO: 89 - protein, 370 aa, Lactococcus lactis
MPETTTILYRGGVFMRAARFYDRGDIRIDEINEPIVKAGQVGIDVAWCGICGTDLHEFLDGPIFCPSAEHPNPITGEVPPVTLGHEMSGVVNFIGEGVSGLKVGDHVVVEPYIVPEGTDTSETGHYNLSEGSNFIGLGGNGGGLAEKISVDERWVHKIPDNLPLDEAALIEPLSVGYHAVERANLSEKSTVLVVGAGPIGLLTAAVAKAQGHTVIISEPSGLRRKKAQEAQVADYFFNPIEDDIQAKVHEINEKGVDAAFECTSVQPGFDACLDAIRMGGTVVIVAIWGKPASVDMAKLVIKEANLLGTIAYNNTHPKTIDLVSTGKIKLDQFITAKIGLDDLIDKGFDTLIHHNETAVKILVSPTGKGL

SEQ ID NO: 90 - DNA, 705 bp, Pyrococcus furiosus
atgaaggttgccgtaattactggggcatcccgtggaatcggggaagctatagcaaaggcccttgctgaagatggatattcccttgccttaggggctagaagtgttgataggttagagaagattgccaaggaactcagcgaaaaacatggggtggaggtattttacgactacctcgatgtatcaaaaccagaaagcgttgaagagtttgcaaggaaaacgctagctcactttggagatgtggacgttgttgtggccaatgcggggcttggttactttggtaggcttgaagagcttacagaagagcagttccacgaaatgattgaagtaaaccttttgggagtttggagaacaataaaagctttcttaaactccttaaagcggactggaggagtggctattgttgttacttcagatgtttctgcaaggctacttccatacggtggaggttatgtggcaactaaatgggctgcaagagcattggtaaggaccttccagattgagaatccagatgtgaggttcttcgagctaagacctggagcagtagatacatattttggagggagcaaagctgggaagccaaaggagcaagggtatttaaaacctgaggaagttgctgaggcagtaaaatacctcctaagacttccaaaggatgttagggttgaggaattaatgttgcgctcaatttatcaaaaacctgagtattga

SEQ ID NO: 91 - protein, 234 aa, Pyrococcus furiosus
MKVAVITGASRGIGEAIAKALAEDGYSLALGARSVDRLEKIAKELSEKHGVEVFYDYLDVSKPESVEEFARKTLAHFGDVDVVVANAGLGYFGRLEELTEEQFHEMIEVNLLGVWRTIKAFLNSLKRTGGVAIVVTSDVSARLLPYGGGYVATKWAARALVRTFQIENPDVRFFELRPGAVDTYFGGSKAGKPKEQGYLKPEEVAEAVKYLLRLPKDVRVEELMLRSIYQKPEY

SEQ ID NO: 92 - DNA, 1665 bp, Salmonella typhimurium
atgagatcgaaaagatttgaagcactggcgaaacgccctgtgaatcaggacggctttgttaaggagtggatcgaagaaggctttatcgcgatggaaagcccgaacgacccaaaaccgtcgataaaaatcgttaacggcgcggtaaccgagctggacggaaaaccggttagcgaattcgacctgatcgaccactttatcgcccgctacggcatcaacctgaaccgcgccgaagaagtgatggcgatggattcggtcaagctggctaacatgctgtgcgatccgaacgtcaagcgcagcgaaatcgttccgctaaccaccgcgatgaccccagcgaaaattgtcgaagtggtttcgcatatgaacgtggttgagatgatgatggcgatgcagaaaatgcgcgcccgccgtactccatctcaacaggcgcacgtcaccaacgttaaagacaacccggtgcaaattgccgccgatgccgccgaaggcgcatggcgcgggtttgacgaacaagagacgacggttgcggtagcgcgctatgcgccgttcaacgccatcgcgctgctggttggttctcaggtaggtcgtccgggggtactgactcaatgctcgctggaagaagccaccgagctgaagctcggcatgctgggccacacctgctacgccgaaaccatctccgtttacggcaccgagccggtcttcaccgacggtgacgataccccatggtcgaagggcttcttagcctcttcctacgcctctcgcggcctgaaaatgcgcttcacctccggctccggctccgaagtgcagatgggctacgccgaaggcaaatccatgctgtatctggaagcgcgctgcatctatatcaccaaagccgcgggcgttcaggggctgcaaaacggctccgtaagcagcatcggcgtaccgtctgccgtgccgtcaggcattcgtgccgtgctggcggaaaacctgatctgctcttcgctggatctggaatgcgcctccagtaacgaccagaccttcacccactccgatatgcgtcgtaccgctcgcctgctgatgcagttcctgccgggtaccgactttatctcctccggttattccgcggtgccgaactacgacaacatgttcgccggttccaacgaagatgcggaagactttgacgactacaacgttatccagcgtgacctgaaagtggacggcggtctgcgcccggttcgcgaagaggacgttatcgccatccgtaacaaagccgcccgcgcgctgcaggccgtgtttgccggaatgggactgccgccgattaccgatgaagaagttgaagccgcgacctatgcccacggttcgaaagatatgccggagcgcaacatcgtcgaagacatcaagttcgcccaggaaatcatcaataaaaaccgcaacggtctggaagttgtgaaagcgctggctcagggcgggtttaccgacgtggcccaggacatgctcaacatccagaaagccaagctaaccggcgactatttgcacacctccgccattatcgtcggcgacggacaagtgctctctgcggttaatgacgtcaatgactatgccggtccggcaacaggttatcgcctgcagggagaacgctgggaagagattaaaaacatccctggcgctcttgatcccaacgagattgattaa

SEQ ID NO: 93 - protein, 554 aa, Salmonella typhimurium
MRSKRFEALAKRPVNQDGFVKEWIEEGFIAMESPNDPKPSIKIVNGAVTELDGKPVSEFDLIDHFIARYGINLNRAEEVMAMDSVKLANMLCDPNVKRSEIVPLTTAMTPAKIVEVVSHMNVVEMMMAMQKMRARRTPSQQAHVTNVKDNPVQIAADAAEGAWRGFDEQETTVAVARYAPFNAIALLVGSQVGRPGVLTQCSLEEATELKLGMLGHTCYAETISVYGTEPVFTDGDDTPWSKGFLASSYASRGLKMRFTSGSGSEVQMGYAEGKSMLYLEARCIYITKAAGVQGLQNGSVSSIGVPSAVPSGIRAVLAENLICSSLDLECASSNDQTFTHSDMRRTARLLMQFLPGTDFISSGYSAVPNYDNMFAGSNEDAEDFDDYNVIQRDLKVDGGLRPVREEDVIAIRNKAARALQAVFAGMGLPPITDEEVEAATYAHGSKDMPERNIVEDIKFAQEIINKNRNGLEVVKALAQGGFTDVAQDMLNIQKAKLTGDYLHTSAIIVGDGQVLSAVNDVNDYAGPATGYRLQGERWEEIKNIPGALDPNEID

SEQ ID NO: 94 - DNA, 675 bp, Salmonella typhimurium
atggaaattaatgaaaaattgctgcgccagataattgaagacgtactccgcgatatgaagggcagcgataaacccgtctcgtttaatgcgcctgcggcatccacagcaccacagaccgctgcgcctgcgggcgacggctttctgaccgaagtgggcgaagcgcgccagggcactcagcaggacgaagtcattatcgccgtcggcccggcatttggcctggcgcaaaccgtcaatatcgtcggcttaccgcataagagcattctgcgcgaagtcattgccggtattgaagaagaaggcatcaaggcgcgcgtgattcgctgctttaaatcttccgacgtggcgttcgtcgccgttgaaggtaaccgcctgagcggatccggcatctccatcggcatccagtcgaaaggtactacggttatccaccagcaggggctaccgccgctctccaacctggagctgttcccgcaggcaccgctgctgacgctggaaacctaccgtcagattggtaaaaacgccgcccgctatgcgaaacgagaatcaccgcagccggtccctacgctcaatgaccagatggcacgcccgaagtaccaggcaaagtcggccattttgcatattaaagagaccaagtacgtcgtgacgggcaaaaacccgcaggaactgcgcgtggcgctttga

SEQ ID NO: 95 - protein, 224 aa, Salmonella typhimurium
MEINEKLLRQIIEDVLRDMKGSDKPVSFNAPAASTAPQTAAPAGDGFLTEVGEARQGTQQDEVIIAVGPAFGLAQTVNIVGLPHKSILREVIAGIEEEGIKARVIRCFKSSDVAFVAVEGNRLSGSGISIGIQSKGTTVIHQQGLPPLSNLELFPQAPLLTLETYRQIGKNAARYAKRESPQPVPTLNDQMARPKYQAKSAILHIKETKYVVTGKNPQELRVAL

SEQ ID NO: 96 - DNA, 522 bp, Salmonella typhimurium
atgaataccgacgcaattgaatcgatggtccgggacgtattgagccgcatgaacagcctgcagggcgatgcgccagcagcggctcctgcggcaggcggcacgtcccgcagcgcaaaggtcagcgactacccgctggcgaacaaacacccggaatgggtgaaaaccgccaccaataaaacgctggacgactttacgctggaaaacgtgctgagcaataaagtcaccgctcaggatatgcgtattaccccggaaaccctgcgcttacaggcctctatcgccaaagatgcgggtcgcgaccggctggcgatgaacttcgaacgcgccgccgaactgaccgcggtaccggacgatcgcattcttgaaatctacaacgcccttcgtccgtatcgttcaacgaaagaagagctgctcgctatcgccgacgatctcgaaaaccgttatcaggcaaagatttgcgcagctttcgttcgtgaagcggcagggctgtacgttgagcgtaaaaaactcaaaggcgacgattaa

SEQ ID NO: 97 - protein, 173 aa, Salmonella typhimurium
MNTDAIESMVRDVLSRMNSLQGDAPAAAPAAGGTSRSAKVSDYPLANKHPEWVKTATNKTLDDFTLENVLSNKVTAQDMRITPETLRLQASIAKDAGRDRLAMNFERAAELTAVPDDRILEIYNALRPYRSTKEELLAIADDLENRYQAKICAAFVREAAGLYVERKKLKGDD

SEQ ID NO: 98 - DNA, 1677 bp, Lactobacillus collinoides
ttggaacgtcaaaaaagatttgaaaaattagagaaacgtccagtgcatttagatgggttcgttaagaactgggacgacgaaggtttagttgcccttaacggtaagaacgatccaaagccaagcattacgatcgaaaacggtgttgttactgaaatggatggtaagaagaaggcagacttcgaccttatcgacaagtacatcgctgaatacgggatcaacttggacaatgctgaaaagactttaaacacagattcagttaagatcgccaacatgatgtgtgatcctaacgtctcccgtgctgaaattattgaatatacaactgctatgacaccagccaaggctgctgaagttatcagccagttaaacttcgctgaaatgatcatggcaactcaaaagatgcggccacgtcggacccctatgactcaagtccacgctaccaacactttggataacccagttgaaatcgctgctgatgctgccgaagctgcattacgtggggttcctgaagaagaaaccaccactgccattgctcggtatgcgccaatgaacgctatttcaatcatggttggggcccaagcaggccgtcctggtgttatcacccaatgttcagttgaagaagctgacgaattgagtttggggatgcgtgggtttactgcctatgctgaaaccatttcagtttatgggactgaccgggtcttcactgatggtgatgataccccttggtcaaaaggtttcttagcttcttgctacgcttcacgtggtttgaagatgcggtttacttcaggtgccggttcagaagctatgatgggctacactgaaggtaaatcaatgctttaccttgaagctcgttgtatctacattaccaaggcgtcaggtgttcaaggtctgcaaaacggtggtgttagttgtatcgggatgccaggtgccgtcgttggtggtatccgtgaagtcttaggtgaaaacttactatgtatgtcacttgatgttgaatgtgcttctggttgtgaccaagccttctctcactctgacattcgtcggactggccggatgattggccaattcatcgctggtactgattacctgtcatcaggttacgctgccgaagaaaacatggataacaccttcgctggttcaaacatggatgttctggactacgatgattacatcactttggaacgtgatatggctattaacggtggtatcatgccaattaccgaagaggaatctattaagattcgtcacaaggctgcggttgctatccaagctgtctttgatggcttaggcctaccacagatcactgatgaagaagttgaagccgcaacttatggcagcaattcaaacgacatgccaaaacgtgacatggttcaagatatgaaagctgctcaaggtctgatgactcgtggcattactgttgttgacgttatcaaggccttatatgaccatgatattaaagacgtcgctgaggctgtgcttaagttagcgcaacaaaaggtttgtggtgattacctgcaaacatctgctgtcttcttggatggttggaagtgtacttcagctattaacaacgctaacgattacaaaggcccaggtactggttaccgtctatgggaagacaaagacaaatgggatcgtctagaaaacgttccgtgggctttggatcctcagaagttggaattctaa

SEQ ID NO: 99 - protein, 558 aa, Lactobacillus collinoides
MERQKRFEKLEKRPVHLDGFVKNWDDEGLVALNGKNDPKPSITIENGVVTEMDGKKKADFDLIDKYIAEYGINLDNAEKTLNTDSVKIANMMCDPNVSRAEIIEYTTAMTPAKAAEVISQLNFAEMIMATQKMRPRRTPMTQVHATNTLDNPVEIAADAAEAALRGVPEEETTTAIARYAPMNAISIMVGAQAGRPGVITQCSVEEADELSLGMRGFTAYAETISVYGTDRVFTDGDDTPWSKGFLASCYASRGLKMRFTSGAGSEAMMGYTEGKSMLYLEARCIYITKASGVQGLQNGGVSCIGMPGAVVGGIREVLGENLLCMSLDVECASGCDQAFSHSDIRRTGRMIGQFIAGTDYLSSGYAAEENMDNTFAGSNMDVLDYDDYITLERDMAINGGIMPITEEESIKIRHKAAVAIQAVFDGLGLPQITDEEVEAATYGSNSNDMPKRDMVQDMKAAQGLMTRGITVVDVIKALYDHDIKDVAEAVLKLAQQKVCGDYLQTSAVFLDGWKCTSAINNANDYKGPGTGYRLWEDKDKWDRLENVPWALDPQKLEF

SEQ ID NO: 100 - DNA, 693 bp, Lactobacillus collinoides
gtgagttcagaaatcgatgaaacattgcttagaaatatcattaaaggcgttttaaatgaagttcaaaactctgatacgccaatttcctttggtggccaagatgcagccccagttgccggtgccaaggaaggtgccgcaccagaaaagaagttggattggttccaacacgttggaatcgccaaaccaggtttgtcaaaggatgaagttgtaattggtgttgccccagcatttgctgaagtgttgacgcaaactatgacgaagatccaacacaaagacatcctgcgtcaaatcattgccggagttgaagaagaaggtctcaaggcccgtgtcgttaaggtttatcggacttcagacgtttccttcgtttccgctgatgttgacaagttgtcaggttcaggaatttcagttgccgttcaatcaaaggggacaacgattattcaccaaaaggatcaagcaccgttgtcaaaccttgaattgttcccacaggctccagttttgacattggacgcttaccgtcaaatcggtaagaacgctgcccagtatgctaagggtatgtcaccaaccccagtgccaacaattaacgaccagatggcacgtgtgcaatatcaagcactttctgctttgatgcacatcaaggaaacaaaacaggttgttgttgggaagcctgctgaagaaattaaggtaaccttttag

SEQ ID NO: 101 - protein, 230 aa, Lactobacillus collinoides
MSSEIDETLLRNIIKGVLNEVQNSDTPISFGGQDAAPVAGAKEGAAPEKKLDWFQHVGIAKPGLSKDEVVIGVAPAFAEVLTQTMTKIQHKDILRQIIAGVEEEGLKARVVKVYRTSDVSFVSADVDKLSGSGISVAVQSKGTTIIHQKDQAPLSNLELFPQAPVLTLDAYRQIGKNAAQYAKGMSPTPVPTINDQMARVQYQALSALMHIKETKQVVVGKPAEEIKVTF

SEQ ID NO: 102 - DNA, 522 bp, Lactobacillus collinoides
atgagtgaagtagatgacttagtagctagaattgctgctcagctacaacaaagtggaaacgcttctagtgcctcaactagtgccggtacttctgctggttccgagaaagaattaggcgcagcagattacccactatttgaaaagcacccagatcaaatcaagacgccatcaggtaaaaatgttgaagaaatcaccttggaaaatgttattaacggcaaggtagacgcaaaggatatgcggattacgcccgcaaccctgaagttacaaggtgaaattgctgccaacgcaggtcggccagcaatccaacggaacttccagcgggcttctgaattaacttcagttcccgatgatgttgttttggacttatataattcattacggccattccgttcaaccaagcaagaattattggataccgccaaggagcttcgtgacaagtatcacgcacctatctgtgccggctggttcgaagaagcagccgaaaactacgaagtcaacaagaagttgaagggcgataactag

SEQ ID NO: 103 - protein, 173 aa, Lactobacillus collinoides
MSEVDDLVARIAAQLQQSGNASSASTSAGTSAGSEKELGAADYPLFEKHPDQIKTPSGKNVEEITLENVINGKVDAKDMRITPATLKLQGEIAANAGRPAIQRNFQRASELTSVPDDVVLDLYNSLRPFRSTKQELLDTAKELRDKYHAPICAGWFEEAAENYEVNKKLKGDN

SEQ ID NO: 104 - DNA, 1665 bp, Klebsiella pneumoniae
atgagatcgaaaagatttgaagcactggcgaaacgccctgtgaatcaggatggtttcgttaaggagtggattgaagagggctttatcgcgatggaaagtcctaacgatcccaaaccttctatccgcatcgtcaacggcgcggtgaccgaactcgacggtaaaccggttgacgagttcgacctgattgaccactttatcgcgcgctacggcattaatctcgcccgggccgaagaagtgatggccatggattcggttaagctcgccaacatgctctgcgacccgaacgttaaacgcagcgacatcgtgccgctcactaccgcgatgaccccggcgaaaatcgtggaagtggtgtcgcatatgaacgtggtcgagatgatgatggcgatgcaaaaaatgcgcgcccgccgcacgccgtcccagcaggcgcatgtcactaatatcaaagataatccggtacagattgccgccgacgccgctgaaggcgcatggcgcggctttgacgaacaggagaccaccgtcgccgtggcgcgctacgcgcggttcaacgccatcgccctgctggtgggttcacaggttggccgccccggcgtcctcacccagtgttcgctggaagaagccaccgagctgaaactgggcatgctgggccacacctgctatgccgaaaccatttcggtatacggtacggaaccggtgtttaccgatggcgatgacactccatggtcgaaaggcttcctcgcctcctcctacgcctcgcgcggcctgaaaatgcgctttacctccggttccggttctgaagtacagatgggctatgccgaaggcaaatcgatgctttatctcgaagcgcgctgcatctacatcaccaaagccgccggggtgcaaggcctgcagaatggctccgtcagctgtatcggcgtaccgtccgccgtgccgtccgggatccgcgccgtactggcggaaaacctgatctgctcagcgctggatctggagtgcgcctccagcaacgatcaaacctttacccactcggatatgcggcgtaccgcgcgtctgctgatgcagttcctgccaggcaccgacttcatctcctccggttactcggcggtgcccaactacgacaacatgttcgccggttccaacgaagatgccgaagacttcgatgactacaacgtgatccagcgcgacctgaaggtcgatgggggtctgcggccggtgcgtgaagaggacgtgatcgccattcgcaacaaagccgcccgcgcgctgcaggcggtatttgccggcatgggtttgccgcctattacggatgaagaggtagaagccgccacctacgcccacggttcaaaagatatgcctgagcgcaatatcgtcgaggacatcaagtttgctcaggagatcatcaacaagaaccgcaacggcctggaggtggtgaaagccctggcgaaaggcggcttccccgatgtcgcccaggacatgctcaatattcagaaagccaagctcaccggcgactacctgcatacctccgccatcattgttggcgagggccaggtgctctcggccgtgaatgacgtgaacgattatgccggtccggcaacaggctaccgcctgcaaggcgagcgctgggaagagattaaaaatatcccgggcgcgctcgatcccaatgaacttggctaa

SEQ ID NO: 105 - protein, 554 aa, Klebsiella pneumoniae
MRSKRFEALAKRPVNQDGFVKEWIEEGFIAMESPNDPKPSIRIVNGAVTELDGKPVDEFDLIDHFIARYGINLARAEEVMAMDSVKLANMLCDPNVKRSDIVPLTTAMTPAKIVEVVSHMNVVEMMMAMQKMRARRTPSQQAHVTNIKDNPVQIAADAAEGAWRGFDEQETTVAVARYARFNAIALLVGSQVGRPGVLTQCSLEEATELKLGMLGHTCYAETISVYGTEPVFTDGDDTPWSKGFLASSYASRGLKMRFTSGSGSEVQMGYAEGKSMLYLEARCIYITKAAGVQGLQNGSVSCIGVPSAVPSGIRAVLAENLICSALDLECASSNDQTFTHSDMRRTARLLMQFLPGTDFISSGYSAVPNYDNMFAGSNEDAEDFDDYNVIQRDLKVDGGLRPVREEDVIAIRNKAARALQAVFAGMGLPPITDEEVEAATYAHGSKDMPERNIVEDIKFAQEIINKNRNGLEVVKALAKGGFPDVAQDMLNIQKAKLTGDYLHTSAIIVGEGQVLSAVNDVNDYAGPATGYRLQGERWEEIKNIPGALDPNELG

SEQ ID NO: 106 - DNA, 687 bp, Klebsiella pneumoniae
atggaaattaacgaaacgctgctgcgccagattatcgaagaggtgctgtcggagatgaaatcaggcgcagataagccggtctcctttagcgcgccggcgtctgtcgcctctgccgcgccggtcgccgttgcgcctgtgtccggcgacagcttcctgacggaaatcggcgaagccaaacccggcacgcagcaggatgaagtcattattgccgtcgggccagcgtttggtctggcgcaaaccgccaatatcgtcggcattccgcataaaaatattctgcgcgaagtgatcgccggcattgaggaagaaggcatcaaagcccgggtgatccgctgctttaagtcatctgacgtcgccttcgtggcagtggaaggcaaccgcctgagcggctccggcatctcgatcggtattcagtcgaaaggcaccaccgtcatccaccagcgcggcctgccgccgctttccaatctggaactcttcccgcaggcgccgctgttaacgctggaaacctaccgtcagattggcaaaaacgccgcgcgctacgccaaacgcgagtcgccgcagccggtgccgacgcttaacgatcagatggctcgtcccaaataccaggcgaagtcggccattttgcacattaaagagaccaaatacgtggtgacgggcaaaaacccgcaggaactgcgcgtggcgctttaa

SEQ ID NO: 107 - protein, 228 aa, Klebsiella pneumoniae
MEINETLLRQIIEEVLSEMKSGADKPVSFSAPASVASAAPVAVAPVSGDSFLTEIGEAKPGTQQDEVIIAVGPAFGLAQTANIVGIPHKNILREVIAGIEEEGIKARVIRCFKSSDVAFVAVEGNRLSGSGISIGIQSKGTTVIHQRGLPPLSNLELFPQAPLLTLETYRQIGKNAARYAKRESPQPVPTLNDQMARPKYQAKSAILHIKETKYVVTGKNPQELRVAL

SEQ ID NO: 108 - DNA, 525 bp, Klebsiella pneumoniae
atgaataccgacgcaattgaatccatggtacgcgacgtgctgagccggatgaacagcctacaggacggggtaacgcccgcgccagccgcgccgacaaacgacaccgttcgccagccaaaagttagcgactacccgttagcgacctgccatccggagtgggtcaaaaccgctaccaataaaacgctcgatgacctgacgctggagaacgtattaagcgatcgcgttacggcgcaggacatgcgcatcactccggaaacgctgcgtatgcaggcggcgatcgcccaggatgccggacgcgatcggctggcgatgaactttgagcgggccgcagagctcaccgcggttcccgacgaccgaatccttgagatctacaacgccctgcgcccataccgttccacccaggcggagctactggcgatcgctgatgacctcgagcatcgctaccaggcacgactctgtgccgcctttgttcgggaagcggccgggctgtacatcgagcgtaagaagctgaaaggcgacgattaa

SEQ ID NO: 109 - protein, 174 aa, Klebsiella pneumoniae
MNTDAIESMVRDVLSRMNSLQDGVTPAPAAPTNDTVRQPKVSDYPLATCHPEWVKTATNKTLDDLTLENVLSDRVTAQDMRITPETLRMQAAIAQDAGRDRLAMNFERAAELTAVPDDRILEIYNALRPYRSTQAELLAIADDLEHRYQARLCAAFVREAAGLYIERKKLKGDD

SEQ ID NO: 110 - DNA, 1833 bp, Klebsiella oxytoca
atgcgatatatagctggcattgatatcggcaactcatcgacggaagtcgccctggcgaccctggatgaggctggcgcgctgacgatcacccacagcgcgctggcggaaaccaccggaatcaaaggcacgttgcgtaacgtgttcgggattcaggaggcgctcgccctcgtcgccagaggcgccgggatcgccgtcagcgatatttcgctcatccgcatcaacgaagcgacgccggtgattggcgatgtggcgatggaaaccattaccgaaaccatcatcaccgaatcgaccatgatcggccataacccgaaaacgcccggcggcgcggggcttggcacaggcatcaccattacgccgcaggagctgctaacccgcccggcggacgcgccctatatcctggtggtgtcgtcggcgttcgattttgccgatatcgccagcgtgattaacgcttccctgcgcgccgggtatcagattaccggcgtcattttacagcgcgacgatggcgtgctggtcagcaaccggctggaaaaaccgctgccgatcgttgacgaagtgctgtacatcgaccgcattccgctggggatgctggcggcgattgaggtcgccgttccggggaaggtcatcgaaaccctctctaacccttacggcatcgccaccgtctttaacctcagccccgaggagacgaagaacatcgtcccgatggcccgggcgctgattggcaaccgttccgccgtggtggtcaaaacgccatccggcgacgtcaaagcgcgcgcgatacccgccggtaatcttgagctgctggcccagggccgtagcgtgcgcgtggatgtggccgccggcgccgaagccatcatgaaagcggtcgacggctgcggcaggctcgataacgtcaccggcgaatccggcaccaatatcggcggcatgctggaacacgtgcgccagaccatggccgagctgaccaacaagccgagcagcgaaatatttattcaggacctgctggccgttgatacctcggtaccggtgagcgttaccggcggtctggccggggagttctcgctggagcaggccgtgggcatcgcctcgatggtgaaatcggatcgcctgcagatggcaatgatcgcccgcgaaatcgagcagaagctcaatatcgacgtgcagatcggcggcgcagaggccgaagccgccatcctgggggcgctgaccacgccgggcaccacccgaccgctggcgatcctcgacctcggcgcgggctccaccgatgcctccatcatcaaccccaaaggcgacatcatcgccacccatctcgccggcgcaggcgacatggtgacgatgattattgcccgcgagctggggctggaagaccgctatctggcggaagagatcaagaagtacccgctggctaaggtggaaagcctgttccatttacgccacgaggacggcagcgtgcagttcttctccacgccgctgccgcccgccgtgttcgcccgcgtctgcgtggtgaaagcggacgaactggtgccgctgcccggcgatttagcgctggaaaaagtgcgcgccattcgccgcagcgccaaagagcgggtctttgtcaccaacgccctgcgcgcgctgcgtcaggtcagccccaccggcaacattcgcgatattccgttcgtggtgctggtcggcggttcgtcgctggatttcgaagtcccgcagctggtcaccgatgcgctggcgcactaccgcctggttgccggacggggaaatattcgcggcagcgagggcccccgaaacgcggtggccaccggcctgattctctcctggcataaggagtttgcgcatgaacggtaa

SEQ ID NO: 111 - protein, 610 aa, Klebsiella oxytoca
MRYIAGIDIGNSSTEVALATLDEAGALTITHSALAETTGIKGTLRNVFGIQEALALVARGAGIAVSDISLIRINEATPVIGDVAMETITETIITESTMIGHNPKTPGGAGLGTGITITPQELLTRPADAPYILVVSSAFDFADIASVINASLRAGYQITGVILQRDDGVLVSNRLEKPLPIVDEVLYIDRIPLGMLAAIEVAVPGKVIETLSNPYGIATVFNLSPEETKNIVPMARALIGNRSAVVVKTPSGDVKARAIPAGNLELLAQGRSVRVDVAAGAEAIMKAVDGCGRLDNVTGESGTNIGGMLEHVRQTMAELTNKPSSEIFIQDLLAVDTSVPVSVTGGLAGEFSLEQAVGIASMVKSDRLQMAMIAREIEQKLNIDVQIGGAEAEAAILGALTTPGTTRPLAILDLGAGSTDASIINPKGDIIATHLAGAGDMVTMIIARELGLEDRYLAEEIKKYPLAKVESLFHLRHEDGSVQFFSTPLPPAVFARVCVVKADELVPLPGDLALEKVRAIRRSAKERVFVTNALRALRQVSPTGNIRDIPFVVLVGGSSLDFEVPQLVTDALAHYRLVAGRGNIRGSEGPRNAVATGLILSWHKEFAHER

SEQ ID NO: 112 - DNA, 378 bp, Klebsiella oxytoca
atgaacggtaatcacagcgccccggccatcgcgatcgccgtcatcgacggctgcgacggcctgtggcgcgaagtgctgctgggtatcgaagaggaaggtatccctttccggctccagcatcacccggccggagaggtcgtggacagcgcctggcaggcggcgcgcagctcgccgctgctggtgggcatcgcctgcgaccgccatatgctggtcgtgcactacaagaatttacccgcatcggcgccgctttttacgctgatgcatcatcaggacagtcaggcccatcgcaacaccggtaataacgcggcacggctggtcaaggggatccctttccgggatctgaatagcgaagcaacaggagaacagcaggatgaataa

SEQ ID NO: 113 - protein, 125 aa, Klebsiella oxytoca
MNGNHSAPAIAIAVIDGCDGLWREVLLGIEEEGIPFRLQHHPAGEVVDSAWQAARSSPLLVGIACDRHMLVVHYKNLPASAPLFTLMHHQDSQAHRNTGNNAARLVKGIPFRDLNSEATGEQQDE

SEQ ID NO: 114 - DNA, 1833 bp, Salmonella typhimurium
atgcgatatatagctggcattgacatcggtaactcatcaacggaagtcgcactggcgcggcaagatgagactggcgcactgacgattacacacagcgcgctggcggaaaccaccgggatcaaaggcacgttgcgtaacgtgttcggcattcaggaagcgctcgccctcgtcgcaaagcgcgcggggatcaatgtcagagatatttcgctcatccgcattaacgaagccacgccggtgattggcgatgtggcgatggaaaccattaccgaaaccatcatcaccgaatcgacaatgatcggccataacccaaaaacgccgggcggagcaggccttggtgtgggtatcacgattacgccggaggagctgttaacccgcccggcggactcgtcctatattctggtggtatcgtcagcctttgattttgctgatatcgccaatgttatcaacgcctcaatgcgcgccggataccagattaccggcgtcattttgcagcgcgacgatggcgtactggtcagcaaccggctggaaaaatcgctaccgattgtcgatgaagttctgtacatcgaccgcattccgctggggatgctggcggcgattgaagtcgccgtgccgggaaaggttatcgaaaccctctctaacccttacggcatcgccaccgtatttaatctcaacgccgatgagacaaaaaacatcgtcccgatggcgcgcgcgctgattggcaaccgttccgccgtggtggttaaaacgccatccggcgacgtcaaagcgcgcgcaatacccgccggtaacctggagctgcaggctcagggtcgtaccgtgcgcgtggatgttgccgccggtgccgaagccatcatgaaagcggtggacggttgcggcaagctcgacaacgtcaccggcgaggccgggaccaatatcggcggcatgctggagcacgtgcgccagaccatggccgaactgaccaacaagccgagcagtgagattttcattcaggatctactggccgttgacacctcggttccggtgagcgtcaccggcggtctggccggggagttctcgctggagcaggccgtcggcatcgcctcgatggtgaaatcagaccgtctgcagatggcgatgattgcccgtgaaattgagcagaagcttaatatcgacgtgcagatcggcggcgctgaggctgaagccgccattctgggcgcgctgaccacgccgggtaccacccgaccgctggcgatcctcgacctcggcgcgggctccaccgatgcctccatcatcaaccctaaaggcgaaatcatcgccacccatctcgccggggcaggcgacatggtcacgatgattattgcccgcgaactggggctggaagaccgctatctggcggaagagatcaaaaaatacccgctggctaaggtcgaaagcctgttccacttacgccacgaggacggcagcgtccagttcttcccgacgccgctgcctcccgccgtgttcgcccgcgtctgcgtggtgaaaccggacgaactggtgccgcttcccggcgacttagcgctggaaaaagtgcgcgccattcgccgcagcgctaaagaacgcgtctttgtcaccaacgccctgcgcgcgctgcgccaggtcagtccaaccggcaacattcgcgatattccgttcgtggtgctggtcggcggctcgtcgctggatttcgaagttccgcagctggtcaccgatgcgctggcgcactaccgcctggtcgccgggcgaggaaatattcgcggcagcgaaggcccaagaaacgcggtggccaccggcctgattctctcctggcataaggagtttgcgcatggacagtaa

SEQ ID NO: 115 - protein, 610 aa, Salmonella typhimurium
MRYIAGIDIGNSSTEVALARQDETGALTITHSALAETTGIKGTLRNVFGIQEALALVAKRAGINVRDISLIRINEATPVIGDVAMETITETIITESTMIGHNPKTPGGAGLGVGITITPEELLTRPADSSYILVVSSAFDFADIANVINASMRAGYQITGVILQRDDGVLVSNRLEKSLPIVDEVLYIDRIPLGMLAAIEVAVPGKVIETLSNPYGIATVFNLNADETKNIVPMARALIGNRSAVVVKTPSGDVKARAIPAGNLELQAQGRTVRVDVAAGAEAIMKAVDGCGKLDNVTGEAGTNIGGMLEHVRQTMAELTNKPSSEIFIQDLLAVDTSVPVSVTGGLAGEFSLEQAVGIASMVKSDRLQMAMIAREIEQKLNIDVQIGGAEAEAAILGALTTPGTTRPLAILDLGAGSTDASIINPKGEIIATHLAGAGDMVTMIIARELGLEDRYLAEEIKKYPLAKVESLFHLRHEDGSVQFFPTPLPPAVFARVCVVKPDELVPLPGDLALEKVRAIRRSAKERVFVTNALRALRQVSPTGNIRDIPFVVLVGGSSLDFEVPQLVTDALAHYRLVAGRGNIRGSEGPRNAVATGLILSWHKEFAHGQ

SEQ ID NO: 116 - DNA, 372 bp, Salmonella typhimurium
atggacagtaatcacagcgccccggctatcgtcattaccgttatcaacgactgcgccagcctctggcacgaagtgctgctgggcattgaagaggaaggcatccctttcctgcttcagcatcacccggctggagatatcgttgacagcgcctggcaggcggcgcgcagctcgccgctgctggtcggcattgcctgcgatcgacactcgctggtcgtgcattacaagaatttacccgcatcggcgccgctttttacgctgatgcatcatcaggacagtcaggcccaacgcaacaccggtaataacgcggcacggctggtcaaagggatccctttcgggatctccatgcttaatcacaggagaacggcagtatga

SEQ ID NO: 117 - protein, 123 aa, Salmonella typhimurium
MDSNHSAPAIVITVINDCASLWHEVLLGIEEEGIPFLLQHHPAGDIVDSAWQAARSSPLLVGIACDRHSLVVHYKNLPASAPLFTLMHHQDSQAQRNTGNNAARLVKGIPFGISMLNHRRTAV

SEQ ID NO: 118 - DNA, 1833 bp, Lactobacillus collinoides
atgacacgtgtaattggtgttgatatcgggaattcctctacagaagttgcgcttgctgatgtgtctgacagtggtgaagtaaatttcattaattctggaatttccgatacaactggcattaaaggtactaaacaaaatttgatcggggtgcgtaaatccatccagatcgttttgaaaaagtcgaatatgcaaatttccgatgttgacctgattcggatcaacgaagcaacgcccgttatcggtgatgttgccatggagaccatcaccgaaacggtgattactgaatcgacgatgatcggccacaacccagggactcctgggggtgtcggtactggttctggttacacggtgaatttgcttgatttgttgagccaaacggataaggatcgtccttatatcgttatcatctcgaaagaaatcgattttgctgacgcagctaagctgatcaacgcttatgtggcttctggttataatattaccgctgccattctgcaaagtgatgatggggtgctgatcaataatcggttgacccataagattcccatcgtggatgaagtctcacagatcgacaaggtaccgttgaacatgcttgccgcagtggaagttgcaccgcctggcaaagtaattgctcaactttccaacccgtatggcattgccacactgttcgaactttcctctgaagaaaccaagaacattgtgccagttgcccgagccttaatcggaaaccggtcagcggttgttattaaaacccctgccggtgatgttaaagctcgtgttatcccagccgggaaaatcttgatcaatggccaaccgaatggtcatggtgaagttaacgttgcggctggtgccgatgccatcatgaaaaaggtgaacgagttcgatagtgtcgatgacattaccggtgaatcgggcactaacgttggtgggatgcttgaaaaagttcgtcaaacaatggctgagttgaccgacaagcaaaatagcgacattgccattcaagatttattagctgtcaatacgtccgttccagtaacggtgcgtggtggtctggctggtgaattctcaatggaacaagccgttgggattgctgctatggtcaaatctgatcacttgcaaatgcaagcgattgcagacctgatgaaagatgaatttcacgttcaagtcgaaatcggcggtgctgaagctgaatcagccatcctcggtgcgctaacaacgccagggacgacaaaaccaattgccatccttgatttgggggctggttcaacggatgcatcaattatcaaccaaaaggacgaaaaggtcgctattcacttggctggtgccggtgatatggttaccatgatcatcaattctgaacttgggttggaagacccatatttagctgaggatattaagaaatatccgctggctaaagttgataatctattccagctacggcatgaagatggtgccgttcaattctttgaagatccattacctgctgatttatttgccagagttgtggctgttaaaccagatggttacgaaccacttcctggtaatttgagtatcgagaaagttaaaatcgtccgtcaaactgctaagaagcgggtgttcgtaacgaacgcaattcgtgccttacaccacgttagcccaacaggtaatatccgagatatcccatttgtggtcattgtcggcggctcagccctcgattttgaaattccacaattggtcaccgatgaattatcacactttaacttagttgcaggtcgtggtaatattcggggaattgaaggtccacggaacgccgtggcaactggtttgattctttcatacgcgagtgagaagaggggatag

SEQ ID NO: 119 - protein, 610 aa, Lactobacillus collinoides
MTRVIGVDIGNSSTEVALADVSDSGEVNFINSGISDTTGIKGTKQNLIGVRKSIQIVLKKSNMQISDVDLIRINEATPVIGDVAMETITETVITESTMIGHNPGTPGGVGTGSGYTVNLLDLLSQTDKDRPYIVIISKEIDFADAAKLINAYVASGYNITAAILQSDDGVLINNRLTHKIPIVDEVSQIDKVPLNMLAAVEVAPPGKVIAQLSNPYGIATLFELSSEETKNIVPVARALIGNRSAVVIKTPAGDVKARVIPAGKILINGQPNGHGEVNVAAGADAIMKKVNEFDSVDDITGESGTNVGGMLEKVRQTMAELTDKQNSDIAIQDLLAVNTSVPVTVRGGLAGEFSMEQAVGIAAMVKSDHLQMQAIADLMKDEFHVQVEIGGAEAESAILGALTTPGTTKPIAILDLGAGSTDASIINQKDEKVAIHLAGAGDMVTMIINSELGLEDPYLAEDIKKYPLAKVDNLFQLRHEDGAVQFFEDPLPADLFARVVAVKPDGYEPLPGNLSIEKVKIVRQTAKKRVFVTNAIRALHHVSPTGNIRDIPFVVIVGGSALDFEIPQLVTDELSHFNLVAGRGNIRGIEGPRNAVATGLILSYASEKRG

SEQ ID NO: 120 - DNA, 351 bp, Lactobacillus collinoides
atggcatttgattctgaacgtccgtcaattctattggcgacaccaacgggttctaatggccaacttccagaagttctaaaaccaatgctcaatggtattgaagaagaacagattccttttcagattctcgatatggaaggcggttcagcagttgagcgggcttataacgcgtcagttgcttcacgattatcagtgggcgttgggtttgatgatgcacatatcattgtgcattataaaaacttgaaaccagaaaaaccgctgtttgatgttgccatcactgatgcagcatccattcgtaaagttggcgcaaacgccgctcgacttgtaaagggagttccattcaagaagtaa

SEQ ID NO: 121 - protein, 116 aa, Lactobacillus collinoides
MAFDSERPSILLATPTGSNGQLPEVLKPMLNGIEEEQIPFQILDMEGGSAVERAYNASVASRLSVGVGFDDAHIIVHYKNLKPEKPLFDVAITDAASIRKVGANAARLVKGVPFKK

SEQ ID NO: 122 - protein, 453 aa, Vibrio fluvialis
MNKPQSWEARAETYSLYGFTDMPSLHQRGTVVVTHGEGPYIVDVNGRRYLDANSGLWNMVAGFDHKGLIDAAKAQYERFPGYHAFFGRMSDQTVMLSEKLVEVSPFDSGRVFYTNSGSEANDTMVKMLWFLHAAEGKPQKRKILTRWNAYHGVTAVSASMTGKPYNSVFGLPLPGFVHLTCPHYWRYGEEGETEEQFVARLARELEETIQREGADTIAGFFAEPVMGAGGVIPPAKGYFQAILPILRKYDIPVISDEVICGFGRTGNTWGCVTYDFTPDAIISSKNLTAGFFPMGAVILGPELSKRLETAIEAIEEFPHGFTASGHPVGCAIALKAIDVVMNEGLAENVRRLAPRFEERLKHIAERPNIGEYRGIGFMWALEAVKDKASKTPFDGNLSVSERIANTCTDLGLICRPLGQSVVLCPPFILTEAQMDEMFDKLEKALDKVFAEVA

SEQ ID NO: 123 - DNA, 1122 bp, Erwinia caratovora subsp. atroseptica
atgtctgacggacgactcaccgcactttttcctgcattcccacacccggcgtccaatcagcccgtatttgccgaggcttcaccgcacgacgacgagttaatgacgcaggccgtaccgcaggtttcctgtcagcaggcgttggcgattgcgcagcaagaatatggcttgtctgggcagatgtcgctgcttcagggcgagcgtgatgtgaatttctgtctgacggtgacgccagatgaacgctacatgctgaaagtcatcaatgcggcagaacctgccgacgtcagcaatttccaaaccgcgctgctgctgcatcttgcccgtcaggcacctgaactgcccgtaccgcgtatcaggtcgacaaaagcgggtcagtcggaaacaggcgttgagatcgatggtgtactgctgcgtgtgcggcttgtgagctatctggcaggaatgccgcagtatctggcctcaccgtcaacggcgctgatgccgcagttggggggaacgctggcgcagttggataacgcgcttcacagctttacgcatccggcggcaaaccgtgcgctgctgtgggatatcagccgggcagagcaggtgcgtccttacctcgatttcgtttctgaaccgcagcagtatcagcatcttcagcgtatttttgaccgttatgacagtaacgttgctcctctgttgacgacgctacgtcgtcaggtcattcataacgatctgaatccgcataacgtgctggtggatggatcgtcgccgacgcgggttactggcattatcgattttggcgatgccgtatttgccccgttaatttgcgaagtcgcgacggcactggcgtatcagatcggcgatggaaccgatttgttggagcatgttgtgccgtttgttgcggcctatcaccaacgcattccgttagcaccggaggagattgcgctgttacccgatctgatagcgacccgtatggcgctgaccctgaccattgcgcagtggcgagcatcgcgttatcccgacaatcgggagtatctgctgcgtaacgtgccgcgctgttggcacagtttgcagcgcattgcgacctattcccatgcgcaatttttgactcgcctacagcaggtttgcccggagaatgcgcga

SEQ ID NO: 124 - protein, 374 aa, Erwinia caratovora subsp. atroseptica
MSDGRLTALFPAFPHPASNQPVFAEASPHDDELMTQAVPQVSCQQALAIAQQEYGLSGQMSLLQGERDVNFCLTVTPDERYMLKVINAAEPADVSNFQTALLLHLARQAPELPVPRIRSTKAGQSETGVEIDGVLLRVRLVSYLAGMPQYLASPSTALMPQLGGTLAQLDNALHSFTHPAANRALLWDISRAEQVRPYLDFVSEPQQYQHLQRIFDRYDSNVAPLLTTLRRQVIHNDLNPHNVLVDGSSPTRVTGIIDFGDAVFAPLICEVATALAYQIGDGTDLLEHVVPFVAAYHQRIPLAPEEIALLPDLIATRMALTLTIAQWRASRYPDNREYLLRNVPRCWHSLQRIATYSHAQFLTRLQQVCPENAR

SEQ ID NO: 125 - DNA, 1272 bp, Erwinia caratovora subsp. atroseptica
atgacagcgacagaagctttgctggcgcgccgtcagcgagtgttgggcggcggttatcgcctgttttatgaagagccgctgcatgtcgcgcgcggcgagggcgtgtggctgttcgatcaccaagggaaacgttatctggatgtctacaataatgtggcttcggtcggacattgccaccccgcggtggttgaagccgtggcgcgacagagcgcacaactcaatacccacacgcgctatttgcaccacgcgattgtcgattttgcggaagatttgctgagcgaatttcccgccgaattgaacaatgtaatgctgacctgtaccggcagtgaggctaacgatctggcgctgcgtatcgcccgacatgtcacgggcgggacggggatgttggtgacgcgctgggcgtatcacggcgtgaccagcgcgctggcggaactgtctccgtcgctgggggatggcgttgtgcgcggtagccatgtgaagctgatcgacgcgccagacacttatcgtcagcccggtgcatttcttaccagcattcgtgaagcgctggcgcagatgcaacgggaaggtattcgtcctgcggcgctgctggtagataccattttttccagcgatggcgtgttctgtgcgccggaaggcgaaatggcacaggcggcggcgttgatccgtcaggcgggcgggctgtttattgcggatgaagtgcagccgggcttcgggcgcaccggggaatcactgtggggctttgcgcgccacaatgtcgtccctgatttggtgagtctagggaaaccgatgggcaacggacatcccatcgctggattggtggggcgttccgctctgttcgacgcatttgggcgcgatgtgcgctatttcaatacctttggcggcaatccggtttcctgtcaggcggcgcacgcggtgctgcgggtgattcgggaagagcagttgcagcagaatgcccagcgggtcggtgattatctgcggcaagggttgcagcaactggcgcagcatttcccgctgattggtgatattcgggcttacggcctgtttattggtgcggagctggtcagcgatcgcgaaagtaaaacgccggcaagtgaatccgcgttgcaggtggtgaatgcgatgcgccaacgtggtgtgctcatcagcgcgacggggccagcggcgaacatactgaaaattcgcccgccgctggtgtttctggaagaacacgccgatgtgttcttaaccacgctgagtgacgttttagcgctcatcggcactcgtgcacagaga

SEQ ID NO: 126 - protein, 424 aa, Erwinia caratovora subsp. atroseptica
MTATEALLARRQRVLGGGYRLFYEEPLHVARGEGVWLFDHQGKRYLDVYNNVASVGHCHPAVVEAVARQSAQLNTHTRYLHHAIVDFAEDLLSEFPAELNNVMLTCTGSEANDLALRIARHVTGGTGMLVTRWAYHGVTSALAELSPSLGDGVVRGSHVKLIDAPDTYRQPGAFLTSIREALAQMQREGIRPAALLVDTIFSSDGVFCAPEGEMAQAAALIRQAGGLFIADEVQPGFGRTGESLWGFARHNVVPDLVSLGKPMGNGHPIAGLVGRSALFDAFGRDVRYFNTFGGNPVSCQAAHAVLRVIREEQLQQNAQRVGDYLRQGLQQLAQHFPLIGDIRAYGLFIGAELVSDRESKTPASESALQVVNAMRQRGVLISATGPAANILKIRPPLVFLEEHADVFLTTLSDVLALIGTRAQR

SEQ ID NO: 127 - DNA, 35 bp, Artificial Seqquence
  Primer
ctccggaattcatgtctgacggacgactcaccgca

SEQ ID NO: 128 - DNA, 46 bp, Artificial Seqquence
  Primer
ttccaatgcattggctgcagttatctctgtgcacgagtgccgatga

SEQ ID NO: 129 - DNA, 40 bp, Artificial Seqquence
  Primer
aacagccaagcttggctgcagtcatcgcgcattctccggg

SEQ ID NO: 130 - DNA, 40 bp, Artificial Seqquence
  Primer
tctccggaattcatgacgtctgaaatgacagcgacagaag

SEQ ID NO: 131 - DNA, 33 bp, Artificial Sequence
  Primer
gctaacaggaggaagaattcatggggggttctc

SEQ ID NO: 132 - DNA, 33 bp, Artificial Sequence
  Primer
gagaaccccccatgaattcttcctcctgttagc

SEQ ID NO: 133 - DNA, 723 bp, Klebsiella terrigena
atgcaaaaagtcgcacttgtcaccggcgccggtcagggcatcggtaaagctatcgccctgcgtctggtgaaggatggatttgccgtggcaatcgccgattacaacgacgctacggccacagcggtagccgctgaaatcaaccaggccggcggccgcgcggtggccattaaggtcgacgtctcgcgccgggaccaggttttcgccgccgttgagcaggcgcgtaaagccctgggcggattcaacgttatcgtcaacaacgccggcatcgcgccgtcaacgccgatcgagtccatcaccgaggagatcgtcgaccgggtctataacatcaacgttaagggcgtcatctgggggatgcaggcggcggtggaggccttcaaaaaagaggggcacggcgggaagatcgtcaacgcctgctcccaggccggccacgtcggcaacccggagctggcggtctacagttcgagtaaattcgccgtgcgcggcctgacgcaaaccgccgcccgcgatctggcgccgctgggcatcaccgttaacggcttctgcccagggatcgttaagacgccaatgtgggcggagattgaccgtcagtgtcggaagcggcgggcaaaccgctgggctacggcacggctgaatttgccaaacgcatcacccttggccgcctgtcggagcctgaagacgtcgccgcctgcgtgtcgttcctcgccagcccggattccgactata

SEQ ID NO: 134 - protein, 241 aa, Klebsiella terrigena
MQKVALVTGAGQGIGKAIALRLVKDGFAVAIADYNDATATAVAAEINQAGGRAVAIKVDVSRRDQVFAAVEQARKALGGFNVIVNNAGIAPSTPIESITEEIVDRVYNINVKGVIWGMQAAVEAFKKEGHGGKIVNACSQAGHVGNPELAVYSSSKFAVRGLTQTAARDLAPLGITVNGFCPGIVKTPMWAEIDRQCRKRRANRWATARLNLPNASPLAACRSLKTSPPACRSSPARIPTI

SEQ ID NO: 135 - protein, 554 aa, Clostridium pasteurianum
MKSKRFQVLSERPVNKDGFIGEWPEEGLIAMSSPNDPKPSIKIKEGKVIELDGKNREDFDMIDRFIANYGINLNRAEDVIKMDSVKLAKMLVDINVDRKTIVELTTAMTPAKIVEVVGNMNVVEMMMALQKMRARKTPSNQCHVTNLKDNPVQIAADAAEAAIRGFDEQETTVGIVRYAPFNALALLVGAQVGRGGVLTQCAIEEATELELGMRGLTSYAETVSVYGTENVFTDGDDTPWSKAFLASAYASRGLKMRFTSGSGSEALMGYAEGKSMLYLEARCIYITKAAGVQGLQNGSVSCIGMTGALPSGIRAVLGENLITTMLDIEVASANDQTFSHSDIRRTARMLMQMLPGTDFIFSGYSSVPNYDNMFAGSNFDAEDFDDYNVIQRDLMVDGGLRPVSEEEVITIRNKAARAIQAVFEGLKLPAITDEEVEAVTYSHGSKDVPERNVVEDLKAAEEMINRGITGIDVVKALSKHGFDDIAENILNMLKQRISGDYLQTSAIIDKNFNVVSAVNDCNDYMGPGTGYRLSKERWDEIKNIPNAMKPEDIK

SEQ ID NO: 136 - protein, 179 aa, Clostridium pasteurianum
MELKEKDIALSGNQSNEVVIGIAPAFGKYQHQSIVGVPHDKILRELIAGIEEEGLKSRVVRIIRTSDVSFIAHDAAVLSGSGIGIGIQSKGTTVIHQKDLLPLNNLELFPQAPLLDLDIFRLIGKNAAKYAKGESPNPVPTRNDQMVRPKFQAKAALLHIKETKHVVQNAKPIELEIIS

SEQ ID NO: 137 - protein, 146 aa, Clostridium pasteurianum
MSDITNNIKVDYENDYPLAAKRSEWIKTPTGKNLKDITLEAVIDENVKAEDVRISRDTLELQAQVAEGSGRCAIARNFRRAAELISISDERILEIYNALRPYRSTKNELLAIADELEEKYDAKVNADFIREAAEVYSKRNKVRIED

SEQ ID NO: 138 - protein, 555 aa, Escherichia blattae
MRRSKRFEVLEKRPVNQDGLIGEWPEEGLIAMGSPWDPPSSVKVEQGRIVELDGKARADFDMIDRFIADYAINIEETEHAMGLDALTIARMLVDINVSRAEIIKVTTAITPAKAVEVMSHMNVVEMMMALQKMRARRTPSNQCHVTNLKDNPVQIAADAAEAGIRGFSEQETTVGIARYAPFNALALLIGSQSGRPGVLTQCSVEEATELELGMRGFTSYAETVSVYGTEAVFTDGDDTPWSKAFLASAYASRGLKMRYTSGTGSEALMGYAESKSMLYLESRCIFITKGAGVQGLQNGAVSCIGMTGAVPSGIRAVLAENLIASMLDLEVASANDQTFSHSDIRRTARTLMQMLPGTDFIFSGYSAVPNYDNMFAGSNFDAEDFDDYNILQRDLMVDGGLRPVSEEETIAIRNKAARAVQAVFRELGLPPVTDEEVTAATYAHGSKDMPPRNVVEDLSAVEEMMKRNITGLDIVRALSVNGFDDVANNILNMLRQRVTGDYLQTSAILDREFEVVSAVNDINDYQGPGTGYRISPQRWEEIKNIATVIQPDSIE

SEQ ID NO: 139 - protein, 196 aa, Escherichia blattae
METTQKKAPVFTLNLVESGVAKPGERSDEVVIGVGPAFDKYQHKTLIDMPHKAIIKELVAGVEEEGLHARVVRILRTSDVSFMAWDAANLSGSGIGIGIQSKGTTVIHQRDLLPLSNLELFSQAPLLTLETYRQIGKNAARYARKESPSPVPVVNDQMVRPKFMAKAALFHIKETKHVVADAKPVTLNIEITREEA

SEQ ID NO: 140 - protein, 141 aa, Escherichia blattae
MTTTKMSAADYPLASRCPERIQTPTGKPLTDITLENVLAGKVGPQDVRISRETLEYQAQIAEQMHRHAIARNLRRAGELIAIPDARILEIYNALRPYRSSVEELLAIADELETRYQATVNAAFIREAAEVYRQRDKLRKEA

SEQ ID NO: 141 - protein, 555 aa, Citrobacter freundii
MRRSKRFEVLAQRPVNQDGLIGEWPEEGLIAMESPYDPASSVKVENGRIVELDGKSRAEFDMIDRFIADYAINVPEAERAMQLDALEIARMLVDIHVSREEIIAITTAITPAKRLEVMAQMNVVEMMMALQKMRARRTPSNQCHVTNLKDNPVQIAADAAEAGIRGFSEQETTVGIARYAPFNALALLVGSQCGAPGVLTQCSVEEATELELGMRGLTSYAETVSVYGTESVFTDGDDTPWSKAFLASAYASRGLKMRYTSGTGSEALMGYSESKSMLYLESRCIFITKGAGVQGLQNGAVSCIGMTGAVPSGIRAVLAENLIASMLDLEVASANDQTFSHSDIRRTARTLMQMLPGTDFIFSGYSAVPNYDNMFAGSNFDAEDFDDYNILQRDLMVDGGLRPVTEEETIAIRNKAARAIQAVFRELGLPLISDEEVDAATYAHGSKDMPARNVVEDLAAVEEMMKRNITGLDIVGALSSSGFEDIASNILNMLRQRVTGDYLQTSAILDRQFDVVSAVNDINDYQGPGTGYRISAERWAEIKNIAGVVQPGSIE

SEQ ID NO: 142 - protein, 194 aa, Citrobacter freundii
MECTTERKPVFTLQVSEGEAAKADERVDEVVIGVGPAFDKYQHKTLIDMPHKAILKELVAGIEEEGLHARVVRILRTSDVSFMAWDAANLSGSGIGIGIQSKGTTVIHQRDLLPLSNLELFSQAPLLTLETYRQIGKNAARYARKESPSPVPVVNDQMVRPKFMAKAALFHIKETKHVVQDRAPVTLHIALVRE

SEQ ID NO: 143 - protein, 142 aa, Citrobacter freundii
MNDNIMTAQDYPLATRCPEKIQTPTGKPLTEITLENVLAGRVGPQDVRISQQTLEYQAQIAEQMQRHAVARNFRRAAELIAIPDARILEIYNALRPFRSSFAELQAIADELEHTWHATVNAGFVRESAEVYLQRNKLRKGSQ

SEQ ID NO: 144 - DNA, 1359 bp, Artificial sequence
  Codon optimized Vibrio fluvialis aminepyruvate transaminase
atgaacaaaccacagtcttgggaagctcgtgcagaaacctattctctgtacggcttcactgacatgccgtccctgcaccagcgtggtactgttgttgtcacgcacggcgaaggtccgtacattgttgacgtcaatggtcgccgttatctggacgctaattctggcctgtggaatatggttgcaggttttgaccataagggtctgatcgacgcagctaaggctcagtacgagcgttttccgggctaccatgcgttcttcggtcgtatgagcgatcagacggtgatgctgtccgaaaaactggtagaagtctctccgttcgacagcggccgtgtgttctatacgaacagcggtagcgaagcaaacgacactatggttaagatgctgtggttcctgcatgcggcggaaggtaagccacaaaagcgcaaaattctgacccgttggaacgcgtatcacggcgttactgcagttagcgcctccatgaccggtaaaccgtacaacagcgttttcggtctgccgctgccaggtttcgttcacctgacttgccctcactactggcgttacggtgaagaaggcgagacggaagaacaattcgttgcacgcctggcacgcgaactggaagagactatccagcgtgagggtgctgacactatcgctggcttctttgctgagccggttatgggtgcaggtggtgttattccgcctgctaaaggttattttcaggctattctgccaatcctgcgtaaatatgacatcccggttatctctgacgaagttatctgtggttttggtcgcactggcaacacctggggttgcgtaacttatgattttactccggatgctatcatctctagcaaaaacctgaccgccggtttcttcccgatgggcgcagtgatcctgggtccagaactgagcaagcgcctggaaaccgcaattgaagcaatcgaggaatttccgcacggctttaccgcgtccggccatccggtaggctgtgcaatcgcgctgaaagcgatcgatgttgttatgaacgaaggcctggcggaaaacgttcgccgtctggcaccgcgcttcgaagaacgtctgaaacatatcgcggaacgtccgaacattggtgaatatcgtggtatcggttttatgtgggctctggaggcagtcaaagacaaagcgtctaaaactccgttcgatggcaatctgagcgtgagcgaacgtatcgccaacacttgcaccgacctgggtctgatctgccgtccactgggccaaagcgtagtgctgtgtccgccgtttatcctgaccgaagcgcaaatggacgaaatgttcgacaaactggagaaagcactggataaagtgttcgcagaggtggca

SEQ ID NO: 145 - DNA, 1668 bp, Klebsiella pneumoniae
atgaaaagatcaaaacgatttgcagtactggcccagcgccccgtcaatcaggacgggctgattggcgagtggcctgaagaggggctgatcgccatggacagcccctttgacccggtctcttcagtaaaagtggacaacggtctgatcgtcgaactggacggcaaacgccgggaccagtttgacatgatcgaccgatttatcgccgattacgcgatcaacgttgagcgcacagagcaggcaatgcgcctggaggcggtggaaatagcccgtatgctggtggatattcacgtcagccgggaggagatcattgccatcactaccgccatcacgccggccaaagcggtcgaggtgatggcgcagatgaacgtggtggagatgatgatggcgctgcagaagatgcgtgcccgccggaccccctccaaccagtgccacgtcaccaatctcaaagataatccggtgcagattgccgctgacgccgccgaggccgggatccgcggcttctcagaacaggagaccacggtcggtatcgcgcgctacgcgccgtttaacgccctggcgctgttggtcggttcgcagtgcggccgccccggcgtgttgacgcagtgctcggtggaagaggccaccgagctggagctgggcatgcgtggcttaaccagctacgccgagacggtgtcggtctacggcaccgaagcggtatttaccgacggcgatgatacgccgtggtcaaaggcgttcctcgcctcggcctacgcctcccgcgggttgaaaatgcgctacacctccggcaccggatccgaagcgctgatgggctattcggagagcaagtcgatgctctacctcgaatcgcgctgcatcttcattactaaaggcgccggggttcagggactgcaaaacggcgcggtgagctgtatcggcatgaccggcgctgtgccgtcgggcattcgggcggtgctggcggaaaacctgatcgcctctatgctcgacctcgaagtggcgtccgccaacgaccagactttctcccactcggatattcgccgcaccgcgcgcaccctgatgcagatgctgccgggcaccgactttattttctccggctacagcgcggtgccgaactacgacaacatgttcgccggctcgaacttcgatgcggaagattttgatgattacaacatcctgcagcgtgacctgatggttgacggcggcctgcgtccggtgaccgaggcggaaaccattgccattcgccagaaagcggcgcgggcgatccaggcggttttccgcgagctggggctgccgccaatcgccgacgaggaggtggaggccgccacctacgcgcacggcagcaacgagatgccgccgcgtaacgtggtggaggatctgagtgcggtggaagagatgatgaagcgcaacatcaccggcctcgatattgtcggcgcgctgagccgcagcggctttgaggatatcgccagcaatattctcaatatgctgcgccagcgggtcaccggcgattacctgcagacctcggccattctcgatcggcagttcgaggtggtgagtgcggtcaacgacatcaatgactatcaggggccgggcaccggctatcgcatctctgccgaacgctgggcggagatcaaaaatattccgggcgtggttcagcccgacaccattgaataa

SEQ ID NO: 146 - protein, 555 aa, Klebsiella pneumoniae
MKRSKRFAVLAQRPVNQDGLIGEWPEEGLIAMDSPFDPVSSVKVDNGLIVELDGKRRDQFDMIDRFIADYAINVERTEQAMRLEAVEIARMLVDIHVSREEIIAITTAITPAKAVEVMAQMNVVEMMMALQKMRARRTPSNQCHVTNLKDNPVQIAADAAEAGIRGFSEQETTVGIARYAPFNALALLVGSQCGRPGVLTQCSVEEATELELGMRGLTSYAETVSVYGTEAVFTDGDDTPWSKAFLASAYASRGLKMRYTSGTGSEALMGYSESKSMLYLESRCIFITKGAGVQGLQNGAVSCIGMTGAVPSGIRAVLAENLIASMLDLEVASANDQTFSHSDIRRTARTLMQMLPGTDFIFSGYSAVPNYDNMFAGSNFDAEDFDDYNILQRDLMVDGGLRPVTEAETIAIRQKAARAIQAVFRELGLPPIADEEVEAATYAHGSNEMPPRNVVEDLSAVEEMMKRNITGLDIVGALSRSGFEDIASNILNMLRQRVTGDYLQTSAILDRQFEVVSAVNDINDYQGPGTGYRISAERWAEIKNIPGVVQPDTIE

SEQ ID NO: 147 - DNA, 585 bp, Klebsiella pneumoniae
gtgcaacagacaacccaaattcagccctcttttaccctgaaaacccgcgagggcggggtagcttctgccgatgaacgcgccgatgaagtggtgatcggcgtcggccctgccttcgataaacaccagcatcacactctgatcgatatgccccatggcgcgatcctcaaagagctgattgccggggtggaagaagaggggcttcacgcccgggtggtgcgcattctgcgcacgtccgacgtctcctttatggcctgggatgcggccaacctgagcggctcggggatcggcatcggtatccagtcgaaggggaccacggtcatccatcagcgcgatctgctgccgctcagcaacctggagctgttctcccaggcgccgctgctgacgctggagacctaccggcagattggcaaaaacgctgcgcgctatgcgcgcaaagagtcaccttcgccggtgccggtggtgaacgatcagatggtgcggccgaaatttatggccaaagccgcgctatttcatatcaaagagaccaaacatgtggtgcaggacgccgagcccgtcaccctgcacatcgacttagtaagggagtga

SEQ ID NO: 148 - protein, 194 aa, Klebsiella pneumoniae
MQQTTQIQPSFTLKTREGGVASADERADEVVIGVGPAFDKHQHHTLIDMPHGAILKELIAGVEEEGLHARVVRILRTSDVSFMAWDAANLSGSGIGIGIQSKGTTVIHQRDLLPLSNLELFSQAPLLTLETYRQIGKNAARYARKESPSPVPVVNDQMVRPKFMAKAALFHIKETKHVVQDAEPVTLHIDLVRE

SEQ ID NO: 149 - DNA, 426 bp, Klebsiella pneumoniae
atgagcgagaaaaccatgcgcgtgcaggattatccgttagccacccgctgcccggagcatatcctgacgcctaccggcaaaccattgaccgatattaccctcgagaaggtgctctctggcgaggtgggcccgcaggatgtgcggatctcccgccagacccttgagtaccaggcgcagattgccgagcagatgcagcgccatgcggtggcgcgcaatttccgccgcgcggcggagcttatcgccattcctgacgagcgcattctggctatctataacgcgctgcgcccgttccgctcctcgcaggcggagctgctggcgatcgccgacgagctggagcacacctggcatgcgacagtgaatgccgcctttgtccgggagtcggcggaagtgtatcagcagcggcataagctgcgtaaaggaagctaa

SEQ ID NO: 150 - protein, 141 aa, Klebsiella pneumoniae
MSEKTMRVQDYPLATRCPEHILTPTGKPLTDITLEKVLSGEVGPQDVRISRQTLEYQAQIAEQMQRHAVARNFRRAAELIAIPDERILAIYNALRPFRSSQAELLAIADELEHTWHATVNAAFVRESAEVYQQRHKLRKGS

SEQ ID NO: 151 - DNA, 1824 bp, Klebsiella pneumoniae
atgccgttaatagccgggattgatatcggcaacgccaccaccgaggtggcgctggcgtccgactacccgcaggcgagggcgtttgttgccagcgggatcgtcgcgacgacgggcatgaaagggacgcgggacaatatcgccgggaccctcgccgcgctggagcaggccctggcgaaaacaccgtggtcgatgagcgatgtctctcgcatctatcttaacgaagccgcgccggtgattggcgatgtggcgatggagaccatcaccgagaccattatcaccgaatcgaccatgatcggtcataacccgcagacgccgggcggggtgggcgttggcgtggggacgactatcgccctcgggcggctggcgacgctgccggcggcgcagtatgccgaggggtggatcgtactgattgacgacgccgtcgatttccttgacgccgtgtggtggctcaatgaggcgctcgaccgggggatcaacgtggtggcggcgatcctcaaaaaggacgacggcgtgctggtgaacaaccgcctgcgtaaaaccctgccggtggtggatgaagtgacgctgctggagcaggtccccgagggggtaatggcggcggtggaagtggccgcgccgggccaggtggtgcggatcctgtcgaatccctacgggatcgccaccttcttcgggctaagcccggaagagacccaggccatcgtccccatcgcccgcgccctgattggcaaccgttccgcggtggtgctcaagaccccgcagggggatgtgcagtcgcgggtgatcccggcgggcaacctctacattagcggcgaaaagcgccgcggagaggccgatgtcgccgagggcgcggaagccatcatgcaggcgatgagcgcctgcgctccggtacgcgacatccgcggcgaaccgggcacccacgccggcggcatgcttgagcgggtgcgcaaggtaatggcgtccctgaccggccatgagatgagcgcgatatacatccaggatctgctggcggtggatacgtttattccgcgcaaggtgcagggcgggatggccggcgagtgcgccatggagaatgccgtcgggatggcggcgatggtgaaagcggatcgtctgcaaatgcaggttatcgcccgcgaactgagcgcccgactgcagaccgaggtggtggtgggcggcgtggaggccaacatggccatcgccggggcgttaaccactcccggctgtgcggcgccgctggcgatcctcgacctcggcgccggctcgacggatgcggcgatcgtcaacgcggaggggcagataacggcggtccatctcgccggggcggggaatatggtcagcctgttgattaaaaccgagctgggcctcgaggatctttcgctggcggaagcgataaaaaaatacccgctggccaaagtggaaagcctgttcagtattcgtcacgagaatggcgcggtggagttctttcgggaagccctcagcccggcggtgttcgccaaagtggtgtacatcaaggagggcgaactggtgccgatcgataacgccagcccgctggaaaaaattcgtctcgtgcgccggcaggcgaaagagaaagtgtttgtcaccaactgcctgcgcgcgctgcgccaggtctcacccggcggttccattcgcgatatcgcctttgtggtgctggtgggcggctcatcgctggactttgagatcccgcagcttatcacggaagccttgtcgcactatggcgtggtcgccgggcagggcaatattcggggaacagaagggccgcgcaatgcggtcgccaccgggctgctactggccggtcaggcgaattaa

SEQ ID NO: 152 - protein, 607 aa, Klebsiella pneumoniae
MPLIAGIDIGNATTEVALASDYPQARAFVASGIVATTGMKGTRDNIAGTLAALEQALAKTPWSMSDVSRIYLNEAAPVIGDVAMETITETIITESTMIGHNPQTPGGVGVGVGTTIALGRLATLPAAQYAEGWIVLIDDAVDFLDAVWWLNEALDRGINVVAAILKKDDGVLVNNRLRKTLPVVDEVTLLEQVPEGVMAAVEVAAPGQVVRILSNPYGIATFFGLSPEETQAIVPIARALIGNRSAVVLKTPQGDVQSRVIPAGNLYISGEKRRGEADVAEGAEAIMQAMSACAPVRDIRGEPGTHAGGMLERVRKVMASLTGHEMSAIYIQDLLAVDTFIPRKVQGGMAGECAMENAVGMAAMVKADRLQMQVIARELSARLQTEVVVGGVEANMAIAGALTTPGCAAPLAILDLGAGSTDAAIVNAEGQITAVHLAGAGNMVSLLIKTELGLEDLSLAEAIKKYPLAKVESLFSIRHENGAVEFFREALSPAVFAKVVYIKEGELVPIDNASPLEKIRLVRRQAKEKVFVTNCLRALRQVSPGGSIRDIAFVVLVGGSSLDFEIPQLITEALSHYGVVAGQGNIRGTEGPRNAVATGLLLAGQAN

SEQ ID NO: 153 - DNA, 354 bp, Klebsiella pneumoniae
atgtcgctttcaccgccaggcgtacgcctgttttacgatccgcgcgggcaccatgccggcgccatcaatgagctgtgctgggggctggaggagcagggggtcccctgccagaccataacctatgacggaggcggtgacgccgctgcgctgggcgccctggcggccagaagctcgcccctgcgggtgggtatcgggctcagcgcgtccggcgagatagccctcactcatgcccagctgccggcggacgcgccgctggctaccggacacgtcaccgatagcgacgatcaactgcgtacgctcggcgccaacgccgggcagctggttaaagtcctgccgttaagtgagagaaactga

SEQ ID NO: 154 - protein, 117 aa, Klebsiella pneumoniae
MSLSPPGVRLFYDPRGHHAGAINELCWGLEEQGVPCQTITYDGGGDAAALGALAARSSPLRVGIGLSASGEIALTHAQLPADAPLATGHVTDSDDQLRTLGANAGQLVKVLPLSERN

SEQ ID NO: 155 - DNA, 1125 bp, artificial sequence
  Codon optimized amino alcohol kinase from Erwinia caratovora subsp. atroseptica
atgagcgatggccgtctgaccgcactgtttcctgcatttccacatccggcatccaaccagccagtgtttgcggaggcttccccgcacgacgatgaactgatgacgcaggcggtgccgcaggtttcctgccagcaagccctggcaattgcccagcaggaatatggcctgagcggtcagatgagcctgctgcagggcgaacgtgacgttaatttctgtctgaccgtaacgccagatgaacgctatatgctgaaagtcatcaacgctgctgaaccggcagatgtgagcaactttcagactgcgctgctgctgcacctggcacgtcaggcgccagaactgccagtccctcgtatccgctccacgaaggctggtcagtctgaaacgggcgtcgaaattgatggtgttctgctgcgtgtgcgtctggtttcctacctggctggcatgccgcagtacctggcgtctccgagcacggcactgatgccacagctgggcggtactctggcgcagctggacaacgctctgcactctttcacccatccggcggctaaccgtgctctgctgtgggacatctcccgcgcagagcaggtccgcccgtacctggacttcgttagcgagccgcagcagtatcagcacctgcagcgcatctttgatcgctatgactctaacgtggcaccgctgctgacgacgctgcgccgccaggttatccacaacgacctgaacccgcataacgtcctggtcgatggttccagcccgacgcgcgtcacgggtatcatcgacttcggcgatgcagtgttcgcgccgctgatctgtgaggttgcgaccgctctggcgtaccaaattggcgacggcacggatctgctggaacatgtggtaccgtttgtcgcagcgtatcaccagcgtattccgctggcgccggaggaaatcgccctgctgccagatctgatcgcgacccgcatggcactgactctgaccatcgctcagtggcgtgcgtctcgctacccagataaccgcgaatacctgctgcgcaacgtgccgcgctgctggcactccctgcagcgtatcgcaacttacagccacgcacaatttctgacgcgcctgcagcaggtttgcccagaaaacgctcgttga

SEQ ID NO: 156 - DNA, 1275 bp, artificial sequence
  Codon optimized amino alcohol O-phosphate lyase from Erwinia caratovora subsp. atroseptica
atgactgcaactgaagctctgctggcacgtcgtcagcgcgttctgggcggtggctaccgtctgttctacgaagaaccgctgcatgttgcacgcggcgaaggtgtatggctgttcgatcatcagggtaaacgttacctggacgtatataacaacgtagctagcgtaggtcactgtcacccggccgttgtagaagcggtcgcgcgtcaatctgcgcaactgaacacccatacgcgctacctgcatcacgcgatcgtagattttgctgaagatctgctgtctgagttcccggcagaactgaacaacgtcatgctgacctgtactggctccgaagcgaacgacctggccctgcgcattgcgcgtcacgttacgggtggtaccggcatgctggtgacccgttgggcctaccatggtgttacgtccgctctggcggagctgtccccgtccctgggcgacggcgtagtacgcggttcccacgtaaagctgatcgatgctccggatacctaccgtcagccgggtgctttcctgacctctatccgcgaagcgctggcacagatgcagcgtgaaggtattcgtccggcggctctgctggttgatactatcttctcctccgacggtgtattctgtgcgccggaaggtgagatggcccaggcagccgcactgatccgtcaggccggtggcctgttcattgcggacgaagtgcagccgggctttggtcgtaccggtgaatccctgtggggtttcgcacgtcataacgtggttccagatctggtttctctgggcaaaccgatgggtaacggccatccgattgctggtctggtaggtcgctccgcactgttcgacgcttttggtcgtgatgttcgctactttaatactttcggcggtaacccagtatcctgccaggcggcacatgctgttctgcgcgttatccgtgaagaacagctgcagcagaacgcgcagcgtgttggtgattatctgcgccaaggtctgcagcagctggcacaacacttcccgctgatcggtgacattcgtgcatatggtctgtttatcggtgctgaactggtttccgaccgtgaatccaaaaccccagcgagcgagtctgcactgcaggttgttaacgcgatgcgtcagcgtggtgtactgatctccgcaaccggcccggcggcgaacattctgaagatccgtcctccgctggtattcctggaggaacacgcggacgtgttcctgactaccctgtccgacgtgctggcgctgatcggtactcgtgcacagcgttaa

SEQ ID NO: 157 - DNA, 77 bp, artificial sequence
  Primer
caggaggaattaaccatggggggttctcatcatcatcatcatcatggtgacgatgacgataagatgagcgatggccg

SEQ ID NO: 158 - DNA, 77 bp, artificial sequence
  Primer
cggccatcgctcatcttatcgtcatcgtcaccatgatgatgatgatgatgagaaccccccatggttaattcctcctg

SEQ ID NO: 159 - DNA, 20 bp, artificial sequence
  Primer
ggacctgcttcgctttatcg

SEQ ID NO: 160 - DNA, 15 bp, artificial sequence
  Primer
gctagagatgatagc

SEQ ID NO: 161 - DNA, 18 bp, artificial sequence
  Primer
ggaagagactatccagcg

SEQ ID NO: 162 - DNA, 50 bp, artificial sequence
  Primer
gcgcgcccgggaagaaggagctcttcaccatgaacaaaccacagtcttgg

SEQ ID NO: 163 - DNA, 28 bp, artificial sequence
  Primer
gcgcgcccgggttcatgccacctctgcg

SEQ ID NO: 164 - DNA, 2432 bp, Erwinia caratovora subsp. atroseptica
atgtctgacggacgactcaccgcactttttcctgcattcccacacccggcgtccaatcagcccgtatttgccgaggcttcaccgcacgacgacgagttaatgacgcaggccgtaccgcaggtttcctgtcagcaggcgttggcgattgcgcagcaagaatatggcttgtctgggcagatgtcgctgcttcagggcgagcgtgatgtgaatttctgtctgacggtgacgccagatgaacgctacatgctgaaagtcatcaatgcggcagaacctgccgacgtcagcaatttccaaaccgcgctgctgctgcatcttgcccgtcaggcacctgaactgcccgtaccgcgtatcaggtcgacaaaagcgggtcagtcggaaacaggcgttgagatcgatggtgtactgctgcgtgtgcggcttgtgagctatctggcaggaatgccgcagtatctggcctcaccgtcaacggcgctgatgccgcagttggggggaacgctggcgcagttggataacgcgcttcacagctttacgcatccggcggcaaaccgtgcgctgctgtgggatatcagccgggcagagcaggtgcgtccttacctcgatttcgtttctgaaccgcagcagtatcagcatcttcagcgtatttttgaccgttatgacagtaacgttgctcctctgttgacgacgctacgtcgtcaggtcattcataacgatctgaatccgcataacgtgctggtggatggatcgtcgccgacgcgggttactggcattatcgattttggcgatgccgtatttgccccgttaatttgcgaagtcgcgacggcactggcgtatcagatcggcgatggaaccgatttgttggagcatgttgtgccgtttgttgcggcctatcaccaacgcattccgttagcaccggaggagattgcgctgttacccgatctgatagcgacccgtatggcgctgaccctgaccattgcgcagtggcgagcatcgcgttatcccgacaatcgggagtatctgctgcgtaacgtgccgcgctgttggcacagtttgcagcgcattgcgacctattcccatgcgcaatttttgactcgcctacagcaggtttgcccggagaatgcgcgatgaaccagaaaggaatgacgtctatgacgtctgaaatgacagcgacagaagctttgctggcgcgccgtcagcgagtgttgggcggcggttatcgcctgttttatgaagagccgctgcatgtcgcgcgcggcgagggcgtgtggctgttcgatcaccaagggaaacgttatctggatgtctacaataatgtggcttcggtcggacattgccaccccgcggtggttgaagccgtggcgcgacagagcgcacaactcaatacccacacgcgctatttgcaccacgcgattgtcgattttgcggaagatttgctgagcgaatttcccgccgaattgaacaatgtaatgctgacctgtaccggcagtgaggctaacgatctggcgctgcgtatcgcccgacatgtcacgggcgggacggggatgttggtgacgcgctgggcgtatcacggcgtgaccagcgcgctggcggaactgtctccgtcgctgggggatggcgttgtgcgcggtagccatgtgaagctgatcgacgcgccagacacttatcgtcagcccggtgcatttcttaccagcattcgtgaagcgctggcgcagatgcaacgggaaggtattcgtcctgcggcgctgctggtagataccattttttccagcgatggcgtgttctgtgcgccggaaggcgaaatggcacaggcggcggcgttgatccgtcaggcgggcgggctgtttattgcggatgaagtgcagccgggcttcgggcgcaccggggaatcactgtggggctttgcgcgccacaatgtcgtccctgatttggtgagtctagggaaaccgatgggcaacggacatcccatcgctggattggtggggcgttccgctctgttcgacgcatttgggcgcgatgtgcgctatttcaatacctttggcggcaatccggtttcctgtcaggcggcgcacgcggtgctgcgggtgattcgggaagagcagttgcagcagaatgcccagcgggtcggtgattatctgcggcaagggttgcagcaactggcgcagcatttcccgctgattggtgatattcgggcttacggcctgtttattggtgcggagctggtcagcgatcgcgaaagtaaaacgccggcaagtgaatccgcgttgcaggtggtgaatgcgatgcgccaacgtggtgtgctcatcagcgcgacggggccagcggcgaacatactgaaaattcgcccgccgctggtgtttctggaagaacacgccgatgtgttcttaaccacgctgagtgacgttttagcgctcatcggcactcgtgcacagagataa

(19) United States
(12) Patent Application Publication
(10) Pub. No.: US 2008/0274522 A1
(43) Pub. Date: Nov. 6, 2008

(54) METHOD FOR THE PRODUCTION OF 2-BUTANONE

(76) Inventors: Michael G. Bramucci, Boothwyn, PA (US); Dennis Flint, Newark, DE (US); Edward S. Miller, Knoxville, TN (US); Vasantha Nagarajan, Wilmington, DE (US); Natalia Sedkova, Cherry Hill, NJ (US); Manjari Singh, West Chester, PA (US); Tina K. Van Dyk, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 12/110,514

(22) Filed: Apr. 28, 2008

Related U.S. Application Data
(60) Provisional application No. 60/915,465, filed on May 2, 2007.

(51) Int. Cl. C12P 7/26

(52) U.S. Cl. 435/148

(57) ABSTRACT

A method for the production of 2-butanone by fermentation using a microbial production host is disclosed. The method employs a reduction in temperature during the fermentation process that results in a more robust tolerance of the production host to the butanone product.

### FIELD OF THE INVENTION

The invention relates to a method for the production of 2-butanone by fermentation using a recombinant microbial host. Specifically, the method employs a decrease in temperature during fermentation that results in more robust tolerance of the production host to the 2-butanone product.

### BACKGROUND OF THE INVENTION

2-Butanone, also referred to as methyl ethyl ketone (MEK), is a widely used solvent and is the most important commercially produced ketone, after acetone. It is used as a solvent for paints, resins, and adhesives, as well as a selective extractant and activator of oxidative reactions.

Methods for the chemical synthesis of 2-butanone are known, such as by dehydrogenation of 2-butanol, or in a process where liquid butane is catalytically oxidized giving 2-butanone and acetic acid (Ullmann's Encyclopedia of Industrial Chemistry, 6^{th} edition, 2003, Wiley-VCHVerlag GmbH and Co., Weinheim, Germany, Vol. 5, pp. 727-732). 2-Butanone may also be converted chemically to 2-butanol by hydrogenation (Breen et al., J. or Catalysis 236: 270-281 (2005)). Butanol is an important industrial chemical, useful as a fuel additive, as a feedstock chemical in the plastics industry, and as a foodgrade extractant in the food and flavor industry. Each year 10 to 12 billion pounds of butanol are produced by petrochemical means and the need for this commodity chemical will likely increase.

The processes for chemical synthesis of 2-butanone use starting materials derived from petrochemicals and are generally expensive, and are not environmentally friendly. The production of 2-butanone from plant-derived raw materials would minimize green house gas emissions and would represent an advance in the art.

2-butanone is an intermediate in the production of 2-butanol by certain strains of Lactobacilli (Speranza et. al. J. Agric. Food Chem. (1997) 45:3476-3480). The substrate meso-2,3-butanediol is dehydrated to produce 2-butanone, which is hydrogenated to produce 2-butanol. The production of 2-butanol from acetolactate and acetoin by these Lactobacilli strains was also demonstrated.

Recombinant microbial production hosts expressing 2-butanone biosynthetic pathways are described in co-pending and commonly owned U.S. Patent Application Publication 20070259410A1. However, biological production of 2-butanone is believed to be limited by 2-butanone toxicity to the host microorganism used in the fermentation.

Some microbial strains that are tolerant to 2-butanone are known in the art (co-pending and commonly owned U.S. patent application Ser. No. 11/761,497 and Publication No. 20070259411). However, biological methods of producing 2-butanone to higher levels are required for cost effective commercial production.

There have been reports describing the effect of temperature on the tolerance of some microbial strains to ethanol. For example, Amartey et al. (Biotechnol. Lett. 13(9):627-632 (1991)) disclose that Bacillus stearothermophillus is less tolerant to ethanol at 70° C. than at 60° C. Herrero et al. (Appl. Environ. Microbiol. 40(3):571-577 (1980)) report that the optimum growth temperature of a wild-type strain of Clostridium thermocellum decreases as the concentration of ethanol challenge increases, whereas the optimum growth temperature of an ethanol-tolerant mutant remains constant. Brown et al. (Biotechnol. Lett. 4(4):269-274 (1982)) disclose that the yeast Saccharomyces uvarum is more resistant to growth inhibition by ethanol at temperatures 5° C. and 10° C. below its growth optimum of 35° C. However, fermentation became more resistant to ethanol inhibition with increasing temperature. Additionally, Van Uden (CRC Crit. Rev. Biotechnol. 1(3):263-273 (1984)) report that ethanol and other alkanols depress the maximum and the optimum growth temperature for growth of Saccharomyces cerevisiae while thermal death is enhanced. Moreover, Lewis et al. (U.S. Patent Application Publication No. 2004/0234649) describe methods for producing high levels of ethanol during fermentation of plant material comprising decreasing the temperature during saccharifying, fermenting, or simultaneously saccharifying and fermenting.

There have been a few reports on the effect of temperature on the tolerance of microbial strains to butanols. Harada (Hakko Kyokaishi 20:155-156 (1962)) discloses that the yield of 1-butanol in acetone-butanol-ethanol (ABE) fermentation is increased from 18.4%-18.7% to 19.1%-21.2% by lowering the temperature from 30° C. to 28° C. when the growth of the bacteria reaches a maximum. Jones et al. (Microbiol. Rev. 50(4):484-524 (1986)) review the role of temperature in ABE fermentation. They report that the solvent yields of three different solvent producing strains remains fairly constant at 31% at 30° C. and 33° C., but decreases to 23 to 25% at 37° C. Similar results were reported for Clostridium acetobutylicum for which solvent yields decreased from 29% at 25° C. to 24% at 40° C. In the latter case, the decrease in solvent yield was attributed to a decrease in acetone production while the yield of 1-butanol was unaffected. However, Carnarius (U.S. Pat. No. 2,198,104) reports that an increase in the butanol ratio is obtained in the ABE process by decreasing the temperature of the fermentation from 30° C. to 24° C. after 16 hours. However, the effect of temperature on the production of 2-butanone by recombinant microbial hosts is not known in the art.

There is a need, therefore, for a cost-effective process for the production of 2-butanone by fermentation that provides higher yields than processes known in the art. The present invention addresses this need through the discovery of a method for producing 2-butanone by fermentation using a recombinant microbial host, which employs a decrease in temperature during fermentation, resulting in more robust tolerance of the production host to the 2-butanone product.

### SUMMARY OF THE INVENTION

The invention provides a method for the production of 2-butanone by fermentation using a recombinant microbial host, which employs a decrease in temperature during fermentation that results in more robust tolerance of the production host to the 2-butanone product.

Accordingly, the invention provides a method for the production of 2-butanone comprising:
a) providing a recombinant microbial production host which produces 2-butanone;
b) seeding the production host of (a) into a fermentation medium comprising a fermentable carbon substrate to create a fermentation culture;
c) growing the production host in the fermentation culture at a first temperature for a first period of time;
d) lowering the temperature of the fermentation culture to a second temperature; and
e) incubating the production host at the second temperature of step (d) for a second period of time;
whereby 2-butanone is produced.

### BRIEF DESCRIPTION OF THE FIGURES AND SEQUENCE DESCRIPTIONS

The invention can be more fully understood from the following detailed description, figure, and the accompanying sequence descriptions, which form a part of this application.

FIG. 1 shows four different pathways for biosynthesis of 2-butanone and 2-butanol.

The following sequences conform with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37C.F.R. §1.822.

TABLE 1
Summary of Nucleic Acid and Protein SEQ ID Numbers

| | SEQ ID | |
|---|---|---|
| | Nucleic | SEQ ID |
| Description | acid | Protein |
| budA, acetolactate decarboxylase from Klebsiella | 1 | 2 |
| pneumoniae ATCC 25955 | | |
| alsD, acetolactate decarboxylase from Bacillus | 80 | 81 |
| subtilis | | |
| budA, acetolactate decarboxylase from Klebsiella | 82 | 83 |
| terrigena | | |
| budB, acetolactate synthase from Klebsiella | 3 | 4 |
| pneumoniae ATCC 25955 | | |
| alsS, acetolactate synthase from Bacillus subtilis | 76 | 77 |
| budB, acetolactate synthase from Klebsiella | 78 | 79 |
| terrigena | | |
| budC butanediol dehydrogenase from Klebsiella | 5 | 6 |
| pneumoniae IAM1063 | | |
| butanediol dehydrogenase from Bacillus cereus | 84 | 85 |
| butanediol dehydrogenase from Bacillus cereus | 86 | 87 |
| butB, butanediol dehydrogenase from Lactococcus | 88 | 89 |
| lactis | | |
| pddA, butanediol dehydratase alpha subunit from | 7 | 8 |
| Klebsiella oxytoca ATCC 8724 | | |
| pddB, butanediol dehydratase beta subunit from | 9 | 10 |
| Klebsiella oxytoca ATCC 8724 | | |
| pddC, butanediol dehydratase gamma subunit from | 11 | 12 |
| Klebsiella oxytoca ATCC 8724 | | |
| pduC, B12 dependent diol dehydratase large | 92 | 93 |
| subunit from Salmonella typhimurium | | |
| pduD, B12 dependent diol dehydratase medium | 94 | 95 |
| subunit from Salmonella typhimurium | | |
| pduE, B12 dependent diol dehydratase small | 96 | 97 |
| subunit from Salmonella typhimurium | | |
| pduC, B12 dependent diol dehydratase large | 98 | 99 |
| subunit from Lactobacillus collinoides | | |
| pduD, B12 dependent diol dehydratase medium | 100 | 101 |
| subunit from Lactobacillus collinoides | | |
| pduE, B12 dependent diol dehydratase small | 102 | 103 |
| subunit from Lactobacillus collinoides | | |
| pddC, adenosylcobalamin-dependent diol | 104 | 105 |
| dehydratase alpha subunit from Klebsiella | | |
| pneumoniae | | |
| pddD, adenosylcobalamin-dependent diol | 106 | 107 |
| dehydratase beta subunit from Klebsiella | | |
| pneumoniae | | |
| pddD, adenosylcobalamin-dependent diol | 108 | 109 |
| dehydratase gamma subunit from Klebsiella | | |
| pneumoniae | | |
| ddrA, diol dehydratase reactivating factor large | 110 | 111 |
| subunit from Klebsiella oxytoca | | |
| ddrB, diol dehydratase reactivating factor small | 112 | 113 |
| subunit from Klebsiella oxytoca | | |
| pduG, diol dehydratase reactivating factor large | 114 | 115 |
| subunit from Salmonella typhimurium | | |
| pduH, diol dehydratase reactivating factor small | 116 | 117 |
| subunit from Salmonella typhimurium | | |
| pduG, diol dehydratase reactivating factor large | 118 | 119 |
| subunit from Lactobacillus collinoides | | |
| pduH, diol dehydratase reactivating factor small | 120 | 121 |
| subunit from Lactobacillus collinoides | | |
| sadH, butanol dehydrogenase from Rhodococcus | 13 | 14 |
| ruber 219 | | |
| adhA, butanol dehydrogenase from Pyrococcus | 90 | 91 |
| furiosus | | |
| chnA, cyclohexanol dehydrogenase from | 71 | 72 |
| Acinteobacter sp. | | |
| yqhD, butanol dehydrogenase from Escherichia coli | 74 | 75 |
| amine:pyruvate transaminase from Vibrio fluvialis | 144 | 122 |
| (an acetoin aminase) | codon | |
| | opt. | |
| amino alcohol kinase from Erwinia carotovora | 123 | 124 |
| subsp. atroseptica | | |
| amino alcohol O-phosphate lyase from Erwinia | 125 | 126 |
| carotovora subsp. atroseptica | | |
| budC, acetoin reductase (butanediol | 133 | 134 |
| dehydrogenase) from Klebsiella terrigena (now | | |
| Raoultella terrigena) | | |
| glycerol dehydratase alpha subunit from Klebsiella | 145 | 146 |
| pneumoniae | | |
| glycerol dehydratase beta subunit from Klebsiella | 147 | 148 |
| pneumoniae | | |
| glycerol dehydratase gamma subunit from | 149 | 150 |
| Klebsiella pneumoniae | | |
| glycerol dehydratase reactivase large subunit from | 151 | 152 |
| Klebsiella pneumoniae | | |
| glycerol dehydratase reactivase small subunit from | 153 | 154 |
| Klebsiella pneumoniae | | |

SEQ ID NOs:15-65 are the nucleotide sequences of oligonucleotide PCR, cloning, screening, and sequencing primers used in the Examples.

SEQ ID NO:66 is nucleotide sequence of the deleted region of the yqhD gene in E. coli strain MG1655 ΔyqhCD, described in Example 15.

SEQ ID NO:67 is the nucleotide sequence of a variant of the glucose isomerase promoter 1.6Gl.

SEQ ID NO:68 is the nucleotide sequence of the 1.5Gl promoter.

SEQ ID NO:69 is the nucleotide sequence of the diol dehydratase operon from Klebsiella oxytoca.

SEQ ID NO:70 is the nucleotide sequence of the diol dehydratase reactivating factor operon from Klebsiella oxytoca.

SEQ ID NO:73 is the nucleotide sequence of pDCQ2, which is described in Example 13.

SEQ ID NOs:127-132 are the nucleotide sequences of additional oligonucleotide PCR and cloning primers used in the Examples.

SEQ ID NO:155 is a codon optimized coding region for the amino alcohol kinase of Erwinia carotovora subsp. atroseptica.

SEQ ID NO:156 is a codon optimized coding region for the amino alcohol O-phosphate lyase of Erwinia carotovora subsp. atroseptica.

SEQ ID NOs:157-163 are the nucleotide sequences of additional oligonucleotide PCR and cloning primers used in the Examples.

SEQ ID NO:164 is the nucleotide sequence of an operon from Erwinia carotovora subsp. atroseptica.

TABLE 2
Additional glycerol and diol dehydratase large, medium and small subunits

| | | protein |
|---|---|---|
| ^{a}Description | ^{b}subunit | SEQ ID |
| Corresponding subunits from same organism^{c} | | |
| Glycerol dehydratase alpha subunit from Clostridium | L | 135 |
| pasteurianum | | |
| Glycerol dehydratase beta subunit from Clostridium | M | 136 |
| pasteurianum | | |
| Glycerol dehydratase gamma subunit from Clostridium | S | 137 |
| pasteurianum | | |
| Glycerol dehydratase alpha subunit from Escherichia | L | 138 |
| blattae | | |
| Glycerol dehydratase beta subunit from Escherichia | M | 139 |
| blattae | | |
| Glycerol dehydratase gamma subunit from Escherichia | S | 140 |
| blattae | | |
| Glycerol dehydratase alpha subunit from Citrobacter | L | 141 |
| freundii | | |
| Glycerol dehydratase beta subunit from Citrobacter | M | 142 |
| freundii | | |
| Glycerol dehydratase gamma subunit from Citrobacter | S | 143 |
| freundii | | |

^{a}Description: from the Genbank annotation of the sequence and may not be correct including the glycerol or diol designation, or may not include subunit information.
^{b}Subunit: identified by sequence homology to the large, medium, or small subunit.of the Klebsiella oxytoca enzyme.
^{c}Subunts are listed together that are from the same organism and have annotations as the same enzyme, or have Genbank numbers close together indicating proximity in the genome.

### DETAILED DESCRIPTION OF THE INVENTION

The present invention relates to a method for the production of 2-butanone using recombinant microorganisms that employs a decrease in temperature during fermentation, resulting in more robust tolerance of the production host to the 2-butanone product and therefore a higher titer of 2-butanone. The present invention meets a number of commercial and industrial needs. 2-Butanone, also known as methyl ethyl ketone (MEK), is useful as a solvent in paints and other coatings. It is also used in the synthetic rubber industry and in the production of paraffin wax. 2-Butanone may also be converted chemically to 2-butanol by hydrogenation (Breen et al., J. or Catalysis 236: 270-281 (2005)). Butanol is an important industrial chemical, useful as a fuel additive, as a feedstock chemical in the plastics industry, and as a foodgrade extractant in the food and flavor industry. Each year 10 to 12 billion pounds of butanol are produced by petrochemical means and the need for this commodity chemical will likely increase.

The present invention produces 2-butanone from plant derived carbon sources, avoiding the negative environmental impact associated with standard petrochemical processes for 2-butanone production.

The following definitions and abbreviations are to be used for the interpretation of the claims and the specification.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the specification and the claims.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

The term “2-butanol biosynthetic pathway” refers to the enzyme pathways to produce 2-butanol from pyruvate.

The term “2-butanone biosynthetic pathway” refers to the enzyme pathways to produce 2-butanone from pyruvate.

The term “acetolactate synthase”, also known as “acetohydroxy acid synthase”, refers to a polypeptide (or polypeptides) having an enzyme activity that catalyzes the conversion of two molecules of pyruvic acid to one molecule of alpha-acetolactate. Acetolactate synthase, known as EC 2.2.1.6 [formerly 4.1.3.18] (Enzyme Nomenclature 1992, Academic Press, San Diego) may be dependent on the cofactor thiamin pyrophosphate for its activity. Suitable acetolactate synthase enzymes are available from a number of sources, for example, Bacillus subtilis [GenBank Nos: AAA22222 NCBI (National Center for Biotechnology Information) amino acid sequence (SEQ ID NO:77), L04470 NCBI nucleotide sequence (SEQ ID NO:76)], Klebsiella terrigena [GenBank Nos: AAA25055 (SEQ ID NO:79), L04507 (SEQ ID NO:78)], and Klebsiella pneumoniae [GenBank Nos: AAA25079 (SEQ ID NO:4), M73842 (SEQ ID NO:3)].

The term “acetolactate decarboxylase” refers to a polypeptide (or polypeptides) having an enzyme activity that catalyzes the conversion of alpha-acetolactate to acetoin. Acetolactate decarboxylases are known as EC 4.1.1.5 and are available, for example, from Bacillus subtilis [GenBank Nos: AAA22223 (SEQ ID NO:81), L04470 (SEQ ID NO:80)], Klebsiella terrigena [GenBank Nos: AAA25054 (SEQ ID NO:83), L04507 (SEQ ID NO:82)] and Klebsiella pneumoniae [GenBank Nos: AAU43774 (SEQ ID NO:2), AY722056 (SEQ ID NO:1)].

The term “acetoin aminase” refers to a polypeptide (or polypeptides) having an enzyme activity that catalyzes the conversion of acetoin to 3-amino-2-butanol. Acetoin aminase may utilize the cofactor pyridoxal 5′-phosphate or NADH (reduced nicotinamide adenine dinucleotide) or NADPH (reduced nicotinamide adenine dinucleotide phosphate). The resulting product may have (R) or (S) stereochemistry at the 3-position. The pyridoxal phosphate-dependent enzyme may use an amino acid such as alanine or glutamate as the amino donor. The NADH- and NADPH-dependent enzymes may use ammonia as a second substrate. A suitable example of an NADH-dependent acetoin aminase, also known as amino alcohol dehydrogenase, is described by Ito et al. (U.S. Pat. No. 6,432,688). An example of a pyridoxal-dependent acetoin aminase is the amine:pyruvate aminotransferase (also called amine:pyruvate transaminase) described by Shin and Kim (J. Org. Chem. 67:2848-2853 (2002)).

The term “butanol dehydrogenase” refers to a polypeptide (or polypeptides) having an enzyme activity that catalyzes the interconversion of 2-butanone and 2-butanol. Butanol dehydrogenases are a subset of a broad family of alcohol dehydrogenases. Butanol dehydrogenase may be NAD- or NADP-dependent. The NAD-dependent enzymes are known as EC 1.1.1.1 and are available, for example, from Rhodococcus ruber [GenBank Nos: CAD36475 (SEQ ID NO:14), AJ491307 (SEQ ID NO:13)]. The NADP-dependent enzymes are known as EC 1.1.1.2 and are available, for example, from Pyrococcus furiosus [GenBank Nos: AAC25556 (SEQ ID NO:91), AF013169 (SEQ ID NO:90)]. Additionally, a butanol dehydrogenase is available from Escherichia coli [GenBank Nos:NP_{—}417-484 (SEQ ID NO:75), NC-000913 (SEQ ID NO:74)] and a cyclohexanol dehydrogenase with activity towards 2-butanol is available from Acinetobacter sp. [GenBank Nos: AAG10026 (SEQ ID NO:72), AF282240 (SEQ ID NO:71)].

The term “acetoin kinase” refers to a polypeptide (or polypeptides) having an enzyme activity that catalyzes the conversion of acetoin to phosphoacetoin. Acetoin kinase may utilize ATP (adenosine triphosphate) or phosphoenolpyruvate as the phosphate donor in the reaction. Although there are no reports of enzymes catalyzing this reaction on acetoin, there are enzymes that catalyze the analogous reaction on the similar substrate dihydroxyacetone, for example, enzymes known as EC 2.7.1.29 (Garcia-Alles et al. (2004) Biochemistry 43:13037-13046).

The term “acetoin phosphate aminase” refers to a polypeptide (or polypeptides) having an enzyme activity that catalyzes the conversion of acetoin phosphate (also called phosphoacetoin) to 3-amino-2-butanol O-phosphate. Acetoin phosphate aminase may use the cofactor pyridoxal 5′-phosphate, NADH or NADPH. The resulting product may have (R) or (S) stereochemistry at the 3-position. The pyridoxal phosphate-dependent enzyme may use an amino acid such as alanine or glutamate. The NADH- and NADPH-dependent enzymes may use ammonia as a second substrate. Although there are no reports of enzymes catalyzing this reaction on acetoin phosphate, there is a pyridoxal phosphate-dependent enzyme that is proposed to carry out the analogous reaction on the similar substrate serinol phosphate (Yasuta et al. (2001) Appl. Environ. Microbiol. 67:4999-5009).

The term “aminobutanol phosphate phospho-lyase”, also called “amino alcohol O-phosphate lyase”, refers to a polypeptide (or polypeptides) having an enzyme activity that catalyzes the conversion of 3-amino-2-butanol O-phosphate to 2-butanone. Aminobutanol phosphate phospho-lyase may utilize the cofactor pyridoxal 5′-phosphate. There are reports of enzymes that catalyze the analogous reaction on the similar substrate 1-amino-2-propanol phosphate (Jones et al. (1973) Biochem J. 134:167-182). Disclosed in co-owned and co-pending US Patent Application Publication No. 20070259410A1 is an aminobutanol phosphate phospho-lyase (SEQ ID NO: 126) from the organism Erwinia carotovora, with demonstrated aminobutanol phosphate phospho-lyase activity.

The term “aminobutanol kinase” refers to a polypeptide (or polypeptides) having an enzyme activity that catalyzes the conversion of 3-amino-2-butanol to 3-amino-2-butanol O-phosphate. Aminobutanol kinase may utilize ATP as the phosphate donor. There are reports of enzymes that catalyze the analogous reaction on the similar substrates ethanolamine and 1-amino-2-propanol (Jones et al., supra). Disclosed in co-owned and co-pending US Patent Application Publication No. 20070259410A1 is an amino alcohol kinase of Erwinia carotovora subsp. atroseptica (SEQ ID NO:124).

The term “butanediol dehydrogenase” also known as “acetoin reductase” refers to a polypeptide (or polypeptides) having an enzyme activity that catalyzes the conversion of acetoin to 2,3-butanediol. Butanediol dehydrogenases are a subset of the broad family of alcohol dehydrogenases. Butanediol dehydrogenase enzymes may have specificity for production of (R)- or (S)-stereochemistry in the alcohol product. (S)-specific butanediol dehydrogenases are known as EC 1.1.1.76 and are available, for example, from Klebsiella pneumoniae (GenBank Nos: BBA13085 (SEQ ID NO:6), D86412 (SEQ ID NO:5)). (R)-specific butanediol dehydrogenases are known as EC 1.1.1.4 and are available, for example, from Bacillus cereus [GenBank Nos. NP_{—}830481 (SEQ ID NO:85), NC_{—}004722 (SEQ ID NO:84); AAP07682 (SEQ ID NO:87), AE017000 (SEQ ID NO:86)], and Lactococcus lactis [GenBank Nos. AAK04995 (SEQ ID NO:89), AE006323 (SEQ ID NO:88)].

The term “butanediol dehydratase”, also known as “diol dehydratase” or “propanediol dehydratase” refers to a polypeptide (or polypeptides) having an enzyme activity that catalyzes the conversion of 2,3-butanediol to 2-butanone. Butanediol dehydratase may utilize the cofactor adenosyl cobalamin (vitamin B12). Adenosyl cobalamin-dependent enzymes are known as EC 4.2.1.28 and are available, for example, from Klebsiella oxytoca [GenBank Nos: BAA08099 (alpha subunit) (SEQ ID NO:8), D45071 (SEQ ID NO:7); BAA08100 (beta subunit) (SEQ ID NO:10), D45071 (SEQ ID NO:9); and BBA08101 (gamma subunit) (SEQ ID NO:12), D45071 (SEQ ID NO:11) (Note all three subunits are required for activity)], and Klebsiella pneumoniae [GenBank Nos: AAC98384 (alpha subunit) (SEQ ID NO:105), AF102064 (SEQ ID NO:104); GenBank Nos: AAC98385 (beta subunit) (SEQ ID NO:107), AF102064 (SEQ ID NO:106), GenBank Nos: AAC98386 (gamma subunit) SEQ ID NO:109), AF102064 (SEQ ID NO:108)]. Other suitable diol dehydratases include, but are not limited to, B12-dependent diol dehydratases available from Salmonella typhimurium [GenBank Nos: AAB84102 (large subunit) (SEQ ID NO:93), AF026270 (SEQ ID NO:92); GenBank Nos: AAB84103 (medium subunit) (SEQ ID NO:95), AF026270 (SEQ ID NO:94); GenBank Nos: AAB84104 (small subunit) (SEQ ID NO:97), AF026270 (SEQ ID NO:96)]; and Lactobacillus collinoides [GenBank Nos: CAC82541 (large subunit) (SEQ ID NO:99), AJ297723 (SEQ ID NO:98); GenBank Nos: CAC82542 (medium subunit) (SEQ ID NO:101); AJ297723 (SEQ ID NO:100); GenBank Nos: CAD01091 (small subunit) (SEQ ID NO:103), AJ297723 (SEQ ID NO:102)]; and enzymes from Lactobacillus brevis (particularly strains CNRZ 734 and CNRZ 735, Speranza et al., supra), and nucleotide sequences that encode the corresponding enzymes. Methods of diol dehydratase gene isolation are well known in the art (e.g., U.S. Pat. No. 5,686,276).

The term “glycerol dehydratase” refers to a polypeptide (or polypeptides) having an enzyme activity that catalyzes the conversion of glycerol to 3-hydroxypropionaldehyde. Adenosyl cobalamin-dependent glycerol dehydratases are known as EC 4.2.1.30. The glycerol dehydratases of EC 4.2.1.30 are similar to the diol dehydratases in sequence and in having three subunits. The glycerol dehydratases can also be used to convert 2,3-butanediol to 2-butanone. Some examples of glycerol dehydratases of EC 4.2.1.30 include those from Klebsiella pneumoniae (alpha subunit, SEQ ID NO:145, coding region and SEQ ID NO:146, protein; beta subunit, SEQ ID NO:147, coding region and SEQ ID NO:148, protein; and gamma subunit SEQ ID NO:149, coding region and SEQ ID NO:150, protein); from Clostridium pasteurianum [GenBank Nos: 3360389 (alpha subunit, SEQ ID NO:135), 3360390 (beta subunit, SEQ ID NO:136), and 3360391 (gamma subunit, SEQ ID NO:137)]; from Escherichia blattae [GenBank Nos: 60099613 (alpha subunit, SEQ ID NO:138), 57340191 (beta subunit, SEQ ID NO:139), and 57340192 (gamma subunit, SEQ ID NO:140)]; and from Citrobacter freundii [GenBank Nos: 1169287 (alpha subunit, SEQ ID NO:141), 1229154 (beta subunit, SEQ ID NO:142), and 1229155 (gamma subunit, SEQ ID NO:143)]. Note that all three subunits are required for activity. Additional glycerol dehydratases are listed in Table 2.

Diol and glycerol dehydratases may undergo suicide inactivation during catalysis. A reactivating factor protein, also referred to herein as “reactivase”, can be used to reactivate the inactive enzymes (Mori et al., J. Biol. Chem. 272:32034 (1997)). Preferably, the reactivating factor is obtained from the same source as the diol or glycerol dehydratase used. For example, suitable diol dehydratase reactivating factors are available from Klebsiella oxytoca [GenBank Nos: MC15871 (large subunit) (SEQ ID NO:111), AF017781 (SEQ ID NO:110); GenBank Nos: AAC15872 (small subunit) (SEQ ID NO:113), AF017781 (SEQ ID NO:112)]; Salmonella typhimurium [GenBank Nos: AAB84105 (large subunit) (SEQ ID NO:115), AF026270 (SEQ ID NO:114), GenBank Nos: AAD39008 (small subunit) (SEQ ID NO:117), AF026270 (SEQ ID NO:116)]; and Lactobacillus collinoides [GenBank Nos: CAD01092 (large subunit) (SEQ ID NO:119), AJ297723 (SEQ ID NO:118); GenBank Nos: CAD01093 (small subunit) (SEQ ID NO:121), AJ297723 (SEQ ID NO:120)]. Both the large and small subunits are required for activity. For example, suitable glycerol dehydratase reactivating factors are available from Klebsiella pneumoniae (large subunit, SEQ ID NO:151, coding region and SEQ ID NO:152, protein, and small subunit, SEQ ID NO:153, coding region and SEQ ID NO:154, protein).

The term “a facultative anaerobe” refers to a microorganism that can grow in both aerobic and anaerobic environments.

The term “carbon substrate” or “fermentable carbon substrate” refers to a carbon source capable of being metabolized by host organisms disclosed herein and particularly carbon sources selected from the group consisting of monosaccharides, oligosaccharides, polysaccharides, and one-carbon substrates or mixtures thereof.

The term “gene” refers to a nucleic acid fragment that is capable of being expressed as a specific protein, optionally including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” or “heterologous” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

As used herein, an “isolated nucleic acid fragment” or “isolated nucleic acid molecule” or “genetic construct” will be used interchangeably and will mean a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

A nucleic acid fragment is “hybridizable” to another nucleic acid fragment, such as a cDNA, genomic DNA, or RNA molecule, when a single-stranded form of the nucleic acid fragment can anneal to the other nucleic acid fragment under the appropriate conditions of temperature and solution ionic strength. Hybridization and washing conditions are well known and exemplified in Sambrook, J., Fritsch, E. F. and Maniatis, T. Molecular Cloning: A Laboratory Manual, 2 ed., Cold Spring Harbor Laboratory: Cold Spring Harbor, N.Y. (1989), particularly Chapter 11 and Table 11.1 therein (entirely incorporated herein by reference). The conditions of temperature and ionic strength determine the “stringency” of the hybridization. Stringency conditions can be adjusted to screen for moderately similar fragments (such as homologous sequences from distantly related organisms), to highly similar fragments (such as genes that duplicate functional enzymes from closely related organisms). Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. An additional set of stringent conditions include hybridization at 0.1×SSC, 0.1% SDS, 65° C. and washes with 2×SSC, 0.1% SDS followed by 0.1×SSC, 0.1% SDS, for example.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least about 30 nucleotides. Furthermore, the skilled artisan will recognize that the temperature and wash solution salt concentration may be adjusted as necessary according to factors such as length of the probe.

A “substantial portion” of an amino acid or nucleotide sequence is that portion comprising enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Altschul, S. F., et al., J. Mol. Biol., 215:403-410 (1993)). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence. The instant specification teaches the complete amino acid and nucleotide sequence encoding particular fungal proteins. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

The term “complementary” is used to describe the relationship between nucleotide bases that are capable of hybridizing to one another. For example, with respect to DNA, adenosine is complementary to thymine and cytosine is complementary to guanine.

The terms “homology” and “homologous” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences.

Moreover, the skilled artisan recognizes that homologous nucleic acid sequences encompassed by this invention are also defined by their ability to hybridize, under moderately stringent conditions (e.g., 0.5×SSC, 0.1% SDS, 60° C.) with the sequences exemplified herein, or to any portion of the nucleotide sequences disclosed herein and which are functionally equivalent to any of the nucleic acid sequences disclosed herein.

“Codon degeneracy” refers to the nature in the genetic code permitting variation of the nucleotide sequence without effecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: 1.) Computational Molecular Biology (Lesk, A. M., Ed.) Oxford University: NY (1988); 2.) Biocomputing: Informatics and Genome Projects (Smith, D. W., Ed.) Academic: NY (1993); 3.) Computer Analysis of Sequence Data, Part I (Griffin, A. M., and Griffin, H. G., Eds.) Humania: NJ (1994); 4.) Sequence Analysis in Molecular Biology (von Heinje, G., Ed.) Academic (1987); and 5.) Sequence Analysis Primer (Gribskov, M. and Devereux, J., Eds.) Stockton: NY (1991).

Preferred methods to determine identity are designed to give the best match between the sequences tested. Methods to determine identity and similarity are codified in publicly available computer programs. Sequence alignments and percent identity calculations may be performed using the MegAlign™ program of the LASERGENE bioinformatics computing suite (DNASTAR Inc., Madison, Wis.). Multiple alignment of the sequences is performed using the “Clustal method of alignment” which encompasses several varieties of the algorithm including the “Clustal V method of alignment” corresponding to the alignment method labeled Clustal V (described by Higgins and Sharp, CABIOS. 5:151-153 (1989); Higgins, D. G. et al., Comput. Appl. Biosci., 8:189-191 (1992)) and found in the MegAlign™ program of the LASERGENE bioinformatics computing suite (DNASTAR Inc.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program. Additionally the “Clustal W method of alignment” is available and corresponds to the alignment method labeled Clustal W (described by Higgins and Sharp, CABIOS. 5:151-153 (1989); Higgins, D. G. et al., Comput. Appl. Biosci. 8:189-191 (1992)) and found in the MegAlign™ v6.1 program of the LASERGENE bioinformatics computing suite (DNASTAR Inc.). Default parameters for multiple alignment (GAP PENALTY=10, GAP LENGTH PENALTY=0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program.

It is well understood by one skilled in the art that many levels of sequence identity are useful in identifying polypeptides, from other species, wherein such polypeptides have the same or similar function or activity. Useful examples of percent identities include, but are not limited to: 24%, 30%, 35%, 40%, 45%, 50%, 55%, 60%, 65%, 70%, 75%, 80%, 85%, 90%, or 95%, or any integer percentage from 24% to 100% may be useful in describing the present invention, such as 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. Suitable nucleic acid fragments not only have the above homologies but typically encode a polypeptide having at least 50 amino acids, preferably at least 100 amino acids, more preferably at least 150 amino acids, still more preferably at least 200 amino acids, and most preferably at least 250 amino acids.

The term “sequence analysis software” refers to any computer algorithm or software program that is useful for the analysis of nucleotide or amino acid sequences. “Sequence analysis software” may be commercially available or independently developed. Typical sequence analysis software will include, but is not limited to: 1.) the GCG suite of programs (Wisconsin Package Version 9.0, Genetics Computer Group (GCG), Madison, Wis.); 2.) BLASTP, BLASTN, BLASTX (Altschul et al., J. Mol. Biol., 215:403-410 (1990)); 3.) DNASTAR (DNASTAR, Inc. Madison, Wis.); 4.) Sequencher (Gene Codes Corporation, Ann Arbor, Mich.); and 5.) the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Plenum: New York, N.Y.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

As used herein the term “coding sequence” or “CDS” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure.

The term “promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of effecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment disclosed herein. Expression may also refer to translation of mRNA into a polypeptide.

As used herein the term “transformation” refers to the transfer of a nucleic acid fragment into a host organism, resulting in genetically stable inheritance. Host organisms containing the transformed nucleic acid fragments are referred to as “transgenic” or “recombinant” or “transformed” organisms.

The terms “plasmid” and “vector” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation vector” refers to a specific vector containing a foreign gene and having elements in addition to the foreign gene that facilitates transformation of a particular host cell.

As used herein the term “codon degeneracy” refers to the nature in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA.

The term “fermentation product medium” refers to a medium in which fermentation has occurred such that product is present in the medium.

Standard recombinant DNA and molecular cloning techniques used here are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Press, Cold Spring Harbor, N.Y. (1984); and by Ausubel, F. M. et al., Current Protocols in Molecular Biology, published by Greene Publishing Assoc. and Wiley-Interscience (1987).

### 2-Butanone Biosynthetic Pathways

Carbohydrate utilizing microorganisms employ the Embden-Meyerhof-Parnas (EMP) pathway, the Entner-Doudoroff pathway and the pentose phosphate cycle as the central, metabolic routes to provide energy and cellular precursors for growth and maintenance. These pathways have in common the intermediate glyceraldehyde 3-phosphate, and, ultimately, pyruvate is formed directly or in combination with the EMP pathway. The combined reactions of sugar conversion to pyruvate produce energy (e.g. adenosine 5′-triphosphate, ATP) and reducing equivalents (e.g. reduced nicotinamide adenine dinucleotide, NADH, and reduced nicotinamide adenine dinucleotide phosphate, NADPH). NADH and NADPH must be recycled to their oxidized forms (NAD⁺ and NADP⁺, respectively). In the presence of inorganic electron acceptors (e.g. O₂, NO₃⁻ and SO₄²⁻), the reducing equivalents may be used to augment the energy pool; alternatively, a reduced carbon by-product may be formed.

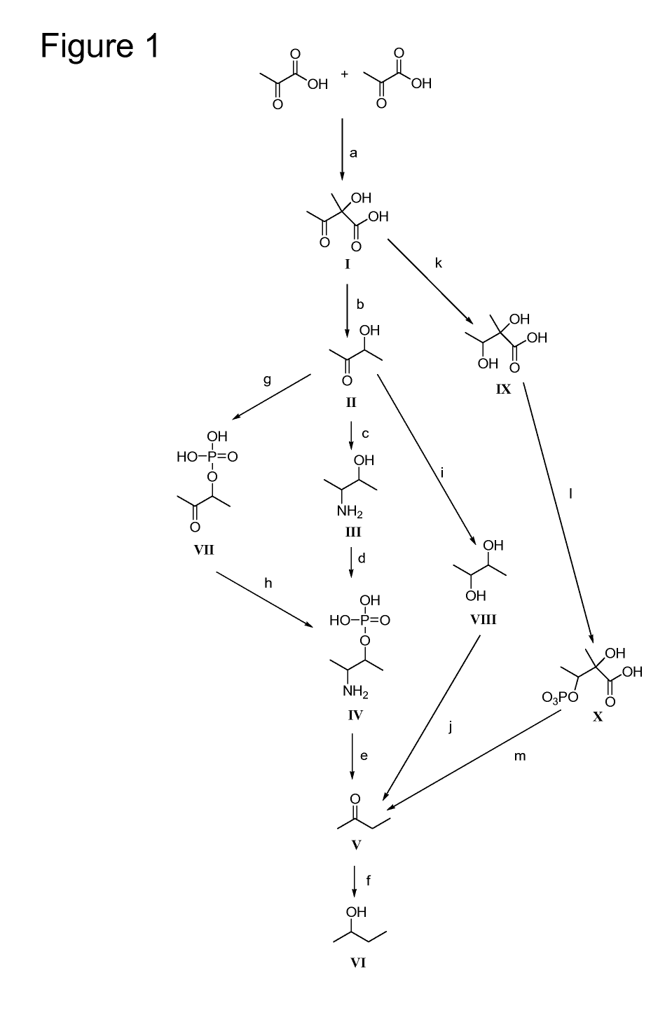

As described in co-owned and co-pending US Patent Application publications 20070259410A1 and 20070292927A1,2-butanone and 2-butanol can be produced from carbohydrate sources in recombinant microorganisms comprising a complete 2-butanone or 2-butanol biosynthetic pathway. Four biosynthetic pathways including all steps starting with pyruvate for production of 2-butanone or 2-butanol are shown in FIG. 1. The letters and roman numerals cited below correspond to the letters and roman numerals in FIG. 1, which are used to depict the conversion steps and products, respectively. As described below, 2-butanone is an intermediate in all of these 2-butanol biosynthetic pathways. 2-Butanone is the product when the last pathway step that converts 2-butanone to 2-butanol, by 2-butanol dehydrogenase, is omitted.

All of the pathways begin with the initial reaction of two pyruvate molecules to yield alpha-acetolactate (I), shown as the substrate to product conversion (a) in FIG. 1. From alpha-acetolactate, there are 4 possible pathways to 2-butanone (V), referred to herein as 2-butanone biosynthetic pathways:

Pathway
1) I--->II--->III--->IV--->V (substrate to product conversions b,c,d,e)
2) I--->II--->VII--->IV--->V (substrate to product conversions b,g,h,e)
3) I--->II--->VIII--->V (substrate to product conversions b,i,j)
4) I--->IX--->X--->V (substrate to product conversions k,l,m)
   A detailed discussion of the substrate to product conversions in each pathway is given below.

### Pathway 1:

### (a) pyruvate to alpha-acetolactate

The initial step in pathway 1 is the conversion of two molecules of pyruvate to one molecule of alpha-acetolactate (compound I in FIG. 1) and one molecule of carbon dioxide catalyzed by a thiamin pyrophosphate-dependent enzyme. Enzymes catalyzing this substrate to product conversion (generally called either acetolactate synthase or acetohydroxy acid synthase; EC 2.2.1.6 [switched from 4.1.3.18 in 2002]) are well-known, and they participate in the biosynthetic pathway for the proteinogenic amino acids leucine and valine, as well as in the pathway for fermentative production of 2,3-butanediol and acetoin of a number of organisms.

The skilled person will appreciate that polypeptides having acetolactate synthase activity isolated from a variety of sources will be useful in pathway 1 independent of sequence homology. Some examples of suitable acetolactate synthase enzymes are available from a number of sources, for example, Bacillus subtilis [GenBank Nos: AAA22222 NCBI (National Center for Biotechnology Information) amino acid sequence (SEQ ID NO:77), L04470 NCBI nucleotide sequence (SEQ ID NO:76)], Klebsiella terrigena [GenBank Nos: AAA25055 (SEQ ID NO:79), L04507 (SEQ ID NO:78)], and Klebsiella pneumoniae [GenBank Nos: AAA25079 (SEQ ID NO:4), M73842 (SEQ ID NO:3)]. Preferred acetolactate synthase enzymes are those that have at least 80%-85% identity to SEQ ID NO's 4, 77, and 79, where at least 85%-90% identity is more preferred and where at least 95% identity based on the Clustal W method of alignment using the default parameters of GAP PENALTY=10, GAP LENGTH PENALTY=0.1, and Gonnet 250 series of protein weight matrix, is most preferred.

### (b) alpha-acetolactate to acetoin

Alpha-acetolactate (I) is converted to acetoin (II) by the action of an enzyme such as acetolactate decarboxylase (EC 4.1.1.5). Like acetolactate synthase, this enzyme is thiamin pyrophosphate-dependent and is also involved in the production of 2,3-butanediol and acetoin by a number of organisms. The enzymes from different sources vary quite widely in size (25-50 kilodaltons), oligomerization (dimer-hexamer), localization (intracellular of extracellular), and allosteric regulation (for example, activation by branched-chain amino acids). An intracellular location is preferable to extracellular, but other variations are generally acceptable.

The skilled person will appreciate that polypeptides having acetolactate decarboxylase activity isolated from a variety of sources will be useful in pathway 1 independent of sequence homology. Some examples of suitable acetolactate decarboxylase enzymes are available from a number of sources, for example, Bacillus subtilis [GenBank Nos: AAA22223 (SEQ ID NO:81), L04470 (SEQ ID NO:80)], Klebsiella terrigena [GenBank Nos: AAA25054 (SEQ ID NO:83), L04507 (SEQ ID NO:82)] and Klebsiella pneumoniae [GenBank Nos: AAU43774 (SEQ ID NO:2), AY722056 (SEQ ID NO:1)].

Preferred acetolactate decarboxylase enzymes are those that have at least 80%-85% identity to SEQ ID NO's 2, 81 and 83, where at least 85%-90% identity is more preferred and where at least 95% identity based on the Clustal W method of alignment using the default parameters of GAP PENALTY=10, GAP LENGTH PENALTY=0.1, and Gonnet 250 series of protein weight matrix, is most preferred.

### (c) acetoin to 3-amino-2-butanol

There are two known types of biochemical reactions that could effect the substrate to product conversion of acetoin (II) to 3-amino-2-butanol (III), specifically, pyridoxal phosphate-dependent transamination utilizing an accessory amino donor and direct reductive amination with ammonia. In the latter case, the reducing equivalents are supplied in the form of a reduced nicotinamide cofactor (either NADH or NADPH). An example of an NADH-dependent enzyme catalyzing this reaction with acetoin as a substrate is reported by Ito et al. (U.S. Pat. No. 6,432,688). Any stereospecificity of this enzyme has not been assessed. An example of a pyridoxal phosphate-dependent transaminase that catalyzes the conversion of acetoin to 3-amino-2-butanol has been reported by Shin and Kim (supra). This enzyme was shown in co-owned and co-pending US Patent Application Publication No. 20070259410A1 to convert both the (R) isomer of acetoin to the (2R,3S) isomer of 3-amino-2-butanol and the (S) isomer of acetoin to the (2S,3S) isomer of 3-amino-2-butanol. Either type of enzyme (i.e., transaminase or reductive aminase) is considered to be an acetoin aminase and may be utilized in the production of 2-butanol. Other enzymes in this group may have different stereospecificities.

The skilled person will appreciate that polypeptides having acetoin aminase activity isolated from a variety of sources will be useful in the present invention independent of sequence homology. One example of a protein having this activity is described in co-owned and co-pending US Patent Application Publication No. 20070259410A1 (SEQ ID NO:122). Accordingly preferred acetoin aminase enzymes are those that have at least 80%-85% identity to SEQ ID NO:122, where at least 85%-90% identity is more preferred and where at least 95% identity based on the Clustal W method of alignment using the default parameters of GAP PENALTY=10, GAP LENGTH PENALTY=0.1, and Gonnet 250 series of protein weight matrix, is most preferred.

### (d) 3-amino-2-butanol to 3-amino-2-butanol O-phosphate

There are no enzymes known in the art that catalyze the substrate to product conversion of 3-amino-2-butanol (III) to 3-amino-2-butanol phosphate (IV). However, a few Pseudomonas and Erwinia species have been shown to express an ATP-dependent ethanolamine kinase (EC 2.7.1.82) which allows them to utilize ethanolamine or 1-amino-2-propanol as a nitrogen source (Jones et al. (1973) Biochem. J. 134:167-182). It is likely that this enzyme also has activity towards 3-amino-2-butanol or could be engineered to do so, thereby providing an aminobutanol kinase. Disclosed in co-owned and co-pending US Patent Application Publication No. 20070259410A1 is a gene of Erwinia carotovora subsp. atroseptica (SEQ ID NO:123) that encodes a protein (SEQ ID NO:24) identified as an amino alcohol kinase. This enzyme may be used to convert 3-amino-2-butanol to 3-amino-2-butanol O-phosphate.

The skilled person will appreciate that polypeptides having aminobutanol kinase activity isolated from a variety of sources will be useful in the present invention independent of sequence homology. One example of this activity is described in co-owned and co-pending US Patent Application Publication No. 20070259410A1 (SEQ ID NO:124). Accordingly preferred aminobutanol kinase enzymes are those that have at least 80%-85% identity to SEQ ID NO:124, where at least 85%-90% identity is more preferred and where at least 95% identity based on the Clustal W method of alignment using the default parameters of GAP PENALTY=10, GAP LENGTH PENALTY=0.1, and Gonnet 250 series of protein weight matrix, is most preferred.

### (e) 3-amino-2-butanol phosphate to 2-butanone

Although there are no enzymes reported to catalyze the substrate to product conversion of 3-amino-2-butanol phosphate (IV) to 2-butanone (V), the substrate is very similar to those utilized by the pyridoxal phosphate-dependent phosphoethanolamine phospho-lyase enzyme, which has been found in a small number of Pseudomonas and Erwinia species. These enzymes have activity towards phosphoethanolamine and both enantiomers of 2-phospho-1-aminopropane (Jones et al. (1973) Biochem. J. 134:167-182), and may also have activity towards 3-amino-2-butanol O-phosphate. Applicants have identified a gene of Erwinia carotovora subsp. atroseptica (SEQ ID NO:125) that encodes a protein (SEQ ID NO:126) with homology to class III aminotransferases was identified. It was shown to have activity on both aminopropanol phosphate and aminobutanol phosphate substrates. The enzyme was able to catalyze the conversion of a mixture of (R)-3-amino-(S)-2-butanol and (S)-3-amino-(R)-2-butanol O-phosphate, and a mixture of (R)-3-amino-(R)-2-butanol and (S)-3-amino-(S)-2-butanol O-phosphate to 2-butanone. The enzyme was also able to catalyze the conversion of both (R) and (S)-2-amino-1-propanol phosphate to propanone, with a preference for (S)-2-amino-1-propanol phosphate. The highest activity was with the proposed natural substrate DL-1-amino-2-propanol phosphate, which was converted to propionaldehyde.

The skilled person will appreciate that polypeptides having aminobutanol phosphate phospho-lyase activity isolated from a variety of sources will be useful in the present invention independent of sequence homology. One example of a suitable aminobutanol phosphate phospho-lyase enzyme is described in co-owned and co-pending US Patent Application Publication No. 20070259410A (SEQ ID NO: 126). Accordingly preferred aminobutanol phosphate phospho-lyase enzymes are those that have at least 80%-85% identity to SEQ ID NO's 126, where at least 85%-90% identity is more preferred and where at least 95% identity based on the Clustal W method of alignment using the default parameters of GAP PENALTY=10, GAP LENGTH PENALTY=0.1, and Gonnet 250 series of protein weight matrix, is most preferred.

### (f) 2-butanone to 2-butanol

This final step in all pathways to produce 2-butanol from pyruvic acid is the reduction of 2-butanone (V) to 2-butanol (VI) and is omitted when 2-butanone is the intended product. Pathway 2:

### (a) pyruvate to alpha-acetolactate

This substrate to product conversion is the same as described above for Pathway 1.

### (b) alpha-acetolactate to acetoin

This substrate to product conversion is the same as described above for Pathway 1.

### (g) acetoin to phosphoacetoin

Although enzymes that catalyze the substrate to product conversion of acetoin (II) to phosphoacetoin (VII) have not been described, the structure of the substrate acetoin is very similar to that of dihydroxyacetone, and therefore acetoin may be an acceptable substrate for dihydroxyacetone kinase (EC 2.7.1.29), an enzyme which catalyzes phosphorylation of dihydroxyacetone. Protein engineering techniques for the alteration of substrate specificity of enzymes are well known (Antikainen and Martin (2005) Bioorg. Med. Chem. 13:2701-2716) and may be used to generate an enzyme with the required specificity. In this conversion, the phosphate moiety may be supplied by any high energy biological phosphate donor, with the common substrates being phosphoenolpyruvate (as in the E. coli dihydroxyacetone kinase) and ATP (as in the Citrobacter freundii dihydroxyacetone kinase) (Garcia-Alles et al. (2004) Biochemistry 43:13037-13045).

### (h) phosphoacetoin to 3-amino-2-butanol O-phosphate

Although enzymes that catalyze the substrate to product conversion of phosphoacetoin (VII) to 3-amino-2-butanol O-phosphate (IV) have not been described, the structure of the substrate is very similar to that of dihydroxyacetone phosphate, a substrate for the proposed serinol phosphate aminotransferase encoded by the 5′ portion of the rtxA gene in some species of Bradyrhizobium (Yasuta et al., supra). Thus a serinol phosphate aminotransferase may be functional in this step.

### (e) 3-amino-2-butanol O-phosphate to 2-butanone

This substrate to product conversion is the same as described above for Pathway 1.

### (f) 2-butanone to 2-butanol

This substrate to product conversion is the same as described above for Pathway 1.

### Pathway 3:

### (a) pyruvate to alpha-acetolactate

This substrate to product conversion is the same as described above for Pathway 1.

### (b) alpha-acetolactate to acetoin

This substrate to product conversion is the same as described above for Pathway 1.

### (i) acetoin to 2,3-butanediol

The substrate to product conversion of acetoin (II) to 2,3-butanediol (VIII) may be catalyzed by a butanediol dehydrogenase that may either utilize NADH or NADPH as the source of reducing equivalents when carrying out reductions. Enzymes with activity towards acetoin participate in the pathway for production of 2,3-butanediol in organisms that produce that compound. The reported enzymes (e.g., BudC from Klebsiella pneumoniae (Ui et al. (2004) Letters in Applied Microbiology 39:533-537) generally utilize NADH. Either cofactor is acceptable for use in the production of 2-butanone by this pathway.

### (j) 2,3-butanediol to 2-butanone

The substrate to product conversion of 2,3-butanediol (VII) to 2-butanone (V) may be catalyzed by diol dehydratase enzymes (EC 4.2.1.28) and glycerol dehydratase enzymes (EC 4.2.1.30). The best characterized diol dehydratase is the coenzyme B12-dependent Klebsiella oxytoca enzyme, but similar enzymes are found in many enteric bacteria. The K. oxytoca enzyme has been shown to accept meso-2,3-butanediol as a substrate (Bachovchin et al. (1977) Biochemistry 16:1082-1092), producing the desired product 2-butanone. Applicants have identified a Klebsiella pneumoniae glycerol dehydratase which is shown to convert meso-2,3-butanediol to 2-butanone. The three subunits of the Klebsiella pneumoniae glycerol dehydratase (alpha: SEQ ID NO:145 (coding region) and 146 (protein); beta: SEQ ID NO: 147 (coding region) and 148 (protein); and gamma: SEQ ID NO: 149 (coding region) and 150 (protein)) were expressed in conjunction with the two subunits of the Klebsiella pneumoniae glycerol dehydratase reactivase (large subunit, SEQ ID NO: 151 (coding region) and 152 (protein); and small subunit, SEQ ID NO: 153 (coding region) and 154 (protein)) to provide activity.

There are also reports in the literature of a B12-independent diol dehydratase from Clostridium glycolicum (Hartmanis et al. (1986) Arch. Biochem. Biophys. 245:144-152). This enzyme has activity towards 2,3-butanediol, although this activity is less than 1% of the activity towards ethanediol, but the enzyme may be engineered to improve that activity. A better-characterized B12-independent dehydratase is the glycerol dehydratase from Clostridium butyricum (O'Brien et al. (2004) Biochemistry 43:4635-4645), which has high activity towards 1,2-propanediol as well as glycerol. This enzyme uses S-adenosylmethionine as a source of adenosyl radical. There are no reports of activity towards 2,3-butanediol, but such activity, if not already present, may possibly be engineered.

The skilled person will appreciate that polypeptides having butanediol dehydrogenase activity isolated from a variety of sources will be useful in the present invention independent of sequence homology. As noted above a variety of diol and glycerol dehydratases have been described in the literature and will be suitable for use in the present invention. Accordingly, in one aspect of the invention preferred diol and glycerol dehydratase enzymes are those that have at least 80%-85% identity to enzymes having the large, medium and small subunits, respectively of the sequences listed below:

a) SEQ ID NO:8, SEQ ID NO:10, and SEQ ID NO:12;

b) SEQ ID NO:93, SEQ ID NO:95, and SEQ ID NO:97;

c) SEQ ID NO:99, SEQ ID NO:101, and SEQ ID NO:103;

d) SEQ ID NO:105, SEQ ID NO:107, and SEQ ID NO:109;

e) SEQ ID NO:135, SEQ ID NO:136, and SEQ ID NO:137;

f) SEQ ID NO:138, SEQ ID NO:139, and SEQ ID NO:140;

g) SEQ ID NO:146, SEQ ID NO:148, and SEQ ID NO:150;

h) SEQ ID NO:141, SEQ ID NO:142, and SEQ ID NO:143; and

i) SEQ ID NO:164, SEQ ID NO:165, and SEQ ID NO:166.

where at least 85%-90% identity is more preferred and where at least 95% identity based on the Clustal W method of alignment using the default parameters of GAP PENALTY=10, GAP LENGTH PENALTY=0.1, and Gonnet 250 series of protein weight matrix, is most preferred.

Similarly preferred diol and glycerol dehydratase enzymes are those that have at least 80%-85% identity to enzymes having the large, medium and small subunits, respectively of the sequences listed below: Large subunit: SEQ ID NOs: 8, 99, 105, 135, 138, 141, 146, and 164; Medium subunit: SEQ ID NOs: 10, 101, 107, 136, 139, 142, 148, and 165; Small subunit: SEQ ID NOs:12, 103, 109, 137, 140, 143, 150, and 166; where at least 85%-90% identity is more preferred and where at least 95% identity based on the Clustal W method of alignment using the default parameters of GAP PENALTY=10, GAP LENGTH PENALTY=0.1, and Gonnet 250 series of protein weight matrix, is most preferred.

### (f) 2-butanone to 2-butanol

This substrate to product conversion is the same as described above for Pathway 1.

### Pathway 4:

### (a) pyruvate to alpha-acetolactate

This substrate to product conversion is the same as described above for Pathway 1.

### (k) alpha-acetolactate to 2,3-dihydroxy-2-methylbutanoic acid

The substrate to product conversion of acetolactate (I) to 2,3-dihydroxy-2-methylbutanoic acid (IX) is not known in the art. However, the product of this conversion has been reported as a component of fermentation broths (Ziadi et al. (1973) Comptes Rendus des Seances de l'Academie des Sciences, Serie D: Sciences Naturelles 276:965-8), but the mechanism of formation is unknown. The likely mechanism of formation is reduction of acetolactate with NADH or NADPH as the electron donor. To utilize this pathway for production of 2-butanone, an enzyme catalyzing this reaction needs to be identified or engineered. However, the precedent for enzymatic reduction of ketones to alcohols is well established.

### (l) 2,3-dihydroxy-2-methylbutanoic acid to 2-hydroxy-2-methyl-3-phosphobutanoic acid

There are no enzymes known that catalyze the substrate to product conversion of 2,3-dihydroxy-2-methylbutanoic acid (IX) to 2-hydroxy-2-methyl-3-phosphobutanoic acid (X). However, there are a large number of kinases in Nature that possess varying specificity. It is therefore likely that an enzyme could be isolated or engineered with this activity.

### (m) 2-hydroxy-2-methyl-3-phosphobutanoic acid to 2-butanone

There are no known enzymes that catalyze the substrate to product conversion of 2-hydroxy-2-methyl-3-phosphobutanoic acid (X) to 2-butanone (V). The combination of this reaction with the previous one is very similar to the multi-step reaction catalyzed by mevalonate-5-pyrophosphate (M5PP) decarboxylase, which consists of initial phosphorylation of M5PP to 3-phosphomevalonate-5-PP, followed by decarboxylation-dependent elimination of phosphate (Alvear et al. (1982) Biochemistry 21:4646-4650).

### (f) 2-butanone to 2-butanol

This substrate to product conversion is the same as described above for Pathway 1.

Thus, in providing multiple recombinant pathways from pyruvate to 2-butanone, there exists a number of choices to fulfill the individual conversion steps, and the person of skill in the art will be able to utilize publicly available sequences and sequences disclosed herein to construct the relevant pathways. A listing of a representative number of genes known in the art and useful in the construction of 2-butanone biosynthetic pathways is given above in Tables 1 and 2.

### Microbial Hosts for 2-Butanone Production

Microbial hosts for 2-butanone production may be selected from bacteria, cyanobacteria, filamentous fungi and yeasts. The microbial host used for 2-butanone production should be tolerant to the product produced, so that the yield is not limited by toxicity of the product to the host. The selection of a microbial host for 2-butanone production is described in detail below.

Microbes that are metabolically active at high titer levels of 2-butanone are not well known in the art. Although butanol-tolerant mutants have been isolated from solventogenic Clostridia, little information is available concerning the butanone tolerance of potentially useful bacterial strains. Most of the studies on the comparison of alcohol tolerance in bacteria suggest that butanol is more toxic than ethanol (de Cavalho et al., Microsc. Res. Tech. 64:215-22 (2004) and Kabelitz et al., FEMS Microbiol. Lett. 220:223-227 (2003)). Tomas et al. (J. Bacteriol. 186:2006-2018 (2004)) report that the yield of 1-butanol during fermentation in Clostridium acetobutylicum may be limited by butanol toxicity. The primary effect of 1-butanol on Clostridium acetobutylicum is disruption of membrane functions (Hermann et al., Appl. Environ. Microbiol. 50:1238-1243 (1985)).

The microbial hosts selected for the production of 2-butanone should be tolerant to 2-butanone and should be able to convert carbohydrates to 2-butanone using the introduced biosynthetic pathway. The criteria for selection of suitable microbial hosts include the following: intrinsic tolerance to 2-butanone, high rate of carbohydrate utilization, availability of genetic tools for gene manipulation, and the ability to generate stable chromosomal alterations.

Suitable host strains with a tolerance for 2-butanone may be identified by screening based on the intrinsic tolerance of the strain. The intrinsic tolerance of microbes to 2-butanone may be measured by determining the concentration of 2-butanone that is responsible for 50% inhibition of the growth rate (IC50) when grown in a minimal medium. The IC50 values may be determined using methods known in the art. For example, the microbes of interest may be grown in the presence of various amounts of 2-butanone and the growth rate monitored by measuring the optical density at 600 nanometers. The doubling time may be calculated from the logarithmic part of the growth curve and used as a measure of the growth rate. The concentration of 2-butanone that produces 50% inhibition of growth may be determined from a graph of the percent inhibition of growth versus the 2-butanone concentration. Preferably, the host strain should have an IC50 for 2-butanone of greater than about 0.5%. More suitable is a host strain with an IC50 for 2-butanone that is greater than about 1.5%. Particularly suitable is a host strain with an IC50 for 2-butanone that is greater than about 2.5%.

The microbial host for 2-butanone production should also utilize glucose and/or other carbohydrates at a high rate. Most microbes are capable of utilizing carbohydrates. However, certain environmental microbes cannot efficiently use carbohydrates, and therefore would not be suitable hosts.

The ability to genetically modify the host is essential for the production of any recombinant microorganism. Modes of gene transfer technology that may be used include by electroporation, conjugation, transduction or natural transformation. A broad range of host conjugative plasmids and drug resistance markers are available. The cloning vectors used with an organism are tailored to the host organism based on the nature of antibiotic resistance markers that can function in that host.

The microbial host also may be manipulated in order to inactivate competing pathways for carbon flow by inactivating various genes. This requires the availability of either transposons or chromosomal integration vectors to direct inactivation. Additionally, production hosts that are amenable to chemical mutagenesis may undergo improvements in intrinsic 2-butanone tolerance through chemical mutagenesis and mutant screening.

Based on the criteria described above, suitable microbial hosts for the production of 2-butanone include, but are not limited to, members of the genera Clostridium, Zymomonas, Escherichia, Salmonella, Rhodococcus, Pseudomonas, Bacillus, Lactobacillus, Enterococcus, Pediococcus, Alcaligenes, Klebsiella, Paenibacillus, Arthrobacter, Corynebacterium, Brevibacterium, Pichia, Candida, Hansenula and Saccharomyces. Preferred hosts include: Escherichia coli, Alcaligenes eutrophus, Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Pediococcus pentosaceus, Pediococcus acidilactici, Bacillus subtilis and Saccharomyces cerevisiae.

### Construction of Production Host

Recombinant organisms containing the necessary genes that encode the enzymatic pathway for the conversion of a fermentable carbon substrate to 2-butanone may be constructed using techniques well known in the art. Genes encoding the enzymes of, for example, the 2-butanone biosynthetic Pathway 1: acetolactate synthase, acetolactate decarboxylase, acetoin aminase (or amine:pyruvate transaminase), aminobutanol kinase, and aminobutanol O-phosphate lyase may be isolated from various sources, as described above.

Methods of obtaining desired genes from a bacterial genome are common and well known in the art of molecular biology. For example, if the sequence of the gene is known, primers may be designed and the desired sequence amplified using standard primer-directed amplification methods such as polymerase chain reaction (U.S. Pat. No. 4,683,202) to obtain amounts of DNA suitable for cloning into expression vectors. If a gene that is heterologous to a known sequence is to be isolated, suitable genomic libraries may be created by restriction endonuclease digestion and may be screened with probes having complementary sequence to the desired gene sequence. Once the sequence is isolated, the DNA may be amplified using standard primer-directed amplification methods such as polymerase chain reaction (U.S. Pat. No. 4,683,202) to obtain amounts of DNA suitable for cloning into expression vectors, which are then transformed into appropriate host cells.

In addition, given the amino acid sequence of a protein with desired enzymatic activity, the coding sequence may be ascertained by reverse translating the protein sequence. A DNA fragment containing the coding sequence may be prepared synthetically and cloned into an expression vector, then transformed into the desired host cell.

In preparing a synthetic DNA fragment containing a coding sequence, this sequence may be optimized for expression in the target host cell. Tools for codon optimization for expression in a heterologous host are readily available. Some tools for codon optimization are available based on the GC content of the host organism. The GC contents of some exemplary microbial hosts are given Table 3.

TABLE 3
GC Contents of Microbial Hosts

| Strain | % GC |
|---|---|
| B. licheniformis | 46 |
| B. subtilis | 42 |
| C. acetobutylicum | 37 |
| E. coli | 50 |
| P. putida | 61 |
| A. eutrophus | 61 |
| Paenibacillus macerans | 51 |
| Rhodococcus erythropolis | 62 |
| Brevibacillus | 50 |
| Paenibacillus polymyxa | 50 |

Once the relevant pathway genes are identified and isolated they may be transformed into suitable expression hosts by means well known in the art. Vectors useful for the transformation of a variety of host cells are common and commercially available from companies such as EPICENTRE® (Madison, Wis.), Invitrogen Corp. (Carlsbad, Calif.), Stratagene (La Jolla, Calif.), and New England Biolabs, Inc. (Beverly, Mass.). Typically the vector contains a selectable marker and sequences allowing autonomous replication or chromosomal integration in the desired host. In addition, suitable vectors comprise a promoter region which harbors transcriptional initiation controls and a transcriptional termination control region, between which a coding region DNA fragment may be inserted, to provide expression of the inserted coding region. Both control regions may be derived from genes homologous to the transformed host cell, although it is to be understood that such control regions may also be derived from genes that are not native to the specific species chosen as a production host.

Initiation control regions or promoters, which are useful to drive expression of the relevant pathway coding regions in the desired host cell are numerous and familiar to those skilled in the art. Virtually any promoter capable of driving these genetic elements is suitable for use including, but not limited to, promoters derived from the following genes: CYC1, HIS3, GAL1, GAL10, ADH1, PGK, PHO5, GAPDH, ADC1, TRP1, URA3, LEU2, ENO, TPI, CUP1, FBA, GPD, and GPM (useful for expression in Saccharomyces); AOX1 (useful for expression in Pichia); as well as the lac, ara, tet, trp, IP_{L}, IP_{R}, T7, tac, and trc promoters (useful for expression in Escherichia coli, Alcaligenes, and Pseudomonas); the amy, apr, and npr promoters, and various phage promoters useful for expression in Bacillus subtilis, Bacillus licheniformis, and Paenibacillus macerans; nisA (useful for expression Gram-positive bacteria, Eichenbaum et al. Appl. Environ. Microbiol. 64(8):2763-2769 (1998)); and the synthetic P11 promoter (useful for expression in Lactobacillus plantarum, Rud et al., Microbiology 152:1011-1019 (2006)).

Termination control regions may also be derived from various genes native to the preferred hosts. Optionally, a termination site may be unnecessary, however, it is most preferred if included.

Certain vectors are capable of replicating in a broad range of host bacteria and can be transferred by conjugation. The complete and annotated sequence of pRK404 and three related vectors: pRK437, pRK442, and pRK442(H), are available. These derivatives have proven to be valuable tools for genetic manipulation in Gram-negative bacteria (Scott et al., Plasmid 50(1):74-79 (2003)). Several plasmid derivatives of broad-host-range Inc P4 plasmid RSF1010 are also available with promoters that can function in a range of Gram-negative bacteria. Plasmid pAYC36 and pAYC37, have active promoters along with multiple cloning sites to allow for heterologous gene expression in Gram-negative bacteria.

Chromosomal gene replacement tools are also widely available. For example, a thermosensitive variant of the broad-host-range replicon pWV101 has been modified to construct a plasmid pVE6002 which can be used to effect gene replacement in a range of Gram-positive bacteria (Maguin et al., J. Bacteriol. 174(17):5633-5638 (1992)).

The expression of a 2-butanone biosynthetic pathway in various preferred microbial hosts is described in more detail below.

### Expression of a 2-Butanone Biosynthetic Pathway in E. coli

Vectors useful for the transformation of E. coli are common and commercially available from the companies listed above. For example, the genes of a 2-butanone biosynthetic pathway may be isolated from various sources, as described above, cloned onto a modified pUC19 vector and transformed into E. coli NM522, as described in Examples 10 and 11. Alternatively, the genes encoding a 2-butanone biosynthetic pathway may be divided into multiple operons, cloned onto expression vectors, and transformed into various E. coli strains, as described in Examples 13, 14, and 15.

### Expression of a 2-Butanone Biosynthetic Pathway in Rhodococcus erythropolis

A series of E. coli-Rhodococcus shuttle vectors are available for expression in R. erythropolis, including, but not limited to pRhBR17 and pDA71 (Kostichka et al., Appl. Microbiol. Biotechnol. 62:61-68 (2003)). Additionally, a series of promoters are available for heterologous gene expression in R. erythropolis (see for example Nakashima et al., Appl. Environ. Microbiol. 70:5557-5568 (2004), and Tao et al., Appl. Microbiol. Biotechnol. 2005, DOI 10.1007/s00253-005-0064). Targeted gene disruptions in chromosomal genes of R. erythropolis may be created using the methods described by Tao et al., supra, and Brans et al. (Appl. Envion. Microbiol. 66: 2029-2036 (2000)).

The heterologous genes required for the production of 2-butanone, as described above, may be cloned initially in pDA71 or pRhBR71 and transformed into E. coli. The vectors may then be transformed into R. erythropolis by electroporation, as described by Kostichka et al., supra. The recombinants may be grown in synthetic medium containing glucose and the production of 2-butanone can be followed using fermentation methods known in the art.

### Expression of a 2-Butanone Biosynthetic Pathway in B. Subtilis

Methods for gene expression and creation of mutations in B. subtilis are also well known in the art. For example, the genes of a 2-butanone biosynthetic pathway may be isolated from various sources, as described above, cloned into a modified E. coli-Bacillus shuttle vector and transformed into Bacillus subtilis BE1010, as described in Example 12, The desired genes may be cloned into a Bacillus expression vector and transformed into a strain to make a production host. Alternatively, the genes may be integrated into the Bacillus chromosome using conditional replicons or suicide vectors that are known to one skilled in the art. For example, the Bacillus Genetic Stock Center carries numerous integration vectors.

### Expression of a 2-Butanone Biosynthetic Pathway in B. licheniformis

Most of the plasmids and shuttle vectors that replicate in B. subtilis may be used to transform B. licheniformis by either protoplast transformation or electroporation. The genes required for the production of 2-butanone may be cloned in plasmids pBE20 or pBE60 derivatives (Nagarajan et al., Gene 114:121-126 (1992)). Methods to transform B. licheniformis are known in the art (for example see Fleming et al. Appl. Environ. Microbiol., 61(11):3775-3780 (1995)). The plasmids constructed for expression in B. subtilis may be transformed into B. licheniformis to produce a recombinant microbial host that produces 2-butanone.

### Expression of a 2-Butanone Biosynthetic Pathway in Paenibacillus macerans

Plasmids may be constructed as described above for expression in B. subtilis and used to transform Paenibacillus macerans by protoplast transformation to produce a recombinant microbial host that produces 2-butanone.

### Expression of a 2-Butanone Biosynthetic Pathway in Alcaligenes (Ralstonia) eutrophus

Methods for gene expression and creation of mutations in Alcaligenes eutrophus are known in the art (see for example Taghavi et al., Appl. Environ. Microbiol., 60(10):3585-3591 (1994)). The genes for a 2-butanone biosynthetic pathway may be cloned in any of the broad host range vectors described above, and electroporated into Alcaligenes eutrophus to generate recombinants that produce 2-butanone. The poly(hydroxybutyrate) pathway in Alcaligenes has been described in detail, a variety of genetic techniques to modify the Alcaligenes eutrophus genome are known, and those tools can be applied for engineering a 2-butanone biosynthetic pathway.

### Expression of a 2-Butanone Biosynthetic Pathway in Pseudomonas putida

Methods for gene expression in Pseudomonas putida are known in the art (see for example Ben-Bassat et al., U.S. Pat. No. 6,586,229, which is incorporated herein by reference). The genes of a 2-butanone biosynthetic pathway may be inserted into pPCU18, and this ligated DNA may be electroporated into electrocompetent Pseudomonas putida DOT-T1 C5aAR1 cells to generate recombinants that produce 2-butanone.

### Expression of a 2-butanone biosynthetic pathway in Lactobacillus plantarum

The Lactobacillus genus belongs to the Lactobacillales family and many plasmids and vectors used in the transformation of Bacillus subtilis and Streptococcus may be used for Lactobacillus. Non-limiting examples of suitable vectors include pAMβ1 and derivatives thereof (Renault et al., Gene 183:175-182 (1996); and O'Sullivan et al., Gene 137:227-231 (1993)); pMBB1 and pHW800, a derivative of pMBB1 (Wyckoff et al. Appl Environ. Microbiol. 62:1481-1486 (1996)); pMG1, a conjugative plasmid (Tanimoto et al., J. Bacteriol. 184:5800-5804 (2002)); pNZ9520 (Kleerebezem et al., Appl. Environ. Microbiol. 63:4581-4584 (1997)); pAM401 (Fujimoto et al., Appl. Environ. Microbiol. 67:1262-1267 (2001)); and pAT392 (Arthur et al., Antimicrob. Agents Chemother. 38:1899-1903 (1994)). Several plasmids from Lactobacillus plantarum have also been reported (van Kranenburg et al., Appl. Environ. Microbiol. 71(3):1223-1230 (2005)).

The various genes for a 2-butanone biosynthetic pathway may be assembled into any suitable vector, such as those described above. The codons can be optimized for expression based on the codon index deduced from the genome sequences of Lactobacillus plantarum or Lactobacillus arizonensis. The plasmids may be introduced into the host cell using methods known in the art, such as electroporation (Cruz-Rodz et al. Molecular Genetics and Genomics 224:1252-154 (1990), Bringel, et al. Appl. Microbiol. Biotechnol. 33: 664-670 (1990), Alegre et al., FEMS Microbiology letters 241:73-77 (2004)), and conjugation (Shrago et al., Appl. Environ. Microbiol. 52:574-576 (1986)). The 2-butanone biosynthetic pathway genes can also be integrated into the chromosome of Lactobacillus using integration vectors (Hols et al., Appl. Environ. Microbiol. 60:1401-1403 (1990), Jang et al., Micro. Lett. 24:191-195 (2003)).

### Expression of a 2-Butanone Biosynthetic Pathway in Enterococcus faecium, Enterococcus gallinarium, and Enterococcus faecalis

The Enterococcus genus belongs to the Lactobacillales family and many plasmids and vectors used in the transformation of Lactobacillus, Bacillus subtilis, and Streptococcus, described above, may be used for Enterococcus. Expression vectors for E. faecalis using the nisA gene from Lactococcus may also be used (Eichenbaum et al., Appl. Environ. Microbiol. 64:2763-2769 (1998). Additionally, vectors for gene replacement in the E. faecium chromosome may be used (Nallaapareddy et al., Appl. Environ. Microbiol. 72:334-345 (2006)).

The various genes for a 2-butanone biosynthetic pathway may be assembled into any suitable vector, such as those described above. The codons can be optimized for expression based on the codon index deduced from the genome sequences of Enterococcus faecalis or Enterococcus faecium. The plasmids may be introduced into the host cell using methods known in the art, such as electroporation, as described by Cruz-Rodz et al. (Molecular Genetics and Genomics 224:1252-154 (1990)) or conjugation, as described by Tanimoto et al. (J. Bacteriol. 184:5800-5804 (2002)) and Grohamann et al. (Microbiol. Mol. Biol. Rev. 67:277-301 (2003)).

### Expression of a 2-Butanone Biosynthetic Pathway in Pediococcus pentosaceus and Pediococcus acidilactici

The Pediococcus genus belongs to the Lactobacillales family and many plasmids and vectors used in the transformation of Bacillus subtilis and Streptococcus, described above, may be used for Pediococcus. A non-limiting example of a suitable vector is pHPS9 (Bukhtiyarova et al. Appl. Environ. Microbiol. 60:3405-3408 (1994)). Several plasmids from Pediococcus have also been reported (Alegre et al., FEMS Microbiol. Lett. 250:151-156 (2005); Shareck et al. Crit. Rev Biotechnol. 24:155-208 (2004)).

The genes for a 2-butanone biosynthetic pathway may be assembled into any suitable vector, such as those described above. The codons can be optimized for expression based on the codon index deduced from the genome sequence of Pediococcus pentosaceus. The plasmids may be introduced into the host cell using methods known in the art, such as electroporation (see for example, Osmanagaoglu et al., J. Basic Microbiol. 40:233-241 (2000); Alegre et al., FEMS Microbiol. Lett. 250:151-156 (2005)) and conjugation (Gonzalez and Kunka, Appl. Environ. Microbiol. 46:81-89 (1983)). The 2-butanone biosynthetic pathway genes can also be integrated into the chromosome of Pediococcus using integration vectors (Davidson et al. Antonie van Leeuwenhoek 70:161-183 (1996)).

### Fermentation Media

Fermentation media in the present invention must contain suitable carbon substrates. Suitable substrates may include but are not limited to monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt. Additionally the carbon substrate may also be one-carbon substrates such as carbon dioxide, or methanol for which metabolic conversion into key biochemical intermediates has been demonstrated. In addition to one and two carbon substrates, methylotrophic organisms are also known to utilize a number of other carbon containing compounds such as methylamine, glucosamine and a variety of amino acids for metabolic activity. For example, methylotrophic yeasts are known to utilize the carbon from methylamine to form trehalose or glycerol (Bellion et al., Microb. Growth C1-Compd., [Int. Symp.], 7th (1993), 415-32, Editor(s): Murrell, J. Collin; Kelly, Don P. Publisher: Intercept, Andover, UK). Similarly, various species of Candida will metabolize alanine or oleic acid (Sulter et al., Arch. Microbiol. 153:485-489 (1990)). Hence it is contemplated that the source of carbon utilized in the present invention may encompass a wide variety of carbon containing substrates and will only be limited by the choice of organism.

Although it is contemplated that all of the above mentioned carbon substrates and mixtures thereof are suitable in the present invention, preferred carbon substrates are glucose, fructose, and sucrose. Sucrose may be derived from renewable sugar sources such as sugar cane, sugar beets, cassava, sweet sorghum, and mixtures thereof. Glucose and dextrose may be derived from renewable grain sources through saccharification of starch based feedstocks including grains such as corn, wheat, rye, barley, oats, and mixtures thereof. In addition, fermentable sugars may be derived from renewable cellulosic or lignocellulosic biomass through processes of pretreatment and saccharification, as described, for example, in co-owned and co-pending U.S. Patent Application Publication No. 2007/0031918A1, which is herein incorporated by reference. Biomass refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass may comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, animal manure, and mixtures thereof.

In addition to an appropriate carbon source, fermentation media must contain suitable minerals, salts, cofactors, buffers and other components, known to those skilled in the art, suitable for the growth of the cultures and promotion of an enzymatic pathway necessary for 2-butanone production.

Culture Conditions with Temperature Lowering

In the present method, the recombinant microbial production host which produces 2-butanone is seeded into a fermentation medium comprising a fermentable carbon substrate to create a fermentation culture. The production host is grown in the fermentation culture at a first temperature for a first period of time. The first temperature is typically from about 25° C. to about 40° C.

Suitable fermentation media in the present invention include common commercially prepared media such as Luria Bertani (LB) broth, Sabouraud Dextrose (SD) broth or Yeast Medium (YM) broth. Other defined or synthetic growth media may also be used, and the appropriate medium for growth of the particular microorganism will be known by one skilled in the art of microbiology or fermentation science. The use of agents known to modulate catabolite repression directly or indirectly, e.g., cyclic adenosine 2′:3′-monophosphate, may also be incorporated into the fermentation medium.

Suitable pH ranges for the fermentation are between pH 5.0 to pH 9.0, where pH 6.0 to pH 8.0 is preferred as the initial condition.

Fermentations may be performed under aerobic or anaerobic conditions, where anaerobic or microaerobic conditions are preferred.

The first period of time to grow the production host at the first temperature may be determined in a variety of ways. For example, during this period of growth a metabolic parameter of the fermentation culture may be monitored. The metabolic parameter that is monitored may be any parameter known in the art, including, but not limited to the optical density, pH, respiratory quotient, fermentable carbon substrate utilization, CO₂ production, and 2-butanone production. During this period of growth, additional fermentable carbon substrate may be added, the pH may be adjusted, oxygen may be added for aerobic cells, or other culture parameters may be adjusted to support the metabolic activity of the culture. Though nutrients and culture conditions are supportive of growth, after a period of time the metabolic activity of the fermentation culture decreases as determined by the monitored parameter described above. For example, a decrease in metabolic activity may be indicated by a decrease in one or more of the following parameters: rate of optical density change, rate of pH change, rate of change in respiratory quotient (if the host cells are aerobic), rate of fermentable carbon substrate utilization, rate of 2-butanone production, rate of change in CO₂ production, or rate of another metabolic parameter. The decrease in metabolic activity is related to the sensitivity of the host cells to the production of 2-butanone and/or the presence of 2-butanone in the culture. When decreased metabolic activity is detected, the temperature of the fermentation culture is lowered to reduce the sensitivity of the host cells to 2-butanonel and thereby allow further production of 2-butanone. In one embodiment, the lowering of the temperature coincides with a change in the metabolic parameter that is monitored.

In one embodiment, the change in metabolic activity is a decrease in the rate of 2-butanone production. 2-Butanone production may be monitored by analyzing the amount of 2-butanone present in the fermentation culture medium as a function of time using methods well known in the art, such as using high performance liquid chromatography (HPLC) or gas chromatography (GC), which are described in the Examples herein. GC is preferred due to the short assay time.

Alternatively, the lowering of the temperature of the fermentation culture may occur at a predetermined time. The first period of time may be predetermined by establishing a correlation between a metabolic parameter of the fermentation culture and time in a series of test fermentations runs. A correlation between a metabolic parameter, as described above, and time of culture growth may be established for any 2-butanone producing host by one skilled in the art. The specific correlation may vary depending on conditions used including, but not limited to, carbon substrate, fermentation conditions, and the specific recombinant 2-butanone producing microbial production host. The correlation is most suitably made between 2-butanone production or specific glucose consumption rate and time of culture growth. Once the predetermined time has been established from the correlation, the temperature of the fermentation culture in subsequent fermentation runs is lowered at the predetermined time. For example, if it is determined by monitoring a metabolic parameter in the test fermentation runs that the rate of production of 2-butanone decreases after 12 hours, the temperature in subsequent fermentations runs is lowered after 12 hours without the need to monitor 2-butanone production in the subsequent runs.

After the first period of time, the temperature of the fermentation culture is lowered to a second temperature. Typically, the second temperature is about 3° C. to about 25° C. lower than the first temperature. Reduction in temperature to enhance tolerance of the host cells to 2-butanone is balanced with maintaining the temperature at a level where the cells continue to be metabolically active for 2-butanone production. For example, a fermentation culture that has been grown at about 35° C. may be reduced in temperature to about 28° C.; or a culture grown at about 30° C. may be reduced in temperature to about 25° C. The change in temperature may be done gradually over time or may be made as a step change. The production host is incubated at the second temperature for a second period of time, so that 2-butanone production continues. The second period of time may be determined in the same manner as the first period of time described above, e.g., by monitoring a metabolic parameter or by using a predetermined time.

Additionally, the temperature lowering and incubation steps may be repeated one or more times to more finely balance metabolic activity for 2-butanone production and 2-butanone sensitivity. For example, a culture that has been grown at about 35° C. may be reduced in temperature to about 32° C., followed by an incubation period. During this period a metabolic parameter of the fermentation culture may be monitored as described above, or a predetermined time may be used. It is particularly suitable to monitor the production of 2-butanone during this incubation period. When monitoring indicates a decrease in metabolic activity or at a predetermined time, the temperature may be reduced a second time. For example, the temperature may be reduced from about 32° C. to about 28° C. The temperature lowering and incubation steps may be repeated a third time where the temperature is reduced, for example, to about 20° C. The production host is incubated at the lowered temperature so that 2-butanone production continues. The steps may be repeated further as necessary to obtain the desired 2-butanone titer.

### Industrial Batch and Continuous Fermentations

The present process employs a batch method of fermentation. A classical batch fermentation is a closed system where the composition of the medium is set at the beginning of the fermentation and not subject to artificial alterations during the fermentation. Thus, at the beginning of the fermentation the medium is inoculated with the desired organism or organisms, and fermentation is permitted to occur without adding anything to the system. Typically, however, a “batch” fermentation is batch with respect to the addition of carbon source and attempts are often made at controlling factors such as pH and oxygen concentration. In batch systems the metabolite and biomass compositions of the system change constantly up to the time the fermentation is stopped. Within batch cultures cells moderate through a static lag phase to a high growth log phase and finally to a stationary phase where growth rate is diminished or halted. If untreated, cells in the stationary phase will eventually die. Cells in log phase generally are responsible for the bulk of production of end product or intermediate.

A variation on the standard batch system is the fed-batch system. Fed-batch fermentation processes are also suitable in the present invention and comprise a typical batch system with the exception that the substrate is added in increments as the fermentation progresses. Fed-batch systems are useful when catabolite repression is apt to inhibit the metabolism of the cells and where it is desirable to have limited amounts of substrate in the media. Measurement of the actual substrate concentration in fed-batch systems is difficult and is therefore estimated on the basis of the changes of measurable factors such as pH, dissolved oxygen and the partial pressure of waste gases such as CO₂. Batch and fed-batch fermentations are common and well known in the art and examples may be found in Thomas D. Brock in Biotechnology: A Textbook of Industrial Microbiology, Second Edition (1989) Sinauer Associates, Inc., Sunderland, M A., or Deshpande, Mukund V., Appl. Biochem. Biotechnol., 36:227, (1992), herein incorporated by reference.

Although the present invention is performed in batch mode it is contemplated that the method would be adaptable to continuous fermentation methods. Continuous fermentation is an open system where a defined fermentation medium is added continuously to a bioreactor and an equal amount of conditioned media is removed simultaneously for processing. Continuous fermentation generally maintains the cultures at a constant high density.

Continuous fermentation allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by the turbidity of the culture medium, is kept constant. Continuous systems strive to maintain steady state growth conditions and thus the cell loss due to the medium being drawn off must be balanced against the cell growth rate in the fermentation. Methods of modulating nutrients and growth factors for continuous fermentation processes as well as techniques for maximizing the rate of product formation are well known in the art of industrial microbiology and a variety of methods are detailed by Brock, supra.

It is contemplated that the present invention may be practiced using either batch, fed-batch or continuous processes and that any known mode of fermentation would be suitable. Additionally, it is contemplated that cells may be immobilized on a substrate as whole cell catalysts and subjected to fermentation conditions for 2-butanone production.

Methods for 2-Butanone Isolation from the Fermentation Medium

The bioproduced 2-butanone may be isolated from the fermentation medium using methods known in the art for ABE fermentations (see for example, Durre, Appl. Microbiol. Biotechnol. 49:639-648 (1998), Groot et al., Process Biochem. 27:61-75 (1992), and references therein). For example, solids may be removed from the fermentation medium by centrifugation, filtration, decantation, or the like. Then, the 2-butanone may be isolated from the fermentation medium using methods such as distillation, azeotropic distillation, liquid-liquid extraction, adsorption, gas stripping, membrane evaporation, or pervaporation.

### EXAMPLES

The present invention is further defined in the following Examples. It should be understood that these Examples, while indicating a preferred embodiment of the invention, are given by way of illustration only. From the above discussion and these Examples, one skilled in the art can ascertain the essential characteristics of this invention, and, without departing from the spirit and scope thereof, can make various changes and modifications of the invention to adapt it to various uses and conditions.

### General Methods

Standard recombinant DNA and molecular cloning techniques described in the Examples are well known in the art and are described by Sambrook, J., Fritsch, E. F. and Maniatis, T. Molecular Cloning: A Laboratory Manual; Cold Spring Harbor Laboratory Press: Cold Spring Harbor, N.Y., (1989) (Maniatis) and by T. J. Silhavy, M. L. Bennan, and L. W. Enquist, Experiments with Gene Fusions, Cold Spring Harbor Laboratory Press, Cold Spring Harbor, N.Y. (1984) and by Ausubel, F. M. et al., Current Protocols in Molecular Biology, pub. by Greene Publishing Assoc. and Wiley-Interscience (1987).

Materials and methods suitable for the maintenance and growth of bacterial cultures are well known in the art. Techniques suitable for use in the following Examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994)) or by Thomas D. Brock in Biotechnology: A Textbook of Industrial Microbiology, Second Edition, Sinauer Associates, Inc., Sunderland, Mass. (1989). All reagents, restriction enzymes and materials described for the growth and maintenance of bacterial cells were obtained from Aldrich Chemicals (Milwaukee, Wis.), BD Diagnostic Systems (Sparks, Md.), Life Technologies (Rockville, Md.), or Sigma Chemical Company (St. Louis, Mo.) unless otherwise specified. Bacterial strains are obtained from the American Type Culture Collection (ATCC, Manassas, Va.) unless otherwise noted.

### Oligonucleotide Primers Described in the Following Examples are Given in Table 4. All Oligonucleotide Primers were Synthesized by Sigma-Genosys (Woodlands, Tex.).

TABLE 4

Cloning and Screening Primers

| | | | | |
|---|---|---|---|---|
| | | | SEQ | |
| | Primer | | ID | |
| Gene | Name | Sequence | NO: | Description |

| | | | | |
|---|---|---|---|---|
| budB | B1 | CACCATGGACAAACAGTA | 15 | budB |
| | | TCCGGTACGCC | | forward |
| budB | B2 | CGAAGGGCGATAGCTTTA | 16 | budB |
| | | CCAATCC | | reverse |
| budA | B3 | CACCATGAATCATTCTGC | 17 | budA |
| | | TGAATGCACCTGCG | | forward |
| budA | B4 | GATACTGTTTGTCCATGT | 18 | budA |
| | | GACC | | reverse |
| budC | B5 | CACCATGAAAAAAGTCGC | 19 | budC |
| | | ACTTGTTACC | | forward |
| budC | B6 | TTAGTTAAATACCAT | 20 | budC |
| | | | | reverse |
| pddA | B7 | CACCATGAGATCGA | 21 | pddABC |
| | | AAAGATTTG | | forward |
| pddC | B8 | CTTAGAGAAGTTAATCGT | 22 | pddABC |
| | | CGCC | | reverse |
| sadh | B9 | CACCATGAAAGCCCTCCA | 23 | sadh |
| | | GTACACC | | forward |
| sadh | B10 | CGTCGTGTCATGCCCGG | 24 | sadh |
| | | G | | reverse |
| budA | B11 | GATCGAATTCGTTTAAACT | 25 | budABC |
| | | TAGTTTTCTACCGCACG | | forward |
| budC | B12 | GATCGCATGCAAGCTTTC | 26 | budABC |
| | | ATATAGTCGGAATTCC | | reverse |
| pddA | B13 | GATCGAATTCGTTTAAACA | 27 | pddABC |
| | | AAGGAGGTCTGATTCATG | | forward |
| | | AGATCG | | |
| pddC | B14 | GATCGGATTCTTAATCGT | 28 | pddABC |
| | | CGCC | | reverse |
| sadh | B15 | GATCGGATCCAAAGGAGG | 29 | sadh |
| | | TCGGGCGCATGAAAGCC | | forward |
| | | C | | |
| sadh | B16 | GATCTCTAGAAAGCTTTC | 30 | sadh |
| | | AGCCCGGGACGACC | | reverse |
| — | BenF | ACTTTCTTTCGCCTGTTTC | 31 | — |
| | | AC | | |
| — | BenBPR | CATGAAGCTTGTTTAAACT | 32 | — |
| | | CGGTGACCTTGAAAATAA | | |
| | | TGAAAACTTATATTGTTTT | | |
| | | GAAAATAATGAAAACTTAT | | |
| | | ATTG | | |
| budAB | BABC F | GAGCTCGAATTCAAAGGA | 33 | budAB |
| | | GGAAGTGTATATGAATCA | | forward |
| | | TTC | | |
| budAB | BAB R | GGATCCTCTAGAATTAGT | 34 | budAB |
| | | TAAATACCATCCCGCCG | | reverse |
| budC | BC Spe | ACTAGTAAAGGAGGAAAG | 40 | budC |
| | F | AGTATGAAGAAGGTCGCA | | forward |
| | | CT | | |
| budC | BC Xba | TCTAGAAAGCAGGGGCAA | 41 | budC |
| | R | GCCATGTC | | reverse |
| pddAB | DDo | AAGCTTAAAGGAGGCTGA | 44 | pddABC- |
| C- | For | TTCATGAGATCGAAAAGA | | ddrAB |
| ddrAB | | TT | | forward |
| pddAB | DDo | TCTAGATTATTCATCCTGC | 45 | pddABC- |
| C- | Rev | TGTTCTCC | | ddrAB |
| ddrAB | | | | reverse |
| chnA | ChnA F | CATCAATTGACTACGTAG | 54 | chnA |
| | | TCGTACGTGTAAGGAGGT | | forward |
| | | TTGAAATGGAAAAAATTAT | | |
| | | G | | |
| chnA | ChnA R | CATGCTAGCCCCGGGTAT | 55 | chnA |
| | | CTTCTACTCATTTTTTATTT | | reverse |
| | | CG | | |
| — | Top | CTAGAAGTCAAAAGCCTC | 58 | forward |
| | ter F1 | CGACCGGAGGCTTTTGA | | |
| — | Top | CTGCTCGAGTTGCTAGC | 59 | forward |
| | ter F2 | AAGTTTAAACAAAAAAAA | | |
| | | GCCCGCTCATTAGGCGG | | |
| | | GCTGAGCT | | |
| — | Bot | CAGCCCGCCTAATGAGC | 60 | reverse |
| | ter R1 | GGGCTTTTTTTTGTTTAA | | |
| | | AC | | |
| — | Bot | TTGCTAGCAACTCGAGCA | 61 | reverse |
| | ter R2 | GTCAAAAGCCTCCGGTC | | |
| | | GGAGGCTTTTGACTT | | |
| KA-AT | OT872 | CTCCGGAATTCATGTCTG | 127 | Aminoalco- |
| | | ACGGACGACTCACCGCA | | hol kinase/ |
| | | | | lyase oper- |
| | | | | on forward |
| KA-AT | OT873 | TTCCAATGCATTGGCTGC | 128 | Aminoalco- |
| | | AGTTATCTCTGTGCACGA | | hol kinase/ |
| | | GTGCCGATGA | | lyase oper- |
| | | | | on reverse |
| KA | OT879 | AACAGCCAAGCTTGGCT | 129 | Aminoalco- |
| | | GCAGTCATCGCGCATTCT | | hol kinase |
| | | CCGGG | | reverse |
| AT | OT880 | TCTCCGGAATTCATGACG | 130 | Aminoalco- |
| | | TCTGAAATGACAGCGACA | | hol lyase |
| | | GAAG | | forward |
| pBAD. | OT909 | GCTAACAGGAGGAAGAA | 131 | Adds EcoRI |
| HisB | | TTCATGGGGGGTTCTC | | site to |
| | | | | replace |
| | | | | NcoI site |
| pBAD. | OT910 | GAGAACCCCCCATGAATT | 132 | Adds EcoRI |
| HisB | | CTTCCTCCTGTTAGC | | site to |
| | | | | replace |
| | | | | NcoI site |
| BudAB | N84seqR3 | GGACCTGCTTCGCTTTAT | 159 | reverse |
| | | CG | | |
| APT | APTfor | GCGCGCCCGGGAAGAAG | 162 | APT forward |
| | | GAGCTCTTCACCATGAAC | | |
| | | AAACCACAGTCTTGG | | |
| APT | APTrev | GCGCGCCCGGGTTCATG | 163 | APT reverse |
| | | CCACCTCTGCG | | |

TABLE 5

Sequencing Primers

| | | | |
|---|---|---|---|
| | | | SEQ |
| | | Gene- | ID |
| Name | Sequence | specific | NO: |

| | | | |
|---|---|---|---|
| M13 Forward | GTAAAACGACGGCCAGT | — | 35 |
| M13 Reverse | AACAGCTATGACCATG | — | 36 |
| N83 SeqF2 | GCTGGATTACCAGCTCGACC | — | 37 |
| N83 SeqF3 | CGGACGCATTACCGGCAAAG | — | 38 |
| N84 SeqR2 | GCATCGAGATTATCGGGATG | — | 65 |
| N84 SeqR4 | CGAAGCGAGAGAAGTTATCC | — | 39 |
| Trc F | TTGACAATTAATCATCCGGC | all | 42 |
| Trc R | CTTCTCTCATCCGCCAAAAC | all | 43 |
| DDko seq F2 | GCATGGCGCGGATTTGACGAAC | pddABC- | 46 |
| | | ddrAB | |
| DDko seq F5 | CATTAAAGAGACCAAGTACGTG | pddABC- | 47 |
| | | ddrAB | |
| DDko seq F7 | ATATCCTGGTGGTGTCGTCGGCGT | pddABC- | 48 |
| | | ddrAB | |
| DDko seq F9 | TCTTTGTCACCAACGCCCTGCG | pddABC- | 49 |
| | | ddrAB | |
| DDko seq R1 | GCCCACCGCGCTCGCCGCCGCG | pddABC- | 50 |
| | | ddrAB | |
| DDko seq R3 | CCCCCAGGATGGCGGCTTCGGC | pddABC- | 51 |
| | | ddrAB | |
| DDko seq R7 | GGGCCGACGGCGATAATCACTT | pddABC- | 52 |
| | | ddrAB | |
| DDko seq R10 | TTCTTCGATCCACTCCTTAACG | pddABC- | 53 |
| | | ddrAB | |
| chnSeq F1 | CTCAACAGGGTGTAAGTGTAGT | chnA | 56 |
| chnSeq R1 | CGTTTTGATATAGCCAGGATGT | chnA | 57 |
| pCL1925 vec F | CGGTATCATCAACAGGCTTACC | all | 62 |
| pCL1925 vec R1 | AGGGTTTTCCCAGTCACGACGT | all | 63 |
| pCL1925 vec R2 | CGCAATAGTTGGCGAAGTAATC | all | 64 |
| APTseqRev | GCTAGAGATGATAGC | APT | 160 |
| APTseqFor | GGAAGAGACTATCCAGCG | APT | 161 |

### Methods for Determining 2-Butanol and 2-Butanone Concentration in Culture Media

The concentration of 2-butanol and 2-butanone in the culture media can be determined by a number of methods known in the art. For example, a specific high performance liquid chromatography (HPLC) method utilized a Shodex SH-1011 column with a Shodex SH-G guard column, both purchased from Waters Corporation (Milford, Mass.), with refractive index (RI) detection. Chromatographic separation was achieved using 0.01 M H₂SO₄ as the mobile phase with a flow rate of 0.5 mL/min and a column temperature of 50° C. Under the conditions used, 2-butanone and 2-butanol had retention times of 39.5 and 44.3 min, respectively. Alternatively, gas chromatography (GC) methods are available. For example, a specific GC method utilized an HP-INNOWax column (30 m×0.53 mm id, 1 μm film thickness, Agilent Technologies, Wilmington, Del.), with a flame ionization detector (FID). The carrier gas was helium at a flow rate of 4.5 mL/min, measured at 150° C. with constant head pressure; injector split was 1:25 at 200° C.; oven temperature was 45° C. for 1 min, 45 to 220° C. at 10° C./min, and 220° C. for 5 min; and FID detection was employed at 240° C. with 26 mL/min helium makeup gas. The retention times of 2-butanone and 2-butanol were 3.61 and 5.03 min, respectively.

2-Butanone can also be detected by derivatization with 3-methyl-2-benzothiazolinone hydrazone (MBTH). An aqueous solution containing 2-butanone is mixed with an equal volume of an aqueous solution of 6 mg/mL MBTH in 375 mM glycine-HCl (pH 2.7) and incubated at 100° C. for 3 min. The resulting MBTH-derivatized samples are analyzed on a 25 cm×4.6 mm (id) Supelosil LC-18-D5 5 μm column (Supelco) using a mobile phase of 55% acetonitrile in water at a flow rate of 1 mL/min. The 2-butanone derivative appears as two peaks (cis and trans isomers) with retention times of approximately 12.3 and 13.3 min and absorbance maxima of 230 and 307 nm.

The meaning of abbreviations is as follows: “s” means second(s), “min” means minute(s), “h” means hour(s), “psi” means pounds per square inch, “nm” means nanometers, “d” means day(s), “μL” means microliter(s), “mL” means milliliter(s), “L” means liter(s), “mm” means millimeter(s), “nm” means nanometers, “mM” means millimolar, “M” means molar, “mmol” means millimole(s), “μmol” means micromole(s)”, “g” means gram(s), “μg” means microgram(s) and “ng” means nanogram(s), “PCR” means polymerase chain reaction, “OD” means optical density, “OD₆₀₀” means the optical density measured at a wavelength of 600 nm, “kDa” means kilodaltons, “g” means the gravitation constant, “bp” means base pair(s), “kbp” means kilobase pair(s), “% w/v” means weight/volume percent, % v/v” means volume/volume percent, “wt %” means percent by weight, “nt” means not tested, “HPLC” means high performance liquid chromatography, and “GC” means gas chromatography. The term “molar selectivity” is the number of moles of product produced per mole of sugar substrate consumed and is reported as a percent.

### Example 1

### Increased Tolerance of Lactobacillus plantarum PN0512 to 1-Butanol, Iso-Butanol and 2-Butanol at Decreased Growth Temperatures

Tolerance levels of bacterial strain Lactobacillus plantarum PN0512 (ATCC # PTA-7727) were determined at 25° C., 30° C. and 37° C. as follows. The strain was cultured in S30L medium (i.e., 10 mM ammonium sulfate, 5 mM potassium phosphate buffer, pH 7.0, 50 mM MOPS, pH 7.0, 2 mM MgCl₂, 0.7 mM CaCl₂, 50 μM MnCl₂, 1 μM FeCl₃, 1 μM ZnCl₂, 1.72 μM CuCl₂, 2.53 μM COCl₂, 2.42 μM Na₂MoO₄, 2 μM thiamine hydrochloride, 10 mM glucose, and 0.2% yeast extract). An overnight culture in the absence of any test compound was started in 15 mL of the S30L medium in a 150 mL flask, with incubation at 37° C. in a shaking water bath. The next morning, the overnight culture was diluted into three 500 mL flasks containing 150 mL of fresh medium to an initial OD₆₀₀ of about 0.08. Each flask was incubated in a shaking water bath, one each at 25° C., 30° C. and 37° C. Each large culture was allowed to acclimate at the test temperature for at least 0.5 h. After the acclimation period, each large culture was split into flasks in the absence (control) and in the presence of various amounts of 1-butanol, isobutanol or 2-butanol, as listed in Tables 6, 7, and 8, respectively. Growth was followed by measuring OD₆₀₀ for six hours after addition of the compounds. The results are summarized in Tables 6, 7, and 8 below.

TABLE 6
Growth of L. plantarum PN0512 in the presence of 1-butanol at
different temperatures

| Concentration 1- | | | |
|---|---|---|---|
| butanol (% w/v) | 37° C. | 30° C. | 25° C. |
| 0.0 | +¹ | + | + |
| 1.0 | + | nt³ | nt |
| 1.2 | + | nt | nt |
| 1.4 | + | nt | nt |
| 1.5 | + | + | + |
| 1.6 | + | nt | nt |
| 1.8 | + | nt | nt |
| 2.0 | + | + | + |
| 2.1 | + | nt | nt |
| 2.2 | + | nt | nt |
| 2.3 | + | nt | nt |
| 2.4 | −² | + | + |
| 2.5 | − | nt | nt |
| 2.7 | − | + | nt |
| 2.9 | − | − | + |
| 3.1 | − | − | + |
| 3.2 | nt | − | − |
| 3.3 | nt | nt | − |
| 3.4 | nt | − | − |

¹“+” = growth observed as an increase in OD₆₀₀.
²“−” = no growth observed, i.e. no change in OD₆₀₀.
^{3“}nt” = not tested

TABLE 7
Growth of L. plantarum PN0512 in the presence of isobutanol at
different temperatures

| Concentration | | | |
|---|---|---|---|
| isobutanol (% | | | |
| w/v) | 37° C. | 30° C. | 25° C. |
| 0.0 | +¹ | + | + |
| 0.5 | + | nt³ | nt |
| 1.0 | + | nt | nt |
| 1.5 | + | + | + |
| 1.6 | + | nt | nt |
| 1.8 | + | nt | nt |
| 2.0 | + | + | + |
| 2.1 | + | nt | nt |
| 2.3 | + | nt | nt |
| 2.4 | + | + | + |
| 2.5 | + | nt | nt |
| 2.7 | + | + | + |
| 2.9 | + | + | + |
| 3.1 | + | + | + |
| 3.3 | nt | −² | + |
| 3.4 | − | nt | nt |
| 3.5 | nt | nt | + |
| 3.6 | nt | nt | − |
| 3.8 | − | nt | nt |
| 4.3 | − | nt | nt |

¹“+” = growth observed as an increase in OD₆₀₀.
²“−” = no growth observed, i.e. no change in OD₆₀₀.
^{3“}nt” = not tested

TABLE 8
Growth of L. plantarum PN0512 in the presence of 2-butanol at
different temperatures

| Concentration 2- | | | |
|---|---|---|---|
| butanol (% w/v) | 37° C. | 30° C. | 25° C. |
| 0.0 | +¹ | + | + |
| 1.8 | + | Nt³ | nt |
| 2.1 | + | nt | nt |
| 2.5 | + | nt | nt |
| 2.9 | + | + | + |
| 3.1 | + | nt | nt |
| 3.5 | + | nt | nt |
| 3.6 | + | nt | nt |
| 3.8 | + | + | + |
| 4.0 | nt | + | nt |
| 4.3 | + | + | + |
| 4.5 | −² | + | nt |
| 4.7 | − | + | + |
| 4.9 | nt | − | + |
| 5.2 | − | nt | + |
| 5.6 | − | nt | − |
| 6.0 | − | nt | nt |
| 6.4 | − | nt | nt |
| 7.3 | − | nt | nt |

¹“+” = growth observed as an increase in OD₆₀₀.
²“−” = no growth observed, i.e. no change in OD₆₀₀.
^{3“}nt” = not tested

All three butanols showed a similar effect of temperature on growth inhibition of L. plantarum PN0512. The concentration that resulted in full growth inhibition was greater at 25° C. than at 37° C. In the case of 1-butanol, growth was observed at 37° C. in 2.3% 1-butanol, but not 2.4%. However, at 30° C. growth was observed in 2.7%, but not 2.9%, and at 25° C. growth was observed even in 3.1% 1-butanol. Thus, the concentration of 1-butanol that completely inhibited growth increased as growth temperature decreased. Likewise, in the case of isobutanol, growth was observed in 3.5% at 25° C. while growth was observed in 3.1% at 30° C. and 37° C., but not in 3.3% or 3.4%. Similarly, in the case of 2-butanol growth was observed at 37° C. in 4.3%, but not in 4.5%; at 30° C. in 4.7%, but not in 4.9%; and at 25° C. in 5.2%. Thus the tolerance of L. plantarum PN0512 to butanols increased with decreased growth temperature.

### Example 2

### Increased Tolerance of Escherichia Coli to 1-Butanol at Decreased Exposure Temperature

The effect of growth and exposure temperature on survival of Escherichia coli in the presence of 1-butanol was tested using stationary phase cultures in a rich medium and log phase cultures in a defined medium. For the stationary phase studies, E. coli strain MG1655 (ATCC # 700926) was grown overnight in LB medium (Teknova, Half Moon Bay, Calif.) with shaking at 250 rpm at 42° C., 29° C. or 28° C. Survival of 1-butanol shock was tested at exposure temperatures of 0° C., 28° C. or 42° C. The 1-butanol exposure at 28° C. or 42° C. was started immediately after removing the overnight cultures from the growth incubators. The 1-butanol exposure at 0° C. was done after allowing the overnight cultures to cool on ice for about 15 min. A series of solutions of 1-butanol at different concentrations in LB medium was made and 90 μL aliquots were put in microfuge tubes. To these were added 10 μL of the overnight cultures and the tubes were immediately placed in shaking incubators at 42° C. or 28° C. or left on ice for 30 min. To stop the effect of 1-butanol on the cultures, a 10⁻² dilution was done by placing 2 μL of the treated culture into 198 μL of LB medium in wells of a microplate. Then, 5 μL of the undiluted treated cultures were spotted on LB agar plates. Subsequent 10-fold serial dilutions of 10⁻³, 10⁻⁴, 10⁻⁵ and 10⁻⁶ of the exposed cultures were done by serial pipetting of 20 μL, starting with the 10⁻² dilution cultures, into 180 μL of LB medium in the microplate, using a multi-channel pipette. Prior to each transfer, the cultures were mixed by pipetting up and down six times. Each dilution (5 μL) was spotted onto an LB plate using a multi-channel pipette and allowed to soak into the plate. The plates were inverted and incubated overnight at 37° C. The number of colonies for each dilution was counted and the % growth inhibition was calculated by comparison with a control culture that had not been exposed to 1-butanol. Survival of 0% was recorded when no colonies in the spots of the undiluted or any of the serial dilutions were observed. The results are shown in Table 9.

TABLE 9
Survival of stationary phase E. coli in 1-butanol
at 42° C., 28° C., or 0° C.

| | Grown | Grown | Grown | Grown | Grown | Grown |
|---|---|---|---|---|---|---|
| | at | at | at | at | at | at |
| | 42° C. | 29° C. | 42° C. | 28° C. | 42° C. | 29° C. |
| | % survival | | % survival | | % survival | |
| | after 30 min | | after 30 min | | after 30 min | |
| 1-Butanol | exposure at | | exposure at | | exposure at | |
| % (w/v) | 42° C. | | 28° C. | | 0° C. | |
| 1.0 | 100 | 100 | 100 | 100 | 100 | 100 |
| 1.5 | 0.1 | 0.1 | 100 | 100 | 100 | 100 |
| 2.0 | 0 | 0.1 | 100 | 100 | 100 | 100 |
| 2.5 | 0 | 0 | 100 | 100 | 100 | 100 |
| 3.0 | 0 | 0 | 100 | 100 | 100 | 100 |
| 3.5 | 0 | 0 | 3 | 10 | 100 | 100 |
| 4.0 | 0 | 0 | 0.0004 | 0.0003 | 100 | 100 |
| 5.0 | nt¹ | nt | nt | nt | 1 | 1 |
| 6.0 | nt | nt | nt | nt | 0 | 0.001 |
| 7.0 | nt | nt | nt | nt | 0 | 0 |

¹“nt” = not tested

A similar study was done with log-phase cultures of E. coli grown in a defined medium. E. coli strain MG1655 was allowed to grow overnight in MOPS 0.2% glucose medium (Teknova, Half Moon Bay, Calif.) at 42° C. or 28° C. The following day, the cultures were diluted into fresh medium and allowed to grow at the same temperature until in the log phase of growth. The OD₆₀₀ was 0.74 for the 28° C. culture and was 0.72 for the 42° C. culture. Both of these log phase cultures were exposed to 1-butanol at 42° C., 28° C. and 0° C. as follows. A series of solutions of 1-butanol at different concentrations in MOPS 0.2% glucose medium was made and 90 μL aliquots were put in microfuge tubes. To these were added 10 μL of the log phase cultures and the tubes were immediately placed in shaking incubators at 42° C. or 28° C. or left on ice for 30 min. To stop the effect of 1-butanol on the cultures, a 10⁻² dilution was done by placing 2 μL of the treated culture into 198 μL of LB medium in wells of a microplate. Then 5 μL of the undiluted treated cultures were spotted on LB agar plates. Subsequent 10-fold serial dilutions of 10⁻³, 10⁻⁴, 10⁻⁵ and 10⁻⁶ of the exposed cultures were done by serial pipetting of 20 μL, starting with the 10⁻² dilution cultures, into 180 μL of LB medium in the microplate, using a multi-channel pipette. Prior to each transfer, the cultures were mixed by pipetting up and down six times. Each dilution (5 μL) was spotted onto an LB plate using a multi-channel pipette and allowed to soak into the plate. The plates were inverted and incubated overnight at 37° C. The number of colonies for each dilution was counted and the % growth inhibition was calculated by comparison with a control culture that had not been exposed to 1-butanol. Survival of 0% was recorded when no colonies in the spots of the undiluted or any of the serial dilutions were observed. The results are shown in Table 10.

TABLE 10
Survival of log-phase E. coli in 1-butanol at
42° C., 28° C., or 0° C.

| | Grown | Grown | | Grown | Grown | |
|---|---|---|---|---|---|---|
| | at | at | Grown at | at | at | Grown at |
| | 42° C. | 28° C. | 42° C. | 28° C. | 42° C. | 29° C. |
| | % survival | | % survival | | % survival | |
| | after 30 min | | after 30 min | | after 30 min | |
| 1-Butanol | exposure at | | exposure at | | exposure at | |
| % (w/v) | 42° C. | | 28° C. | | 0° C. | |
| 1.0 | 100 | 100 | nt | nt | nt | nt |
| 1.5 | 0 | 0 | 100 | 100 | nt | nt |
| 2.0 | 0 | 0 | 100 | 100 | nt | nt |
| 2.5 | 0 | 0 | 0.1 | 50 | 100 | 100 |
| 3.0 | 0 | 0 | 0 | 0 | 100 | 100 |
| 3.5 | 0 | 0 | 0.01 | 0 | 100 | 100 |
| 4.0 | 0 | 0 | 0.001 | 0 | 100 | 100 |
| 4.5 | nt¹ | nt | 0 | 0 | 100 | 100 |
| 5.0 | nt | nt | nt | nt | 10 | 50 |
| 6.0 | nt | nt | nt | nt | 1 | 1 |

¹“nt” = not tested

For both the stationary phase and log-phase cultures of E. coli MG1655, the growth temperature had very little, if any, effect on the survival of a 1-butanol shock. However, the exposure temperature had a major effect on the survival of E. coli to 1-butanol shock. As can be seen from the data in Tables 9 and 10, the tolerance of E. coli MG1655 to 1-butanol increased with decreasing exposure temperature.

### Example 3

### Increased Tolerance of Escherichia coli to 2-Butanone at Decreased Exposure Temperature

The effect of exposure temperature on survival of Escherichia coli in the presence of 2-butanone (also referred to herein as methyl ethyl ketone or MEK) was tested as follows. E. coli strain BW25113 (The Coli Genetic Stock Center (CGSC), Yale University; # 7636) was grown overnight in LB medium (Teknova, Half Moon Bay, Calif.) with shaking at 250 rpm at 37° C. Survival of MEK shock was tested at exposure temperatures of 28° C. or 37° C. A series of solutions of MEK at different concentrations in LB medium was made and 90 μL aliquots were put in microfuge tubes. To these were added 10 μL of the overnight culture and the tubes were immediately placed in shaking incubators at 37° C. or 28° C. for 30 min. To stop the effect of MEK on the cultures, a 10⁻² dilution was done by placing 2 μL of the MEK treated culture into 198 μL of LB medium in wells of a microplate. Then 5 μL of the undiluted treated cultures were spotted on LB agar plates. Subsequent 10-fold serial dilutions of 10⁻³, 10⁻⁴, 10⁻⁵ and 10⁻⁶ of the exposed cultures were done by serial pipetting of 20 μL, starting with the 10⁻² dilution cultures, into 180 μL of LB medium in the microplate, using a multi-channel pipette. Prior to each transfer, the cultures were mixed by pipetting up and down six times. Each dilution (5 μL) was spotted onto LB plates using a multi-channel pipette and allowed to soak into the plate. The plates were inverted and incubated overnight at 37° C. The number of colonies for each dilution was counted and the % growth inhibition was calculated by comparison with a control culture that had not been exposed to MEK. Survival of 0% was recorded when no colonies in the spots of the undiluted or any of the serial dilutions were observed. The results, given as the average of duplicate experiments, are shown in Table 11.

TABLE 11
Survival of E. coli in MEK at 37° C. and 28° C.

| MEK % w/v | % Survival at 37° C. | % Survival at 28° C. |
|---|---|---|
| 0 | 100 | 100 |
| 4 | 100 | 100 |
| 6 | 0 | 100 |
| 8 | 0 | 0.002 |

Reducing the exposure temperature from 37° C. to 28° C. dramatically improved survival of E. coli to MEK treatment. At 37° C. there was full survival at 4% w/v and no survival at 6% w/v, while at 28° C. there was full survival at 6% w/v. Thus, the tolerance of E. coli to MEK increased with decreasing exposure temperature.

### Example 4

### Increased Tolerance of E. coli and L. Plantarum PN0512 to 1-Butanol at Decreased Exposure Temperature

This Example demonstrates that the toxic effects of 1-butanol and 2-butanol on various microbial cells was reduced at lower temperatures. This was demonstrated by incubating E. coli (strain MG1655; ATCC # 700926), and L. plantarum (strain PN0512; ATCC # PTA-7727) with either 1-butanol or 2-butanol at different temperatures and then determining the fraction of the cells that survived the treatment at the different temperatures.

Using overnight cultures or cells from plates, 30 mL cultures of the microorganisms to be tested were started in the following culture media:
E. coli—Miller's LB medium (Teknova, Half Moon Bay, Calif.):
L. plantarum PN0512—Lactobacilli MRS Broth (BD Diagnostic Systems, Sparks, Md.).
   The E. coli and L. plantarum cultures were grown at 37° C. aerobically with shaking until the cultures were in log phase and the OD₆₀₀ was between 0.6 and 0.8. A 50 μL aliquot of each culture was removed for a time zero sample. The remainder of the cultures was divided into six 5 mL portions and placed in six small incubation flasks or tubes. Different amounts of 1-butanol or 2-butanol were added to the six flasks to bring the concentration to predetermined values, as listed in the tables below. The flasks or tubes were incubated at a desired temperature, aerobically without shaking for 1 h. After the incubation with one of the butanols, 2 μL from each of the flasks (and in addition 2 μL of the time zero sample of the culture before exposure to one of the butanols) were pipetted into the “head” wells of a 96 well (8×12) microtiter plate, each containing 198 μL of LB medium to give a 10⁻² dilution of the culture. Subsequently, 10⁻³, 10⁻⁴, 10⁻⁵, and 10⁻⁶ serial dilutions of the cultures were prepared as follows. The 10⁻³ dilution was prepared by pipetting 20 μL of the sample from the head well into the 180 μL LB medium in the next well using a multi-channel pipette. This procedure was repeated 3 more times on successive wells to prepare the 10⁻⁴, 10⁻⁵, and 10⁻⁶ dilutions. After each liquid transfer, the solution in the well was mixed by pipetting it up and down 10 times with the multi-channel pipetor. A 5 μL aliquot of each dilution was spotted onto an LB plate using a multi-channel pipette starting with the 10⁻⁶ dilution, then the 10⁻⁵, and so on working from more to less dilute without a change of tips. The spots were allowed to soak into the agar by leaving the lid of the plate slightly open for 15 to 30 min in a sterile transfer hood. The plates were covered, inverted, and incubated overnight at 37° C. The following day, the number of colonies in the spots were counted from the different dilutions. The number of living cells/mL in each of the original culture solutions from which the 2 μL was withdrawn was calculated and compared to the number of cells in the control untreated culture to determine the % of the cells surviving.

The results of experiments in which E. coli cells were treated with 1-butanol at temperatures of 0, 30, and 37° C. are shown Table 12.

TABLE 12
Percentage of E. coli cells surviving in 1-butanol at 0, 30 and 37° C.

| 1-butanol | % Survival | % Survival | % Survival |
|---|---|---|---|
| % v/v | at 0° C. | at 30° C. | at 37° C. |
| 0 | 100 | 100 | 100 |
| 1 | nt¹ | 100 | 72 |
| 1.5 | nt | 100 | 20 |
| 2 | nt | 100 | 0 |
| 2.5 | 100 | 23 | 0 |
| 3 | 100 | 0 | 0 |
| 3.5 | 100 | 0 | nt |
| 4 | 100 | nt | nt |
| 4.5 | 100 | nt | nt |

^{1“}nt” = not tested

The concentration at which 1-butanol kills E. coli cells was affected by the treatment temperature. At 0° C., concentrations of 1-butanol as high as 4.5% v/v had no toxic effect on E. coli cells during a one hour treatment. At 30° C., E. coli cells were killed when treated with 3% v/v 1-butanol for one hour. At 37° C., E. coli cells were killed when treated with 2% v/v 1-butanol for one hour.

The results of experiments in which L. plantarum PN0512 cells were treated with 1-butanol at temperatures of 0, 23, and 37° C. for one hour are shown Table 13.

TABLE 13
Percentage of L. plantarum PN0512 cells surviving in 1-butanol
at 0, 23 and 37° C.

| 1-butanol | % Survival | % Survival | % Survival |
|---|---|---|---|
| % v/v | at 0° C. | at 23° C. | at 37° C. |
| 0 | 100 | 100 | 100 |
| 1 | nt¹ | nt | 80 |
| 1.5 | nt | nt | 58 |
| 2 | nt | 100 | 29 |
| 2.5 | nt | 100 | 8 |
| 3 | 100 | 82 | 0 |
| 3.5 | 100 | 0 | 0 |
| 4 | 100 | 0 | nt |
| 4.5 | 100 | 0 | nt |
| 5 | 0 | nt | nt |
| 5.5 | 0 | nt | nt |

^{1“}nt” = not tested

The concentration at which 1-butanol kills L. plantarum PN0512 cells was affected by the treatment temperature. At 0° C., concentrations of 1-butanol as high as 4.5% v/v had no toxic effect on L. plantarum PN0512 cells during a one hour treatment. At 23° C., L. plantarum PN0512 cells were killed when treated with 3.5% v/v 1-butanol for one hour. At 37° C., L. plantarum PN0512 cells were killed when treated with 2.5% v/v 1-butanol for one hour.

### Example 5

### Cloning and Expression of Acetolactate Synthase

The purpose of this Example was to clone and express in E. coli the budB gene that encodes the enzyme acetolactate synthase. The budB gene was amplified from Klebsiella pneumoniae strain ATCC 25955 genomic DNA using PCR.

The budB sequence which encodes acetolactate synthase was amplified from Klebsiella pneumoniae (ATCC 25955) genomic DNA by PCR using the primer pair B1 (SEQ ID NO:15) and B2 (SEQ ID NO:16). Other PCR amplification reagents (e.g. Kod HiFi DNA Polymerase (Novagen Inc., Madison, Wis.; catalog no. 71805-3)) were supplied in manufacturers' kits and used according to the manufacturer's protocol. Klebsiella pneumoniae genomic DNA was prepared using the Gentra Puregene Puregene kit (Gentra Systems, Inc., Minneapolis, Minn.; catalog number D-5000A). Amplification was carried out in a DNA Thermocycler GeneAmp 9700 (PE Applied Biosystems, Foster city, CA). The nucleotide sequence of the open reading frame (ORF) and the predicted amino acid sequence of the enzyme are given as SEQ ID NO:3 and SEQ ID NO:4, respectively.

For expression studies the Gateway cloning technology (Invitrogen Corp., Carlsbad, Calif.) was used. The entry vector pENTR/SD/D-TOPO allows directional cloning and provided a Shine-Dalgarno sequence for the gene of interest. The destination vector pDEST14 used a T7 promoter for expression of the gene with no tag. The forward primer incorporated four bases (CACC) immediately adjacent to the translational start codon to allow directional cloning of the budB acetolactate synthase coding region PCR product into pENTR/SD/D-TOPO (Invitrogen), generating the plasmid pENTRSDD-TOPObudB. The PENTR construct was transformed into E. coli Top10 (Invitrogen) cells and plated according to the manufacturer's recommendations. Transformants were grown overnight and plasmid DNA was prepared using the QIAprep Spin Miniprep kit (Qiagen, Valencia, Calif.; catalog no. 27106) according to the manufacturer's recommendations. To create an expression clone, the budB coding region from PENTRSDD-TOPObudB was transferred to the PDEST 14 vector by in vitro recombination using the LR Clonase mix (Invitrogen, Corp., Carlsbad, Calif.). The resulting vector, pDEST14budB, was transformed into BL-21-Al cells (Invitrogen Corp.). BL-21-Al cells carry a chromosomal copy of the T7 RNA polymerase under control of the arabinose-inducible araBAD promoter.

Transformants are inoculated into LB medium supplemented with 50 μg/mL of ampicillin and grown overnight. An aliquot of the overnight culture is used to inoculate 50 mL of LB medium supplemented with 50 μg/mL of ampicillin. The culture is incubated at 37° C. with shaking until the OD₆₀₀ reaches 0.6-0.8. The culture is split into two 25-mL portions and arabinose is added to one of the flasks to a final concentration of 0.2% w/v. The negative control flask is not induced with arabinose. The flasks are incubated for 4 h at 37° C. with shaking. Cells are harvested by centrifugation and the cell pellets are resuspended in 50 mM MOPS, pH 7.0 buffer. The cells are disrupted either by sonication or by passage through a French Pressure Cell. Each cell lysate is centrifuged yielding the supernatant and the pellet or the insoluble fraction. An aliquot of each fraction (whole cell lysate, from induced and control cells, is resuspended in SDS (MES) loading buffer (Invitrogen), heated to 85° C. for 10 min and subjected to SDS-PAGE analysis (NuPAGE 4-12% Bis-Tris Gel, catalog no. NP0322Box, Invitrogen). A protein of the expected molecular weight, as deduced from the nucleic acid sequence, is present in the induced culture but not in the uninduced control.

Acetolactate synthase activity in the cell free extracts is measured using the method described by Bauerle et al. (Bauerle et al. (1964) Biochim. Biophys. Acta 92:142-149). Protein concentration is measured by either the Bradford method or by the Bicinchoninic Kit (Sigma, catalog no. BCA-1; St. Louis, Mo.) using Bovine serum albumin (BSA) (Bio-Rad, Hercules, Calif.) as the standard.

### Example 6

### Cloning and Expression of Acetolactate Decarboxylase

The purpose of this Example was to clone and express in E. coli the budA gene that encodes the enzyme acetolactate decarboxylase. The budA gene was amplified from Klebsiella pneumoniae strain ATCC 25955 genomic DNA using PCR.

The budA sequence which encodes acetolactate decarboxylase, was cloned in the same manner as described for budB in Example 5, except that the primers used for PCR amplification were B3 (SEQ ID NO:17) and B4 (SEQ ID NO:18). The nucleotide sequence of the open reading frame (ORF) and the predicted amino acid sequence of the enzyme are given as SEQ ID NO:1 and SEQ ID NO:2, respectively. The resulting plasmid was named pENTRSDD-TOPObudA.

Acetolactate decarboxylase activity in the cell free extracts is measured using the method described by Bauerle et al., supra.

### Example 7

### Prophetic

### Cloning and Expression of Butanediol Dehydrogenase

The purpose of this prophetic Example is to describe how to clone and express in E. coli the budC gene that encodes the enzyme butanediol dehydrogenase. The budC gene is amplified from Klebsiella pneumoniae strain IAM1063 genomic DNA using PCR.

The budC sequence encoding butanediol dehydrogenase is cloned and expressed in the same manner as described for budA in Example 5, except that the primers used for PCR amplification are B5 (SEQ ID NO:19) and B6 (SEQ ID NO:20) and the genomic template DNA is from Klebsiella pneumoniae IAM1063 (which is obtained from the Institute of Applied Microbiology Culture Collection, Tokyo, Japan). Klebsiella pneumoniae IAM1063 genomic DNA is prepared using the Gentra Puregene Puregene kit (Gentra Systems, Inc., Minneapolis, Minn.; catalog number D-5000A). The nucleotide sequence of the open reading frame (ORF) and the predicted amino acid sequence of the enzyme are given as SEQ ID NO:5 and SEQ ID NO:6, respectively.

Butanediol dehydrogenase activity in the cell free extracts is measured spectrophotometrically by following NADH consumption at an absorbance of 340 nm.

### Example 8

### Prophetic

### Cloning and Expression of Butanediol Dehydratase

The purpose of this prophetic Example is to describe how to clone and express in E. coli the pddA, pddB and pddC genes that encode butanediol dehydratase. The pddA, pddB and pddC genes are amplified from Klebsiella oxytoca ATCC 8724 genomic DNA using PCR.

The pddA, pddB and pddC sequences which encode butanediol dehydratase are cloned and expressed in the same manner as described for budA in Example 5, except that the genomic template DNA is from Klebsiella oxytoca ATCC 8724, and the primers are B7 (SEQ ID NO:21) and B8 (SEQ ID NO:22). Klebsiella oxytoca genomic DNA is prepared using the Gentra Puregene Puregene kit (Gentra Systems, Inc., Minneapolis, Minn.; catalog number D-5000A). A single PCR product including all three open reading frames (ORFs) is cloned, so that all three coding regions are expressed as an operon from a single promoter on the expression plasmid. The nucleotide sequences of the open reading frames for the three subunits are given as SEQ ID NOs:7, 9, and 11, respectively, and the predicted amino acid sequences of the three enzyme subunits are given as SEQ ID NOs:8, 10, and 12, respectively.

Butanediol dehydratase activity in the cell free extracts is measured by derivatizing the ketone product with 2,4-dinitrophenylhydrazine (DNPH). Briefly, 100 μL of reaction mixture, cell extract containing approximately 0.0005 units of enzyme, 40 mM potassium phosphate buffer (pH 8.0), 2 μg of adenosylcobalamin, 5 μg of 2,3,-butanediol, and 1 μg of bovine serum albumin, is quenched by addition of an equal volume of 0.05 wt % DNPH in 1.0 N HCl. After 15 min at room temperature, the color is developed by addition of 100 μL of 4 N NaOH. The amount of product is determined from the absorbance of the final solution at 550 nm compared to a standard curve prepared with 2-butanone. All reactions are carried out at 37° C. under dim red light.

### Example 9

### Prophetic

### Cloning and Expression of Butanol Dehydrogenase

The purpose of this prophetic Example is to describe how to clone and express in E. coli the sadh gene that encodes butanol dehydrogenase. The sadh gene is amplified from Rhodococcus ruber strain 219 genomic DNA using PCR.

The sadh sequence encoding butanol dehydrogenase is cloned and expressed in the same manner as described for budA in Example 5, except that the genomic template DNA is from Rhodococcus ruber strain 219 (Meens, Institut fuer Mikrobiologie, Universitaet Hannover, Hannover, Germany) and the primers are B9 (SEQ ID NO:23) and B10 (SEQ ID NO:24). Rhodococcus rubergenomic DNA is prepared using the Ultra Clean™ Microbial DNA Isolation Kit (MO BIO Laboratories Inc., Carlsbad, Calif.), according to the manufacturer's protocol. The nucleotide sequence of the open reading frame (ORF) and the predicted amino acid sequence of the enzyme are given as SEQ ID NO:13 and SEQ ID NO:14, respectively.

Butanol dehydrogenase activity in cell free extracts is measured by following the increase in absorbance at 340 nm resulting from the conversion of NAD to NADH when the enzyme is incubated with NAD and 2-butanol.

### Example 10

### Prophetic

### Construction of a Transformation Vector for the Genes in a 2-Butanol Biosynthetic Pathway

The purpose of this prophetic Example is to describe the preparation of a transformation vector for the genes in a 2-butanol biosynthetic pathway (i.e., Pathway 3 as described above). Like most organisms, E. coli converts glucose initially to pyruvic acid. The enzymes required to convert pyruvic acid to 2-butanol following Pathway 3, i.e., acetolactate synthase, acetolactate decarboxylase, butanediol dehydrogenase, butanediol dehydratase, and butanol dehydrogenase, are encoded by the budA, budB, budC, pddA, pddB, pddC and sadh genes. To simplify building the 2-butanol biosynthetic pathway in a recombinant organism, the genes encoding the 5 steps in the pathway are divided into two operons. The upper pathway comprises the first three steps catalyzed by acetolactate synthase, acetolactate decarboxylase, and butanediol dehydrogenase. The lower pathway comprises the last two steps catalyzed by butanediol dehydratase and butanol dehydrogenase.

The coding sequences are amplified by PCR with primers that incorporate restriction sites for later cloning, and the forward primers contain an optimized E. coli ribosome binding site (AAAGGAGG). PCR products are TOPO cloned into the pCR4 Blunt-TOPO vector and transformed into Top10 cells (Invitrogen). Plasmid DNA is prepared from the TOPO clones, and the sequence of the cloned PCR fragment is verified. Restriction enzymes and T4 DNA ligase (New England Biolabs, Beverly, Mass.) are used according to manufacturer's recommendations. For cloning experiments, restriction fragments are gel-purified using QIAquick Gel Extraction kit (Qiagen).

After confirmation of the sequence, the coding regions are subcloned into a modified pUC19 vector as a cloning platform. The pUC19 vector is modified by a HindIII/Sapi digest, followed by treatment with Klenow DNA polymerase to fill in the ends. The 2.4 kB vector fragment is gel-purified and religated creating pUC19dHS. Alternatively the pUC19 vector is modified by a SphI/SapI digest, followed by treatment with Klenow DNA polymerase to blunt the ends. The 2.4 kB vector fragment is gel-purified and religated creating pUC19dSS. The digests remove the lac promoter adjacent to the MCS (multiple cloning sites), preventing transcription of the operons from the vector.

### Upper Pathway:

The budABC coding regions are amplified from Klebsiella pneumoniae genomic DNA by PCR using primer pair B11 and B12 (Table 4), given as SEQ ID NOs:25 and 26, respectively. The forward primer incorporates an EcoRI restriction site and a ribosome binding site (RBS). The reverse primer incorporates an SphI restriction site. The PCR product is cloned into pCR4 Blunt-TOPO creating pCR4 Blunt-TOPO-budABC.

To construct the upper pathway operon pCR4 Blunt-TOPO-budABC is digested with EcoRI and SphI releasing a 3.2 kbp budABC fragment. The pUC19dSS vector is also digested with EcoRI and SphI, releasing a 2.0 kbp vector fragment. The budABC fragment and the vector fragment are ligated together using T4 DNA ligase (New England Biolabs) to form pUC19dSS-budABC.

### Lower Pathway:

The pddABC coding regions are amplified from Klebsiella oxytoca ATCC 8724 genomic DNA by PCR using primers B13 and B14 (Table 4), given as SEQ ID NOs:27 and 28, respectively, creating a 2.9 kbp product. The forward primer incorporates EcoRI and PmeI restriction sites and a RBS. The reverse primer incorporates the BamHI restriction site. The PCR product is cloned into pCRBlunt II-TOPO creating pCRBluntII-pdd.

The sadh gene is amplified from Rhodococcus ruber strain 219 genomic DNA by PCR using primers B15 and B16 (Table 4), given as SEQ ID NOs:29 and 30, respectively, creating a 1.0 kbp product. The forward primer incorporates a BamHI restriction site and a RBS. The reverse primer incorporates an XbaI restriction site. The PCR product is cloned into pCRBlunt II-TOPO creating pCRBluntII-sadh.

To construct the lower pathway operon, a 2.9 kbp EcoRI and BamHI fragment from pCRBluntII-pdd, a 1.0 kbp BamHI and XbaI fragment from pCRBluntII-sadh, and the large fragment from an EcoRI and XbaI digest of pUC19dHS are ligated together. The three-way ligation creates pUC19dHS-pdd-sadh.

The pUC19dSS-budABC vector is digested with PmeI and HindIII, releasing a 3.2 kbp fragment that is cloned into pBenBP, an E. coli-B. subtilis shuttle vector. Plasmid pBenBP is created by modification of the pBE93 vector, which is described by Nagarajan (WO 93/2463, Example 4). To generate pBenBP, the Bacillus amyloliquefaciens neutral protease promoter (NPR) signal sequence and the phoA gene are removed from pBE93 with an NcoI/HindIII digest. The NPR promoter is PCR amplified from pBE93 by primers BenF and BenBPR, given by SEQ ID NOs:31 and 32, respectively. Primer BenBPR incorporates BstEII, PmeI and HindIII sites downstream of the promoter. The PCR product is digested with NcoI and HindIII, and the fragment is cloned into the corresponding sites in the vector pBE93 to create pBenBP. The upper operon fragment is subcloned into the PmeI and HindIII sites in pBenBP creating pBen-budABC.

The pUC19dHS-pdd-sadh vector is digested with PmeI and HindIII releasing a 3.9 kbp fragment that is cloned into the PmeI and HindIII sites of pBenBP, creating pBen-pdd-sadh.

### Example 11

### Prophetic

### Expression of a 2-Butanol Biosynthetic Pathway in E. coli

The purpose of this prophetic Example is to describe how to express a 2-butanol biosynthetic pathway in E. coli.

The plasmids pBen-budABC and pBen-pdd-sadh, prepared as described in Example 10, are separately transformed into E. coli NM522 (ATCC No. 47000), and expression of the genes in each operon is monitored by SDS-PAGE analysis and enzyme assay. After confirmation of expression of all genes, pBen-budABC is digested with EcoRI and HindIII to release the NPR promoter-budABC fragment. The fragment is blunt ended using the Klenow fragment of DNA polymerase (New England Biolabs, catalog no. M0210S). The plasmid pBen-pdd-sadh is digested with EcoRI and similarly blunted to create a linearized, blunt-ended vector fragment. The vector and NPR-budABC fragments are ligated, creating p2BOH. This plasmid is transformed into E. coli NM522 to give E. coli NM522/p2BOH, and expression of the genes is monitored as previously described.

E. coli NM522/p2BOH is inoculated into a 250 mL shake flask containing 50 mL of medium and shaken at 250 rpm and 35° C. The medium is composed of: dextrose, 5 g/L; MOPS, 0.05 M; ammonium sulfate, 0.01 M; potassium phosphate, monobasic, 0.005 M; S10 metal mix, 1% (v/v); yeast extract, 0.1% (w/v); casamino acids, 0.1% (w/v); thiamine, 0.1 mg/L; proline, 0.05 mg/L; and biotin 0.002 mg/L, and is titrated to pH 7.0 with KOH. S10 metal mix contains: MgCl₂, 200 mM; CaCl₂, 70 mM; MnCl₂, 5 mM; FeCl₃, 0.1 mM; ZnCl₂, 0.1 mM; thiamine hydrochloride, 0.2 mM; CuSO₄, 172 μM; COCl₂, 253 μM; and Na₂MoO₄, 242 μM. After 18 h, 2-butanol is detected by HPLC or GC analysis using methods that are well known in the art, for example, as described in the General Methods section above.

### Example 12

### Prophetic

### Expression of a 2-Butanol Biosynthetic Pathway in Bacillus subtilis

The purpose of this prophetic Example is to describe how to express a 2-butanol biosynthetic pathway in Bacillus subtilis.

The plasmids pBen-budABC and pBen-pdd-sadh, prepared as described in Example 10, are separately transformed into Bacillus subtilis BE1010 (J. Bacteriol. 173:2278-2282 (1991)) and expression of the genes in each operon is monitored as described in Example 11. The plasmid pBen-budABC is digested with EcoRI and HindIII to release the NPR promoter-budABC fragment. The fragment is blunt ended using the Klenow fragment of DNA polymerase (New England Biolabs, catalog no. M0210S). The plasmid pBen-pdd-sadh is digested with EcoRI and similarly blunted to create a linearized, blunt-ended vector fragment. The vector and NPR-budABC fragments are ligated, creating p2BOH. This plasmid is transformed into Bacillus subtilis BE1010 to give Bacillus subtilis BE1010/p2BOH, and expression of the genes is monitored as previously described.

Bacillus subtilis BE1010/p2BOH is inoculated into a 250 mL shake flask containing 50 mL of medium and shaken at 250 rpm and 35° C. for 18 h. The medium is composed of: dextrose, 5 g/L; MOPS, 0.05 M; glutamic acid, 0.02 M; ammonium sulfate, 0.01 M; potassium phosphate, monobasic buffer, 0.005 M; S10 metal mix (as described in Example 11), 1% (v/v); yeast extract, 0.1% (w/v); casamino acids, 0.1% (w/v); tryptophan, 50 mg/L; methionine, 50 mg/L; and lysine, 50 mg/L, and is titrated to pH 7.0 with KOH. After 18 h, 2-butanol is detected by HPLC or GC analysis using methods that are well known in the art, for example, as described in the General Methods section above.

### Example 13

### Construction of a Transformation Vector for the Genes in a 2-Butanol Biosynthetic Pathway

The purpose of this Example was to prepare a recombinant E. coli host carrying the genes in a 2-butanol biosynthetic pathway (i.e., Pathway 3 as described above). Like most organisms, E. coli converts glucose initially to pyruvic acid. The enzymes required to convert pyruvic acid to 2-butanone in Pathway 3, i.e., acetolactate synthase, acetolactate decarboxylase, butanediol dehydrogenase, and butanediol dehydratase are encoded by the budA, budB, budC, pddA, pddB, and pddC genes. In the last step of the pathway, a butanol dehydrogenase converts 2-butanone to 2-butanol. Dehydrogenases that carry out this last step are promiscuous and may be found in many organisms. To simplify building the 2-butanol biosynthetic pathway in a recombinant organism, the genes encoding the 5 steps in the pathway were divided into multiple operons. The upper pathway operon comprised the first three steps catalyzed by acetolactate synthase, acetolactate decarboxylase, and butanediol dehydrogenase and were cloned onto an expression vector. The lower pathway comprised the last two steps catalyzed by butanediol dehydratase including the reactivating factor (Mori et al., J. Biol. Chem. 272:32034 (1997)) and a butanol dehydrogenase. The diol dehydratase can undergo suicide inactivation during catalysis. The reactivating factor protein encoded by ddrA and ddrB (GenBank AF017781, SEQ ID NO:70) reactivates the inactive enzyme. The ddrA and ddrB genes flank the diol dehydratase operon. The operons for the dehydratase/reactivating factor and the butanol dehydrogenase were either cloned onto another expression vector or the dehydratase/reactivating factor operon was cloned singly onto another expression vector and the last step was provided by an endogenous activity in the demonstration host.

Construction of Vector pTrc99a-budABC:

The budAB coding regions were amplified from K. pneumoniae ATCC 25955 genomic DNA by PCR using primer pair BABC F and BAB R, given as SEQ ID NOs:33 and 34, respectively (see Table 4), creating a 2.5 kbp product. The forward primer incorporated SacI and EcoRI restriction sites and a ribosome binding site (RBS). The reverse primer incorporated a SpeI restriction site. The PCR product was cloned into pCR4 Blunt-TOPO creating pCR4 Blunt-TOPO-budAB. Plasmid DNA was prepared from the TOPO clones and the sequence of the genes was verified with primers M13 Forward (SEQ ID NO:35), M13 Reverse (SEQ ID NO:36), N83 SeqF2 (SEQ ID NO:37), N83 SeqF3 (SEQ ID NO:38) and N84 SeqR4 (SEQ ID NO:39) (see Table 5).

The budC coding region was amplified from K. pneumoniae ATCC 25955 genomic DNA by PCR using primer pair BC Spe F and BC Xba R given as SEQ ID NOs:40 and 41, respectively, creating a 0.8 kbp product. The forward primer incorporated a SpeI restriction site, a RBS and modified the CDS by changing the second and third codons from AAA to AAG. The reverse primer incorporated an XbaI restriction site. The PCR product was cloned into pCR4 Blunt-TOPO creating pCR4 Blunt-TOPO-budC. Plasmid DNA was prepared from the TOPO clones and the sequence of the genes was verified with primers M13 Forward (SEQ ID NO:35) and M13 Reverse (SEQ ID NO:36).

To construct the budABC operon, pCR4 Blunt-TOPO-budC was digested with SnaBI and XbaI releasing a 1.0 kbp budC fragment. The vector pTrc99a (Amann et al., Gene 69(2):301-315 (1988)) was digested with SmaI and XbaI creating a 4.2 kbp linearized vector fragment. The vector and the budC fragment were ligated to create pTrc99a-budC and transformed into E. coli Top 10 cells (Invitrogen). Transformants were analyzed by PCR amplification with primers Trc F (SEQ ID NO:42) and Trc R (SEQ ID NO:43) for a 1.2 kbp product to confirm the presence of the budC insert. The budAB genes were subcloned from pCR4 Blunt-TOPO-budAB as a 2.5 kbp EcoRI/SpeI fragment. Vector pTrc99a-budC was digested with EcoRI and SpeI and the resulting 5.0 kbp vector fragment was gel-purified. The purified vector and budAB insert were ligated and transformed into E. coli Top 10 cells. Transformants were screened by PCR amplification with primers Trc F (SEQ ID NO:42) and N84 Seq R2 (SEQ ID NO:65) to confirm creation of pTrc99a-budABC. In this plasmid, the bud A, B, and C coding regions are adjacent to each other, in this order, and between the Trc promoter and the rrnB termination sequence.

### Results:

Three independent isolates of E. coli Top 10/pTrc99a-budABC were examined for the production of butanediol, using E. coli Top 10/pCL1925-Kodd-ddr (described below) as a negative control. The strains were grown in LB medium containing 100 μg/mL carbenicillin. The resulting cells were used to inoculate shake flasks (approximately 175 mL total volume) containing 125 mL of TM3a/glucose medium with 100 μg/mL carbenicillin. In addition, the flasks inoculated with strains carrying pTrc99a-budABC contained 0.4 mM isopropyl β-D-1-thiogalactopyranoside (IPTG). TM3a/glucose medium contains (per liter): 10 g glucose, 13.6 g KH₂PO₄, 2.0 g citric acid monohydrate, 3.0 g (NH₄)₂SO₄, 2.0 g MgSO₄.7H₂O, 0.2 g CaCl₂.2H₂O, 0.33 g ferric ammonium citrate, 1.0 mg thiamine HCl, 0.50 g yeast extract, and 10 mL trace elements solution, adjusted to pH 6.8 with NH₄OH. The solution of trace elements contained: citric acid H₂O (4.0 g/L), MnSO₄.H₂O (3.0 g/L), NaCl (1.0 g/L), FeSO₄.7H₂O (0.10 g/L), COCl₂.6H₂O (0.10 g/L), ZnSO₄.7H₂O (0.10 g/L), CuSO₄.5H₂O (0.010 g/L), H₃BO₃ (0.010 g/L), and Na₂MoO₄ 2H₂O (0.010 g/L). The flasks, capped with vented caps, were inoculated at a starting OD₆₀₀ of approximately 0.03 units and incubated at 34° C. with shaking at 300 rpm.

Approximately 23 h after induction, an aliquot of the broth was analyzed by HPLC (Shodex Sugar SH1011 column) and GC(HP-INNOWax), using the same methods described in the General Methods section for 2-butanol and 2-butanone. The results of the analysis are given in Table 14. The three E. coli clones converted glucose to acetoin and meso-2,3-butanediol, the desired intermediates of the pathway, with a molar selectivity of 14%. This selectivity was approximately 35-fold higher than that observed with the E. coli control strain lacking budABC.

TABLE 14
Production of Acetoin and meso-2,3-butanediol
by E. coli Top 10/pTrc99a-budABC

| | | | Meso-2,3- | |
|---|---|---|---|---|
| | | | Butanediol, | Molar |
| Strain | OD₆₀₀ | Acetoin, mM | mM | Selectivity^{a}, % |
| Negative | 1.4 | 0.07 | 0.03 | 0.4 |
| control | | | | |
| Isolate #1 | 1.5 | 0.64 | 1.3 | 14 |
| Isolate #2 | 1.4 | 0.70 | 1.2 | 14 |
| Isolate #3 | 1.4 | 0.74 | 1.3 | 15 |

^{a}Molar selectivity is (acetoin + meso-2,3-butanendiol)/(glucose consumed).

Construction of Vector pCL1925-KoDD-ddr:

The diol dehydratase (GenBank D45071, SEQ ID NO:69) and reactivating factor (GenBank AF017781, SEQ ID NO:70) operons were PCR amplified from Klebsiella oxytoca ATCC 8724 as a single unit with primers DDo For (SEQ ID NO: 44) and DDo Rev (SEQ ID NO:45). The forward primer incorporated an optimized E. coli RBS and a HindIII restriction site. The reverse primer included an XbaI restriction site. The 5318 bp PCR product was cloned into pCR4 Blunt-TOPO and clones of the resulting pCR4 Blunt-TOPO-Kodd-ddr were sequenced with primers M13 Forward (SEQ ID NO:35), M13 Reverse (SEQ ID NO:36), DDko seq F2 (SEQ ID NO:46), DDko seq F5 (SEQ ID NO:47), DDko seq F7 (SEQ ID NO:48), DDko seq F9 (SEQ ID NO:49), DDko seq R1 (SEQ ID NO:50), DDko seq R3 (SEQ ID NO:51), DDko seq R7 (SEQ ID NO:52), and DDko seq R10 (SEQ ID NO:53). A clone having the insert with the expected sequence was identified.

For expression, the diol dehydratase/reactivating factor genes were subcloned into pCL1925 (U.S. Pat. No. 7,074,608), a low copy plasmid carrying the glucose isomerase promoter from Streptomcyes. pCR4 Blunt-TOPO-Kodd-ddr was digested with HindIII and XbaI and the resulting 5.3 kbp Kodd-ddr fragment was gel-purified. Vector pCL1925 was digested with HindIII and XbaI and the resulting 4539 bp vector fragment was gel purified. The vector and Kodd-ddr fragment were ligated and transformed into E. coli Top10. Transformants were screened by PCR with primers DDko Seq F7 (SEQ ID NO:48) and DDko Seq R7 (SEQ ID NO: 52). Amplification of the plasmid (pCL1925-Kodd-ddr) carrying the insert resulted in a product of approximately 797 bp.

Activity of diol dehydratase towards meso-2,3-butanediol was measured by incubating cell extract (total protein ˜0.8 mg/mL) with 10 mM butanediol and 12 mM coenzyme B₁₂ in 80 mM HEPES (pH 8.2) for 17 h at room temperature. Formation of the expected product, 2-butanone, was determined by HPLC as described in the General Methods.

Construction of vector pCL1925-KoDD-ddr::T5 chnA ter:

To provide a heterologous alcohol dehydrogenase activity, the chnA gene encoding cyclohexanol dehydrogenase from Acinetobacter sp. (Cheng et al., J. Bacteriol. 182:4744-4751 (2000)) was cloned into the pCL1925 vector with the diol dehydratase operon, pCL1925-Kodd-ddr. The chnA gene, given as SEQ ID NO:71 (Genbank No: AF282240, SEQ ID NO:73) was amplified from pDCQ2, a cosmid carrying the cyclohexanol gene cluster from Acinetobacter, with primers ChnA F (SEQ ID NO:54) and ChnA R (SEQ ID NO:55). The resulting 828 bp PCR product was cloned into pCR4 Blunt-TOPO to create pCR4 Blunt-TOPO-chnA and transformants were screened by colony PCR with primers M13 Forward (SEQ ID NO:35) and M13 Reverse (SEQ ID NO:36). Correct clones produced a PCR product of about 1 kbp and were sequenced with primers M13 Forward (SEQ ID NO:35) and M13 Reverse (SEQ ID NO:36).

After sequencing pCR4 Blunt-TOPO-chnA to confirm the correct sequence, the chnA gene was subcloned from the plasmid as an 813 bp Mfel/SmaI fragment. The expression vector pQE30 (Qiagen) was digested with Mfel and SmaI and the resulting 3350 bp vector fragment was gel-purified. The chnA fragment and the purified vector were ligated and transformed into E. coli Top10 cells. Transformants were colony PCR screened with primers chnSeq F1 (SEQ ID NO:56) and chnseq R1 (SEQ ID NO:57) for a 494 bp PCR product. This cloning placed the chnA gene under the control of the T5 promoter in the plasmid, pQE30-chnA.

To prepare the pCL1925 vector to carry two operons, terminators were added to the vector. A tonB terminator-mcs-trpA terminator fragment was prepared by oligonucleotide annealing with primers Top ter F1 (SEQ ID NO:58), Top ter F2 (SEQ ID NO:59), Bot ter R1 (SEQ ID NO:60) and Bot ter R2 (SEQ ID NO:61). The annealed DNA was gel-purified on a 6% PAGE gel (Embi-tec, San Diego, Calif.). Vector pCL1925 was digested with SacI and XbaI and gel-purified. The annealed DNA and vector fragment were ligated to create pCL1925-ter. Transformants were screened by colony PCR amplification with primers pCL1925 vec F (SEQ ID NO:62) and pCL1925 vec R1 (SEQ ID NO:63) for the presence of a PCR product of approximately 400 bp. Positive clones from the PCR screen were sequenced with the same primers.

Vector pCL1925-ter was digested with XhoI and PmeI and the resulting 4622 bp fragment was gel-purified. pQE30-chnA was digested with NcoI and the DNA was treated with Klenow DNA polymerase to blunt the ends. pQE30-chnA was then digested with XhoI and the resulting 1.2 kbp T5 promoter-chnA fragment was gel-purified. The pCL1925-ter vector and the chnA operon fragment were ligated together to give pCL1925-ter-T5chnA and transformed into E. coli Top10. Transformants were screened by colony PCR amplification with primers pCL1925 vec F (SEQ ID NO:64) and chnseq R1 (SEQ ID NO:59) for a product of approximately 1 kbp.

To finish building the pathway vector, the pCL1925-KoDD-ddr plasmid was digested with XbaI and SacI and the resulting 9504 bp vector fragment was gel-purified. The chnA operon flanked by terminators, with the trpA terminator (Koichi et al. (1997) Volume 272, Number 51, pp. 32034-32041) 3′ to the chnA coding sequence, from pCL1925-ter-T5chnA was gel-purified as a 1271 bp XbaI/SacI fragment. After ligation of the fragments and transformation into E. coli Top10, transformants were screened by colony PCR. Primers chnseq F1 (SEQ ID NO:58) and pCL1925 vec R2 (SEQ ID NO:64) amplified the expected 1107 bp PCR product in the resulting plasmid, pCL1925-KoDD-ddr::ter-T5chnA.

### Example 14

### Expression of a 2-Butanol Biosynthetic Pathway in E. coli with Overexpressed Endogenous Alcohol Dehydrogenase

The purpose of this Example was to express a 2-butanol biosynthetic pathway in several E. coli strains.

Construction of E. coli Strains Constitutively Expressing yqhD:

E. coli contains a native gene (yqhD) that was identified as a 1,3-propanediol dehydrogenase (U.S. Pat. No. 6,514,733). The yqhD gene, given as SEQ ID NO:74, has 40% identity to the gene adhB in Clostridium, a probable NADH-dependent butanol dehydrogenase. The yqhD gene was placed under the constitutive expression of a variant of the glucose isomerase promoter 1.6Gl (SEQ ID NO:67) in E. coli strain MG1655 1.6yqhD::Cm (WO 2004/033646) using λ Red technology (Datsenko and Wanner, Proc. Natl. Acad. Sci. U.S.A. 97:6640 (2000)). Similarly, the native promoter was replaced by the 1.5Gl promoter (WO 2003/089621) (SEQ ID NO:68), creating strain MG1655 1.5yqhD::Cm, thus, replacing the 1.6Gl promoter of MG1655 1.6yqhD::Cm with the 1.5Gl promoter. The 1.5Gl and 1.6Gl promoters differ by 1 bp in the −35 region, thereby altering the strength of the promoters (WO 2004/033646). While replacing the native yqhD promoter with either the 1.5Gl or 1.6Gl promoter, the yqhc gene encoding the putative transcriptional regulator for the yqh operon was deleted. Butanol dehydrogenase activity was confirmed by enzyme assay using methods that are well known in the art.

Transformation of E. coli Strains:

Pathway plasmids pCL1925-Kodd-ddr and pTrc99a-budABC, described in Example 13, were co-transformed into E. coli strains MG1655, MG1655 1.6yqhD, and MG1655 1.5yqhD. The two latter strains overexpress the 1,3-propanediol dehydrogenase, YqhD, which also has butanol dehydrogenase activity. Strains were examined for the production of 2-butanone and 2-butanol essentially as described above. Cells were inoculated into shake flasks (approximately 175 mL total volume) containing either 50 or 150 mL of TM3a/glucose medium (with 0.1 mg/L vitamin B₁₂, appropriate antibiotics and IPTG) to represent medium and low oxygen conditions, respectively. Spectinomycin (50 μg/mL) and carbenicillin (100 μg/mL) were used for plasmids pCL1925-Kodd-ddr and pTrc99a-budABC, respectively. The flasks were inoculated at a starting OD₆₀₀ of ≦0.04 units and incubated at 34° C. with shaking at 300 rpm. The flasks containing 50 mL of medium were capped with vented caps; the flasks containing 150 mL, were capped with non-vented caps to minimize air exchange. IPTG was present at time zero at a concentration of zero or 0.04 mM. Analytical results for 2-butanone and 2-butanol production are presented in Table 15. All the E. coli strains comprising a 2-butanol biosynthetic pathway produced 2-butanone under low and medium oxygen conditions and produced 2-butanol under low oxygen conditions.

TABLE 15
Production of 2-Butanone and 2-Butanol by E. coli MG1655 strains
harboring pathway plasmids pCL1925-Kodd-ddr and pTrc99a-budABC

| | | Volume of | 2-Butanone, | 2-Butanol, |
|---|---|---|---|---|
| Strain^{a,b} | IPTG, mM | Medium, mL | mM | mM |
| MG1655 #1 | 0 | 50 | 0.08 | Not detected |
| MG1655 #2 | 0 | 50 | 0.11 | Not detected |
| MG1655 #1 | 0.04 | 50 | 0.12 | Not detected |
| MG1655 #2 | 0.04 | 50 | 0.11 | Not detected |
| MG1655 #1 | 0 | 150 | 0.15 | 0.047 |
| MG1655 #2 | 0 | 150 | 0.19 | 0.041 |
| MG1655 #1 | 0.04 | 150 | 0.10 | 0.015 |
| MG1655 #2 | 0.04 | 150 | 0.11 | 0.015 |
| MG1655 | 0 | 50 | 0.10 | Not detected |
| 1.5yqhD #1 | | | | |
| MG1655 | 0 | 50 | 0.07 | Not detected |
| 1.5yqhD #2 | | | | |
| MG1655 | 0.04 | 50 | 0.12 | Not detected |
| 1.5yqhD #1 | | | | |
| MG1655 | 0.04 | 50 | 0.18 | Not detected |
| 1.5yqhD #2 | | | | |
| MG1655 | 0 | 150 | 0.16 | 0.030 |
| 1.5yqhD #1 | | | | |
| MG1655 | 0 | 150 | 0.18 | 0.038 |
| 1.5yqhD #2 | | | | |
| MG1655 | 0.04 | 150 | 0.10 | 0.021 |
| 1.5yqhD #1 | | | | |
| MG1655 | 0.04 | 150 | 0.09 | 0.017 |
| 1.5yqhD #2 | | | | |
| MG1655 | 0 | 50 | 0.08 | Not detected |
| 1.6yqhD #1 | | | | |
| MG1655 | 0 | 50 | 0.07 | Not detected |
| 1.6yqhD #2 | | | | |
| MG1655 | 0.04 | 50 | 0.12 | Not detected |
| 1.6yqhD #1 | | | | |
| MG1655 | 0.04 | 50 | 0.15 | Not detected |
| 1.6yqhD #2 | | | | |
| MG1655 | 0 | 150 | 0.17 | 0.019 |
| 1.6yqhD #1 | | | | |
| MG1655 | 0 | 150 | 0.18 | 0.041 |
| 1.6yqhD #2 | | | | |
| MG1655 | 0.04 | 150 | 0.11 | 0.026 |
| 1.6yqhD #1 | | | | |
| MG1655 | 0.04 | 150 | 0.11 | 0.038 |
| 1.6yqhD #2 | | | | |
| Control | | | Not detected | Not detected |
| (uninoculated | | | | |
| medium) | | | | |

^{a}#1 and #2 represent independent isolates.
^{b}MG1655 is MG1655/pCL1925-Kodd-ddr/pTrc99a-budABC
MG1655 1.6yqhD is MG1655 1.6yqhD/pCL1925-Kodd-ddr/pTrc99a-budABC
MG1655 1.6yqhD is MG1655 1.5yqhD/pCL1925-Kodd-ddr/pTrc99a-budABC.

### Example 15

### Expression of a 2-Butanol Biosynthetic Pathway in E. coli with Heterologous Alcohol Dehydrogenase

Plasmids pCL1925-KoDD-ddr::ter-T5chnA and pTrc99a-budABC, described in Example 13, were transformed into E. coli strains MG1655 and MG1655 ΔyqhCD for a demonstration of the production of 2-butanol.

MG1655 ΔyqhCD carries a yqhCD inactivation that was made using the method of Datsenko and Wanner (Proc. Natl. Acad. Sci. U.S.A. 97(12):6640-6645 (2000)). After replacement of the region with the FRT-CmR-FRT cassette of pKD3, the chloramphenicol resistance marker was removed using the FLP recombinase. The sequence of the deleted region is given as SEQ ID NO:66.

Strains MG1655/pTrc99a-budABC/pCL1925KoDD-ddr::ter-T5 chnA and MG1655 ΔyqhCD/pTrc99a-budABC/pCL1925KoDD-ddr::ter-T5 chnA were examined for the production of 2-butanone and 2-butanol essentially as described above. Strain MG1655 ΔyqhCD/pCL1925 was used as a negative control. Cells were inoculated into shake flasks (approximately 175 mL total volume) containing 50 or 150 mL of TM3a/glucose medium (with 0.1 mg/L vitamin B₁₂ and appropriate antibiotics) to represent medium and low oxygen conditions, respectively. Spectinomycin (50 μg/mL) and ampicillin (100 μg/mL) were used for selection of pCL1925 based plasmids and pTrc99a-budABC, respectively. Enzyme activity derived from pTrc99a-budABC was detected by enzyme assay in the absence of IPTG inducer, thus, IPTG was not added to the medium. The flasks were inoculated at a starting OD₆₀₀ of ≦0.01 units and incubated at 34° C. with shaking at 300 rpm for 24 h. The flasks containing 50 mL of medium were capped with vented caps; the flasks containing 150 mL, were capped with non-vented caps to minimize air exchange. Analytical results for 2-butanone and 2-butanol production are presented in Table 16. Both E. coli strains comprising a 2-butanol biosynthetic pathway produced 2-butanone under low and medium oxygen conditions and produced 2-butanol under low oxygen conditions, while the negative control strain did not produce detectable levels of either 2-butanone or 2-butanol.

TABLE 16
Production of 2-butanone and 2-butanol by E. coli strains

| | | 2- | |
|---|---|---|---|
| | Volume, | Butanone, | 2-Butanol, |
| Strain^{a} | mL | mM | mM |
| Negative control, MG1655 | 50 | Not | Not detected |
| ΔyqhCD/pCL1925 | | detected | |
| MG1655/pTrc99a- | 50 | 0.33 | Not detected |
| budABC/pCL1925KoDD-ddr::T5 | | | |
| chnA ter | | | |
| MG1655 ΔyqhCD/pTrc99a- | 50 | 0.23 | Not detected |
| budABC/pCL1925KoDD-ddr::T5 | | | |
| chnA ter #1 | | | |
| MG1655 ΔyqhCD/pTrc99a- | 50 | 0.19 | Not detected |
| budABC/pCL1925KoDD-ddr::T5 | | | |
| chnA #2 | | | |
| Negative control, MG1655 | 150 | Not | Not detected |
| ΔyqhCD/pCL1925 | | detected | |
| MG1655/pTrc99a- | 150 | 0.41 | 0.12 |
| budABC/pCL1925KoDD-ddr::T5 | | | |
| chnA ter | | | |
| MG1655 ΔyqhCD/pTrc99a- | 150 | 0.15 | 0.46 |
| budABC/pCL1925KoDD-ddr::T5 | | | |
| chnA #1 | | | |
| MG1655 ΔyqhCD/pTrc99a- | 150 | 0.44 | 0.14 |
| budABC/pCL1925KoDD-ddr::T5 | | | |
| chnA #2 | | | |
| Medium | | Not | Not detected |
| | | detected | |

^{a}#1 and #2 represent independent isolates.

### Example 16

### Cloning of Amino:Pyruvate Transaminase (APT)

An amino:pyruvate transaminase (APT) from Vibrio Fluvialis JS17 was identified by Shin et al. (Appl. Microbiol. Biotechnol. (2003) 61:463-471). The amino acid sequence (SEQ ID NO:122) was found to have significant homology with ω-amino acid:pyruvate transaminases (Shin and Kim (J. Org. Chem. 67:2848-2853 (2002)). It was shown that the Vibrio Fluvialis APT has transaminase activity towards acetoin.

For expression of the APT enzyme in E. coli, a codon optimized APT coding region (SEQ ID NO:144) was designed using the preferred E. coli codons with additional considerations such as codon balance and mRNA stability, and synthesized (by DNA2.0; Redwood City, Calif.). The coding region DNA fragment was subcloned into the pBAD.HisB vector (Invitrogen) between the NcoI and HindIII sites and the resulting plasmid, hereafter referred to as pBAD.APT1, was transformed into TOP10 cells.

### Example 17

### Characterization of Vibrio Fluvialis APT Alanine:Acetoin Aminotransferase Activity

A 5 mL volume of LB broth+100 μg/mL ampicillin was inoculated with a fresh colony of TOP10/pBAD:APT1 cells. The culture was incubated at 37° C. for approximately 16 h with shaking (225 rpm). A 300 μL aliquot of this culture was used to inoculate 300 mL of the same medium, which was incubated at 37° C. with shaking (225 rpm). When the culture reached an OD₆₀₀ of 0.8, L-arabinose was added to a final concentration of 0.2% (w/v). The culture was incubated for an additional 16 h, then harvested. The cells were washed once with 100 mM potassium phosphate buffer (pH 7.8) and then frozen and stored at −80° C.

To isolate the enzyme, the cell pellet was thawed and resuspended in 8 mL of 100 mM potassium phosphate buffer (pH 7) containing 0.2 mM ethylenediaminetetraacetate, 1 mM dithiothreitol and 1 tablet of protease inhibitor cocktail (Roche; Indianapolis, Ind.). The cells were lysed by two passes through a French pressure cell at 900 psi, and the resulting lysate was clarified by centrifugation for 30 min at 17000×g. Ammonium sulfate was added to 35% saturation, and the solution was stirred for 30 min at room temperature, at which point precipitated solids were removed by centrifugation (30 min, 17000×g). Additional ammonium sulfate was added to the supernatant to give 55% saturation, and the solution was again stirred for 30 min at room temperature. The precipitated solids were removed by centrifugation (30 min, 17000×g) and then resuspended in 5 mL of 100 mM potassium phosphate buffer (pH 7) containing 10 μM pyridoxal 5′-phosphate and 1 mM dithiothreitol. This solution was desalted by passage through a PD10 column equilibrated with Buffer A (50 mM bis-tris propane buffer (pH 6) containing 10 μM pyridoxal 5′-phosphate and 1 mM dithiothreitol). The desalted extract was then loaded onto a 20 mL Q-Fast Flow column pre-equilibrated with Buffer A. APT was eluted with a linear gradient of 0-0.1 M NaCl in Buffer A. The enzyme was detected in eluted fractions by the presence of a protein band of size ˜50 kD when analyzed by SDS-polyacrylamide gel electrophoresis and by the characteristic absorbance at 418 nm. Fractions containing the enzyme eluted at ˜0.3 M NaCl. These fractions were pooled to yield a total of 6 mL of a 5.45 mg/mL solution of enzyme, which was >90% pure, as judged by SDS-polyacrylamide gel electrophoresis.

The alanine:acetoin aminotransferase activity of APT was assayed using a lactic dehydrogenase coupled assay. Reaction mixtures contained 100 mM bis-tris propane (pH 9.0), 10 μM pyridoxal 5′-phosphate, 0-50 mM acetoin, 0-5 mM L-alanine, 0.14 or 0.28 mg/mL purified enzyme, 200 μM NADH and 20 U/mL lactic dehydrogenase (Sigma; St. Louis, Mo.). The reaction was followed by measuring the change in absorbance at 340 nm, indicative of the oxidation of NADH. Under these conditions, the k_{cat}/Kₘ for acetoin was 10 M⁻¹ s⁻¹ and that for L-alanine was 400 M⁻¹ s⁻¹.

The identity of the expected product 3-amino-2-butanol was confirmed by comparison to a synthetic standard. A mixture of (R,R)- and (S,S)-3-amino-2-butanol was synthesized by the method of Dickey et al. [J Amer Chem Soc 74:944 (1952)]: 5 g of trans-2,3-epoxybutane were slowly stirred into 150 mL of cold (4° C.) NH₄OH. The reaction was slowly warmed to room temperature, sealed and stirred at room temperature for an additional 10 days. At this time, excess ammonia and water and residual epoxybutane were removed by rotary evaporation under vacuum at 40° C. The resulting clear oil (2.9 g) was resuspended in water to a concentration of 10% (w/v). Production of the desired product was confirmed by NMR analysis and comparison of the spectrum to that reported by Levy et al. [Org. Magnetic Resonance 14:214 (1980)]. A mixture of the corresponding (2R,3S)- and (2S,3R)-isomers was produced using the identical method with the exception that the starting material was the cis-isomer of 2,3-epoxybutane.

An analytical method for detection of 3-amino-2-butanol was developed based on the o-phthaldialdehyde derivatization method for amino acid determination reported by Roth [Anal. Chem. 43:880 (1971)]. A 200 μL aliquot of 1 mM 3-amino-2-butanol (mixture of isomers) was mixed with 200 μL of a 50 mM solution of borate (pH 9.5), to which was added 10 μL of 5 μL/mL 2-mercaptoethanol in ethanol and 10 μL of 10 mg/mL o-phthaldialdehdye in ethanol. The solution was incubated at room temperature for 10 min, at which time the derivative was extracted into 200 μL hexane. The hexane was separated from the aqueous solution by decanting, and 10 μL were injected onto a Chiracel OD HPLC column (Daicel Chemical Industries; Fort Lee, N.J.). The column was run isocratically with a mobile phase of 90:10 hexane:isopropanol at a rate of 1 mL/min. The derivatized isomers of 3-amino-2-butanol were detected by absorbance at 340 nm with retention times of approximately 15.7 and 16.8 min [(2S,3S) and (2R,3R)], and 18.4 and 21.9 min [(2R,3S) and (2S,3R)]. To differentiate the enantiomers in the first mixture, the pure (2R,3R) isomer (Bridge Organics; Vicksburg, Mich.) was also run under the identical conditions and found to be the 16.8 min peak. To differentiate the enantiomers in the second mixture, the mixture was first kinetically resolved using the alanine:acetoin aminotransferase: 0.28 mg of purified enzyme was incubated with 10 mM pyruvate and 10 mM 3-amino-2-butanol [1:1 mixture of (2R,3S) and (2S,3R) isomers] in 1 mL of 100 mM bis-tris propane (pH 9.0). After 24 h at room temperature, an aliquot was removed and analyzed as described above. Analysis revealed that the 18.4 min peak was 95% depleted, while the 21.9 min peak was >90% retained. A 100 μL aliquot of the remaining reaction mixture was mixed with 50 μL of 20 mM NADH and 10 μL of extract from the TOP10/pTrc99a-BudC strain described in Example 13. The BudC enzyme is known to reduce (R)-acetoin to meso-2,3-butanediol and (S)-acetoin to (S,S)-2,3-butanediol [Ui et al., (2004) Letters in Applied Microbiology 39:533-537]. After 3 h, samples were taken from the reaction and analyzed as described above for acetoin and butanediol. The analysis indicated that the primary product of the reduction was meso-2,3-butanediol, indicating that the product of the aminotransferase reaction was (R)-acetoin, and therefore the consumed 3-amino-2-butanol isomer was the (2R,3S) isomer. Thus the retention time of 18.4 min can be assigned to this isomer and 21.9 to the (2S,3R) isomer.

To confirm that the product of the APT-catalyzed alanine:acetoin aminotransferase reaction was 3-amino-2-butanol, 0.28 mg of purified enzyme was incubated with 10 mM acetoin, 10 mM L-alanine, 50 U lactic dehydrogenase and 200 μM NADH in 1 mL of 100 mM bis-tris propane (pH 9.0). The reaction mixture was incubated at room temperature for 20 h, after which a 200 μL aliquot was removed and derivatized as described above. The retention times of the derivatized products were 15.8 min (major product) and 18.5 min (minor product), matching that of the (2S,3S)- and (2R,3S)-3-amino-2-butanol standards.

### Example 18

### Identification and Cloning of Erwinia carotovora subsp. atroseptica Amino Alcohol Kinase and Amino Alcohol O-Phosphate Lyase

The purpose of this example is to describe the identification and cloning of sequences encoding an amino alcohol kinase and amino alcohol O-phosphate lyase from the bacterium Erwinia carotovora. These two enzymes are part of Pathway 1 for the conversion of 3-amino-2-butanol to 2-butanone via the intermediate 3-amino-2-butanol phosphate as shown in FIG. 1.

### Prediction of the Erwinia Amino Alcohol Kinase and the Amino Alcohol O-Phosphate Lyase

ATP-dependent amino alcohol kinase and amino alcohol O-phosphate lyase activities have been detected in several Pseudomonas and Erwinia species, including Pseudomonas sp. P6 (NCIB10431), Pseudomonas putida NCIB 10558 (Jones et al. (1973) Biochem. J. 134:167-182), Erwinia carotovora, Erwinia amanas, Erwina milletiae, and Erwinia atroseptica (Jones et al. (1973) Biochem. J. 134:959-968). In these studies, the extracts of the above species were shown to have activity for the enzymatic conversion of aminopropanol through aminopropanol O-phosphate to propionaldehyde, and the conversion of ethanolamine through ethanolamine O-phosphate to acetaldehyde.

The genomic sequence of the Erwinia atroseptica strain in which these activities were reported to exist (now designated as Erwinia carotovora subsp. atroseptica strain SCR11043 (ATCC BAA-672)) has been determined at the Sanger Institute (Bell et al. Proc. Natl. Acad. Sci. USA 101(30): 11105-11110). Analysis of the putative kinases in the Erwinia carotovora subsp. atroseptica genome revealed an operon sequence (SEQ ID NO:154) encoding a putative protein (ECA2059; SEQ ID NO:124) that is 39% identical to a Rhizobium loti homoserine kinase and a putative class-III pyridoxal phosphate (PLP)-dependent aminotransferase (ECA2060; SEQ ID NO:126) that is 58% identical to a putative aminotransferase from Rhizobium meliloti. We predicted that ECA2059 was an amino alcohol kinase and ECA2060 was an amino alcohol O-phosphate lyase which uses PLP as cofactor.

Cloning of the Putative Amino Alcohol Kinase and Putative Amino Alcohol O-Phosphase Lyase from Erwinia carotovora subsp. atroseptica

Genomic DNA of Erwinia carotovora subsp. atroseptica (ATCC #: BAA-672D) was obtained from American Type Culture Collection (ATCC). The operon encoding the putative amino alcohol kinase (KA) and amino alcohol O-phosphate lyase (AT) was named KA-AT (SEQ ID NO:154. This operon was amplified from the Erwinia genomic DNA by Phusion DNA polymerase (Finnzymes; via New England Biolabs; Ipswich, Mass.) using primers OT872 (SEQ. ID. No. 127) and OT873 (SEQ. ID. No128). A DNA fragment of 2.4 kb was obtained by the PCR reaction, which corresponds to the size of the KA-AT operon. The PCR product was digested with EcoRI and PstI restriction enzymes, and cloned into vector pKK223-3 (Amersham Biosciences; Piscataway, N.J.) which was digested with the same restriction enzymes. This produced plasmid pKK223.KA-AT, which contained the putative Erwinia amino alcohol kinase-lyase operon under control of the tac promoter. Similarly, plasmids pKK223.KA and pKK223.AT were made which placed the putative Erwinia kinase and the putative Erwinia lyase coding regions in separate vectors, each under the control of the tac promoter. For the PCR cloning of the KA coding region (SEQ ID NO:123), primers OT872 (SEQ. ID. No. 127) and OT879 (SEQ. ID. No. 129) were used; and for the PCR cloning of AT coding region (SEQ ID NO:125), primers OT873 (SEQ. ID. No. 128) and OT880 (SEQ. ID. No. 130) were used in the PCR amplifications, which generated PCR products of 1.1 kb and 1.3 kb respectively. The PCR products were each digested with EcoRI and PstI, and ligated into vector pKK223-3 to generate pKK223.KA and pKK223.AT.

In Vivo Activity of the Putative Amino Alcohol Kinase and Putative Amino Alcohol O-Phosphate Lyase from Erwinia carotovora Subsp. Atroseptica

Plasmids pKK223.KA-AT, pKK223.KA, pKK223.AT and pKK223-3 were transformed into the E. coli MG1655 strain. The transformants were restreaked onto a MOPS minimal media plate containing 1% glucose, 0.5% aminopropanol as a sole nitrogen source, 1 mM IPTG and 100 μg/mL ampicillin. Expression of KA-AT, KA and AT genes were induced by the IPTG. A control plate had no IPTG included. The plates were incubated at 37° C. for 7 days. On the plate with IPTG, only the strain MG1655/pKK223.KA-AT grew, while all the other three strains did not grow. On the plate without added IPTG, the strain MG1655/pKK223.KA-AT grew, but the colonies were significantly smaller than those on the IPTG-containing plate, which corresponds to the lower expression levels of KA and AT in the uninduced cells. None of the other three strains grew on this plate. This indicates that the co-expression of the putative Erwinia KA and AT genes provided sufficient enzyme activities that allowed the E. coli strain MG1655/pKK223.KA-AT to utilize aminopropanol as a sole nitrogen source. Expression of each individual enzyme of either KA or AT was not sufficient to provide such enzyme activity in vivo.

### Example 19

### In Vitro Activity of Erwinia Putative Amino Alcohol Kinase and Amino Alcohol O-Phosphate Lyase

Subcloning of the Erwinia KA-AT Operon into the pBAD.HisB Vector and Induction of Protein Expression

The protein expression levels of Erwinia putative KA and AT enzymes expressed in MG1655 cells from the pKK223.KA-AT vector were analyzed by SDS-PAGE analysis. The expression level of the Erwinia AT enzyme was relatively low, with a new protein band detected at the correct molecular weight of 46 kD in the soluble fraction of a cell extract, while no new protein band was detected at the size predicted for the KA enzyme.

In an effort to improve the expression of the Erwinia putative KA and AT genes, the KA-AT operon was subcloned into the EcoRI and HindIII sites of vector pBAD.HisB-EcoRI. pBAD.HisB-EcoRI was derived from the pBAD.HisB vector (Invitrogen), by replacing the NcoI site in pBAD.HisB with an EcoRI site via QuickChange site-directed mutagenesis (Stratagene, La Jolla, Calif.) using primers OT909 (SEQ ID.# 131) & OT910 (SEQ ID.# 132). In the constructed plasmid pBAD.KA-AT, the KA-AT operon was placed directly under control of the araB promoter (without His-tag).

The pBAD.KA-AT plasmid was transformed into the E. coli TOP10 strain. A 50 mL culture of TOP10/pBAD.KA-AT strain was grown to mid log phase (OD₆₀₀=0.6) in LB, 100 μg/mL ampicillin media at 37° C. with shaking at 250 rpm. The culture was induced by addition of L-arabinose to a final concentration of 0.1% (w/v), and it was further incubated at 37° C. for 5 h before harvesting by centrifugation. The cell pellet was resuspended in ice cold 50 mM Tris-HCl, pH 8.0, and disrupted by sonication on ice with a Fischer Sonic Model 300 Dismembrator (Fischer, Pittsburgh, Pa.) at 50% power, repeating four cycles of 30 seconds sonication with 60 seconds rest in-between each cycle. Each sonicated sample was centrifuged (15,000×g, 4 min, 4° C.). Clarified cell free extracts were analyzed for protein expression level and amino alcohol O-phosphate lyase activity.

### Chemical Synthesis of Aminobutanol O-Phosphate and Aminopropanol O-Phosphate

The substrate (R,R)-3-amino-2-butanol O-phosphate was synthesized by a method based on that reported by Ferrari and Ferrari (U.S. Pat. No. 2,730,542 [1956]) for phosphoethanolamine: 10 mmol of H₃PO₄ in a 50% (w/v) aqueous solution was mixed with a 50% (w/v) solution of 3-amino-2-butanol (˜20:1 (R,R):(S,S) isomers; Bridge Organics; Vicksburg, Mich.) while stirring on ice. After mixing, the solution was slowly warmed to room temperature and then stirred under vacuum and heated to 70° C. After 1 h at 70° C., the temperature was slowly increased to 185° C. and maintained there for an additional 2 h. At that time, the reaction was cooled to room temperature and the vacuum released. The remaining material was dissolved in water, and analysis by NMR indicated that 80% of the starting material was converted to product with 20% remaining unreacted. No additional products were observed.

The additional substrates (2R,3S)-3-amino-2-butanol O-phosphate and (2S,3R)-3-amino-2-butanol O-phosphate were synthesized by the same procedure using a 1:1 mixture of (2R,3S)-3-amino-2-butanol and (2S,3R)-3-amino-2-butanol (synthesized as described in Example 17) as the starting material. DL-1-amino-2-propanol O-phosphate, (S)-2-amino-1-propanol O-phosphate, and (R)-2-amino-1-propanol O-phosphate were synthesized by the same procedure using DL-1-amino-2-propanol, (R)-2-amino-1-propanol, or (S)-2-amino-1-propanol as the starting material.

Analysis of the Aminopropanol O-Phosphate Lyase Activity Encoded by the Putative Erwinia KA-AT operon

The aminopropanol O-phosphate lyase assay was performed as described by Jones et al. (1973, Biochem. J. 134:167-182) and G. Gori et al. (1995, Chromatographia 40:336) The formation of propionaldehyde from aminopropanol O-phosphate was assayed calorimetrically with MBTH, which allows the detection of aldehyde formation. The reaction was performed as follows. In a 1 mL reaction, 100 μg cell free extract of E. coli TOP10/pBAD.KA-AT was added to 10 mM DL-1-amino-2-propanol O-phosphate in 100 mM Tris-HCl, pH 7.8, with 0.1 mM PLP. The reaction was incubated at 37° C. for 10 min and 30 min, with an aliquot of 100 μL reaction mixture removed at each time point and mixed with 100 μL of 6 mg/mL MBTH in 375 mM glycine-HCl, pH 2.7. This mixture was incubated at 100° C. for 3 min, cooled on ice for 15-30 s, and 1 mL of 3.3 mg/mL FeCl₃.6H₂O (in 10 mM HCl) was added, followed by incubation for 30 min at room temperature. The absorbance of the reaction mixture which contains the aldehyde-MBTH adduct, was measured at 670 nm. The results of the assay are listed in Table 17. In the presence of the aminopropanol phosphate substrate, PLP and cell free extract, formation of aldehyde was detected, as indicated by an Abs₆₇₀ that was higher than the control background of up to 0.3. In the absence of either the substrate or the cell free extract, no aldehyde formation was detected. In the absence of added PLP, somewhat less amount aldehyde was detected, presumably due to the presence of PLP in the cell free extract. Cell free extract of the uninduced TOP10/pBAD.KA-AT—culture did not produce any detectable aldehyde in the reaction. These results indicated that the putative Erwinia amino alcohol O-phosphate lyase does catalyze the conversion of aminopropanol O-phosphate to propionaldehyde.

TABLE 17
Aminopropanol O-phosphate lyase assay. Sample 1 was the cell
free extract of a non-induced control of E. coli
TOP10/pBAD.KA-AT. Samples 2-5 contained the cell free
extract of the induced culture E. coli TOP10/pBAD.KA-AT.

| | | | | Enzyme | | |
|---|---|---|---|---|---|---|
| | Induction | Aminopropanol | | extract | | |
| Sample | by 0.1% | O- | | (100 | OD₆₇₀, | OD₆₇₀, |
| Number | arabinose | phosphate | PLP | μg/mL) | 10 min | 30 min |
| 1 | uninduced | (+) | (+) | (+) | 0.262 | 0.255 |
| 2 | induced | (+) | (+) | (+) | 1.229 | 2.264 |
| 3 | induced | (−) | (+) | (+) | 0.303 | 0.223 |
| 4 | induced | (+) | (−) | (+) | 0.855 | 1.454 |
| 5 | induced | (+) | (+) | (−) | 0.156 | 0.065 |

### Analysis of the Activity of the Erwinia Amino Alcohol O-Phosphate Lyase Towards Aminobutanol O-Phosphate Substrate

The activity of the amino alcohol O-phosphate lyase towards the aminobutanol O-phosphate substrates was studied under the same conditions as described above. The reaction was carried out at 37° C. overnight in a 1 mL reaction that contained 100 μg of cell free extract of E. coli TOP10/pBAD.KA-AT, 10 mM aminobutanol O-phosphate (either the mixture of (R,R)+(S,S) or the mixture of (R,S)+(S,R) isomers described in Example 19) in 100 mM Tris-HCl, pH 7.8, with 0.1 mM PLP. An aliquot of 100 μL reaction mixture was removed and the 2-butanone product was detected using the MBTH derivatization method described in the General Methods. The two peaks representing the derivatized 2-butanone isomers were observed. Therefore the Erwinia amino alcohol O-phosphate lyase is an aminobutanol phosphate phospho-lyase in addition to an aminopropanol phosphate phospho-lyase.

### Analysis of the Activity of the Erwinia Amino Alcohol O-Phosphate Lyase Towards Stereoisomers of Aminopropanol O-Phosphate and Aminobutanol O-Phosphate

The activity of the Erwinia amino alcohol O-phosphate lyase towards various stereoisomers of aminopropanol O-phosphate and aminobutanol O-phosphate was studied under the same conditions as described above. In the presence of the Erwinia amino alcohol O-phosphate lyase, both (R) and (S)-2-amino-1-propanol O-phosphate were converted to propanone by the enzyme, but the product yield was much higher with the (S) isomer. The enzyme also produced butanone from both mixtures of 3-amino-2-butanol O-phosphate isomers, with a higher product yield found in the reaction containing the (R,S) and (S,R) substrate isomers. Both propanone and butanone products were derivatized by MBTH, and detected by HPLC as described in General Methods.

### Optimization of the Gene Expression Level for the Erwinia Amino Alcohol Kinase and Amino Alcohol O-Phosphate Lyase

In order to improve the expression levels for the Erwinia amino alcohol kinase and the amino alcohol O-phosphate lyase in E. coli, codon optimized coding regions for both enzymes (named EKA: SEQ ID NO:155 and EAT: SEQ ID NO:156 respectively) were synthesized by DNA2.0 (Redwood City, Calif.). Each coding region was synthesized with 5′ and 3′ tails including restriction sites for cloning: EKA has 5′ BbsI and 3′ EcoRI, HindIII sites; EAT has 5′ EcoRI and 3′ HindIII sites. The EKA and EAT coding regions were provided from DNA2.0 as plasmids pEKA and pEAT, which were in the pJ51 vector of DNA2.0. The EKA optimized coding region was subcloned by ligating a BbsI and HindIII digested fragment of pEKA into the pBAD.HisB vector between the NcoI and HindIII sites, to generate plasmid pBAD.EKA. In the resulting plasmid the coding region is 5′ to the His tag, so a coding region for an N-terminus His₆ tag fused to the Erwinia amino alcohol kinase was constructed by performing a QuickChange site-directed mutagenesis reaction using primers SEQ ID NO:157 and SEQ ID NO:158 to generate vector pBAD.His-EKA.

pBAD.His-EKA was transformed into E. coli strain BL21-Al (F⁻ ompT hsdSB (rB⁻ mB⁻) gal dcm araB::T7RNAP-tetA; Invitrogen) to produce strain BL21-Al/pBAD.HisA-EKA. A 50 mL culture of BL21-Al/pBAD.HisA-EKA was grown to mid-log stage (OD₆₀₀=0.6), induced with 0.1% arabinose, and further incubated at 30° C. overnight. Cell free extracts were prepared by sonication. The His₆-tagged fusion protein of Erwinia amino alcohol kinase was purified using the ProBond™ Purification System (Invitrogen) under non-denaturing purification conditions following the manufacturer's instructions.

The kinase activity of the His₆-tagged Erwinia amino alcohol kinase is analyzed by the ADP Quest Assay (DiscoveRx, Fremont, Calif.) following the manufacturer's instructions. This is a biochemical assay that measures the accumulation of ADP, a product of the amino alcohol kinase reaction using either aminopropanol or aminobutanol as substrate. 10 mM substrate is mixed with His₆-tagged Erwinia amino alcohol kinase, in 100 mM Tris-HCl, pH 7.8, 10 mM MgCl₂, 2 mM KCl, 0.1 mM ATP, and incubated at 37° C. for 1 h in a 0.2 mL reaction. ADP reagent A (100 μL) and ADP reagent B (200 μL) are added and the mixture is incubated at room temperature for 30 min. The fluorescence signal indicating activity is measured with excitation wavelength of 530 nm and emission wavelength of 590 nm.

### Example 20

### Expression of Entire Pathway 3

### Construction of Vector pCLBudAB-ter-T5chnA

The vector pTrc99a::BudABC (described in Example 13) is digested with EcoRI, and the DNA is treated with Klenow DNA polymerase to blunt the ends. The blunted vector is subsequently digested with SpeI to yield a 2.5 kb fragment containing the budA and budB genes. The vector pCL1925-ter-T5chnA (described in Example 13) is digested with HindIII, and the DNA was treated with Klenow DNA polymerase to blunt the ends. The blunted vector is subsequently digested with XbaI to yield a 4.6 kb fragment which is then ligated to the budAB fragment from pTrc99a::BudABC. The resulting plasmid, designated pCLBudAB-ter-T5chnA, is used to transform E. coli Top10 cells, and single colonies are screened for proper plasmid structure by PCR using primers pCL1925vecF (SEQ ID NO:62) and N84seqR3 (SEQ ID NO:159). Plasmid is prepared from a single colony which yields a PCR product of the expected size of 1.4 kb.

Construction of Vector pKK223.KA-AT-APT

The APT gene is amplified from the vector pBAD.APT (described in Example 16) by PCR using primers APTfor (SEQ ID NO:162; 5′ includes RBS and SmaI site) and APTrev (SEQ ID NO:163; 3′ adds SmaI site). The product of expected size of 1.7 kbp is gel purified and digested with SmaI to yield blunt ends. The vector pKK223.KA-AT (described in Example 18) is digested with PstI, and the DNA is treated with Klenow DNA polymerase to blunt the ends. The resulting DNA fragment is ligated with the SmaI-digested PCR product, and the ligation product is used to transform E. coli Top10 cells. Individual ampicillin resistant colonies are screened by PCR using primers OT872 (SEQ ID NO:127) and APTrev (SEQ ID NO:163). The presence of a PCR product of the expected size of 4.1 kbp indicates that the gene encoding APT is present and oriented in the same direction as the genes encoding KA and AT. The sequence of the insert is verified using the primers APTseqRev (SEQ ID NO:160) and APTseqFor (SEQ ID NO:161). This plasmid is named pKK223.KA-AT-APT. Proper expression of all three genes is verified by growing a 5 mL culture of Top10/pKK223.KA-AT-APT in LB+100 μg/mL ampicillin at 37° C. with shaking. When the OD₆₀₀ reaches ˜0.8, expression of the genes on the plasmid is induced by addition of IPTG to 0.4 mM. The expression is evaluated by SDS PAGE and activity assays as described above.

### Construction of 2-Butanol Production Strain and Production of 2-Butanone and 2-Butanol

E. coli strain MG1655 is transformed with both pKK223.KA-AT-APT and pCLBudAB-ter-T5chnA, and transformants selected for ampicillin and spectinomycin resistance, indicative of the presence of the plasmids. The cells are inoculated into shake flasks (approximately 175 mL total volume) containing 50 or 150 mL of TM3a/glucose medium (with appropriate antibiotics) to represent medium and low oxygen conditions, respectively. IPTG is added to 0.4 mM to induce expression of genes from pKK223.KA-AT-APT. As a negative control, MG1655 cells are grown in the same medium lacking antibiotics. The flasks are inoculated at a starting OD₆₀₀ of ≦0.01 and incubated at 34° C. with shaking at 300 rpm for 24 h. The flasks containing 50 mL of medium are capped with vented caps; the flasks containing 150 mL are capped with non-vented caps to minimize air exchange. The MG1655/pKK223.KA-AT-APT/pCLBudAB-ter-T5chnA strain comprising a 2-butanol biosynthetic pathway produces both 2-butanone and 2-butanol under low and medium oxygen conditions while the negative control strain does not produce detectable levels of either 2-butanone or 2-butanol.

### Example 21

### Characterization of Glycerol Dehydratase Butanediol Dehydratase Activity

Glycerol dehydratase (E.C. 4.2.1.30) and diol dehydratase (E.C. 4.2.1.28), while structurally related, are often distinguished in the art based on various differences that include substrate specificity. This example demonstrates that glycerol dehydratase converts meso-2,3-butanediol to 2-butanone. The recombinant E. coli strain KLP23/pSYCO12, comprising Klebsiella pneumoniae genes encoding the multiple subunits of glycerol dehydratase (alpha: SEQ ID NO:145 (coding region) and 146 (protein); beta: SEQ ID NO: 147 (coding region) and 148 (protein); and gamma: SEQ ID NO: 149 (coding region) and 150 (protein)) and Klebsiella pneumoniae genes encoding the multiple subunits of glycerol dehydratase reactivase (large subunit, SEQ ID NO: 151 (coding region) and 152 (protein); and small subunit, SEQ ID NO: 153 (coding region) and 154 (protein)), is described in Emptage et al. U.S. Pat. No. 6,514,733 and in WO 2003089621, which are herein incorporated by reference. A crude, cell free extract of KLP23/pSYCO12 was prepared by methods known to one skilled in the art. Enzyme assay was performed in the absence of light in 80 mM HEPES buffer, pH 8.2 at 37° C. with 12 μM coenzyme B₁₂ and 10 mM meso-2,3-butanediol. The formation of 2-butanone was monitored by HPLC (Shodex SH-1011 column and SH-G guard column with refractive index detection; 0.01 M H₂SO₄ as the mobile phase at a flow rate of 0.5 mL/min and a column temperature of 50° C.; 2-butanone retention time=40.2 min). The rate of 2-butanone formation by the glycerol dehydratase preparation was determined to be 0.4 nmol/min/mg of crude protein.

What is claimed is:

1. A method for the production of 2-butanone comprising:
a) providing a recombinant microbial production host which produces 2-butanone;
b) seeding the production host of (a) into a fermentation medium comprising a fermentable carbon substrate to create a fermentation culture;
c) growing the production host in the fermentation culture at a first temperature for a first period of time;
d) lowering the temperature of the fermentation culture to a second temperature; and
e) incubating the production host at the second temperature of step (d) for a second period of time;
whereby 2-butanone is produced.

2. A method according to claim 1 wherein the fermentable carbon substrate is derived from a grain or sugar source selected from the group consisting of wheat, corn, barley, oats, rye, sugar cane, sugar beets, cassava, sweet sorghum, and mixtures thereof.

3. A method according to claim 1 wherein the fermentable carbon substrate is derived from cellulosic or lignocellulosic biomass selected from the group consisting of corn cobs, crop residues, corn husks, corn stover, grasses, wheat straw, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, animal manure, and mixtures thereof.

4. A method according to claim 1 wherein the fermentable carbon substrate is selected from the group consisting of monosaccharides, oligosaccharides, and polysaccharides.

5. A method according to claim 1 wherein the fermentation culture is maintained under conditions selected from the group consisting of anaerobic conditions and microaerobic conditions.

6. A method according to claim 1 wherein while growing the production host in (c) at a first temperature over a first period of time, a metabolic parameter of the fermentation culture is monitored.

7. A method according to claim 6 wherein the metabolic parameter that is monitored is selected from the group consisting of optical density, pH, respiratory quotient, fermentable carbon substrate utilization, CO₂ production, and 2-butanone production.

8. A method according to claim 1 wherein lowering the temperature of the fermentation culture of step (d) occurs at a predetermined time.

9. A method according to claim 1 wherein the lowering of the temperature of the fermentation culture of step (d) coincides with a change in a metabolic parameter.

10. A method according to claim 9 wherein the change in metabolic parameter is a decrease in the rate of 2-butanone production.

11. A method according to claim 1 wherein the first temperature is from about 25° C. to about 40° C.

12. A method according to claim 1 wherein the second temperature is from about 3° C. to about 25° C. lower than the first temperature.

13. A method according to claim 1 wherein steps (d) and (e) are repeated one or more times.

14. A method according to claim 1 wherein the recombinant microbial production host is selected from the group consisting of Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Saccharomyces, and Pichia.

15. A method according to claim 1 wherein the recombinant microbial host cell comprises at least one DNA molecule encoding a polypeptide that catalyzes a substrate to product conversion selected from the group consisting of:
i) pyruvate to alpha-acetolactate (pathway step a),
ii) alpha-acetolactate to acetoin (pathway step b),
iii) acetoin to 3-amino-2-butanol (pathway step c),
iv) 3-amino-2-butanol to 3-amino-2-butanol phosphate (pathway step d), and
v) 3-amino-2-butanol phosphate to 2-butanone (pathway step e),
wherein the at least one DNA molecule is heterologous to said microbial production host cell.

16. A method according to claim 15 wherein the polypeptide that catalyzes a substrate to product conversion of pyruvate to alpha-acetolactate is acetolactate synthase.

17. A method according to claim 15 wherein the polypeptide that catalyzes a substrate to product conversion of alpha-acetolactate to acetoin is acetolactate decarboxylase.

18. A method according to claim 15 wherein the polypeptide that catalyzes a substrate to product conversion of acetoin to 3-amino-2-butanol is acetoin aminase.

19. A method according to claim 15 wherein the polypeptide that catalyzes a substrate to product conversion of 3-amino-2-butanol to 3-amino-2-butanol phosphate is aminobutanol kinase.

20. A method according to claim 15 wherein the polypeptide that catalyzes a substrate to product conversion of 3-amino-2-butanol phosphate to 2-butanone is aminobutanol phosphate phospho-lyase.

21. A method according to claim 16 wherein the acetolactate synthase has an amino acid sequence having at least 95% identity to an amino acid sequence selected from the group consisting of SEQ ID NO:4, SEQ ID NO:77, and SEQ ID NO:79 based on the Clustal W method of alignment using the default parameters of GAP PENALTY=10, GAP LENGTH PENALTY=0.1, and Gonnet 250 series of protein weight matrix.

22. A method according to claim 17 wherein the acetolactate decarboxylase has an amino acid sequence having at least 95% identity to an amino acid sequence selected from the group consisting of SEQ ID NO:2, SEQ ID NO: 81, and SEQ ID NO:83 based on the Clustal W method of alignment using the default parameters of GAP PENALTY=10, GAP LENGTH PENALTY=0.1, and Gonnet 250 series of protein weight matrix.

23. A method according to claim 18 wherein the acetoin aminase has an amino acid sequence having at least 95% identity to an amino acid sequence as set forth in SEQ ID NO:122 based on the Clustal W method of alignment using the default parameters of GAP PENALTY=10, GAP LENGTH PENALTY=0.1, and Gonnet 250 series of protein weight matrix.

24. A method according to claim 19 wherein the aminobutanol kinase has an amino acid sequence having at least 95% identity to an amino acid sequence as set forth in SEQ ID NO:124 based on the Clustal W method of alignment using the default parameters of GAP PENALTY=10, GAP LENGTH PENALTY=0.1, and Gonnet 250 series of protein weight matrix.

25. A method according to claim 20 wherein the aminobutanol phosphate phospho-lyase has an amino acid sequence having at least 95% identity to an amino acid sequence as set forth in SEQ ID NO:126 based on the Clustal W method of alignment using the default parameters of GAP PENALTY=10, GAP LENGTH PENALTY=0.1, and Gonnet 250 series of protein weight matrix.

26. A method according to claim 1 wherein the recombinant microbial host cell comprises at least one DNA molecule encoding a polypeptide that catalyzes a substrate to product conversion selected from the group consisting of:
i) pyruvate to alpha-acetolactate (pathway step a),
ii) alpha-acetolactate to acetoin (pathway step b),
iii) acetoin to 2,3-butanediol (pathway step i), and
iv) 2,3-butanediol to 2-butanone (pathway step j);
wherein the at least one DNA molecule is heterologous to said microbial production host cell.

27. A method according to claim 26 wherein the polypeptide that catalyzes a substrate to product conversion of pyruvate to alpha-acetolactate is acetolactate synthase.

28. A method according to claim 26 wherein the polypeptide that catalyzes a substrate to product conversion of alpha-acetolactate to acetoin is acetolactate decarboxylase.

29. A method according to claim 26 wherein the polypeptide that catalyzes a substrate to product conversion of acetoin to 2,3-butanediol is butanediol dehydrogenase.

30. A method according to claim 26 wherein the polypeptide that catalyzes a substrate to product conversion of 2,3-butanediol to 2-butanone is diol dehydratase or glycerol dehydratase.

31. A method according to claim 27 wherein the acetolactate synthase has an amino acid sequence having at least 95% identity to an amino acid sequence selected from the group consisting of SEQ ID NO:4, SEQ ID NO:77, and SEQ ID NO:79 based on the Clustal W method of alignment using the default parameters of GAP PENALTY=10, GAP LENGTH PENALTY=0.1, and Gonnet 250 series of protein weight matrix.

32. A method according to claim 28 wherein the acetolactate decarboxylase has an amino acid sequence having at least 95% identity to an amino acid sequence selected from the group consisting of SEQ ID NO:2, SEQ ID NO: 81, and SEQ ID NO:83 based on the Clustal W method of alignment using the default parameters of GAP PENALTY=10, GAP LENGTH PENALTY=0.1, and Gonnet 250 series of protein weight matrix.

33. A method according to claim 29 wherein the butanediol dehydrogenase has an amino acid sequence having at least 95% identity to an amino acid sequence selected from the group consisting of SEQ ID NO:6, SEQ ID NO:85, SEQ ID NO:87, and SEQ ID NO:89 based on the Clustal W method of alignment using the default parameters of GAP PENALTY=10, GAP LENGTH PENALTY=0.1, and Gonnet 250 series of protein weight matrix.

34. A method according to claim 30 wherein the diol dehydratase or glycerol dehydratase comprises fused large, medium and small subunits and has at least 95% identity to an amino acid sequence comprising all three of the amino acid sequences encoding large, medium and small subunits, selected from the group consisting of:
a) SEQ ID NO:8, SEQ ID NO:10, and SEQ ID NO:12;
b) SEQ ID NO:93, SEQ ID NO:95, and SEQ ID NO:97;
c) SEQ ID NO:99, SEQ ID NO:101, and SEQ ID NO:103;
d) SEQ ID NO:105, SEQ ID NO:107, and SEQ ID NO:109;
e) SEQ ID NO:135, SEQ ID NO:136, and SEQ ID NO:137;
f) SEQ ID NO:138, SEQ ID NO:139, and SEQ ID NO:140;
g) SEQ ID NO:146, SEQ ID NO:148, and SEQ ID NO:150;
h) SEQ ID NO:141, SEQ ID NO:142, and SEQ ID NO:143; and
i) SEQ ID NO:164, SEQ ID NO:165, and SEQ ID NO:166;
based on the Clustal W method of alignment using the default parameters of GAP PENALTY=10, GAP LENGTH PENALTY=0.1, and Gonnet 250 series of protein weight matrix.
